SEQUENCE LISTING (130 sequences)

SEQ ID NO: 1 - DNA, 366 bp, Artificial sequence
  Synthetic antibody
caggtgcagctggtgcagtctggggctgaggtgaagaagcctggggccccagtgaaggtctcctgcgaggcctctggatacacattcaccgactattttatacactgggtgcgacaggctcctggacaaggacttgagtggatgggatggatcaaccctatcagtggtggcacaaactatcacccgagatttcatggcggggtcaccatgaccagggacacctccatgaaagtagcctacatggaacttaagaggctgacatctgacgacacggccgtgtatttctgtgcgagaggtcgagattttaggggtggttattcccaacttgactattggggccagggaaccctggtcaccgtctcctca

SEQ ID NO: 2 - DNA, 330 bp, Artificial sequence
  Synthetic antibody
cagtctgtgctgactcagccaccctcagcgtctgggacccccgggcagagggtcaccatctcttgttctggaggcagctccaacatcgcaattaatactgtaaactggtatcagcaggtcccaggaacggcccccaaactcctcatgtatagtaataatcagcggccctcaggggtccccgaccgattctctggctccaagtctggcacctcagcctccctggccatcagtgggctccagtctgaggatgaggctgattattactgtgcaacatgggatgacagtctgaaagatgtgctattcggcggagggaccaaactgaccgtccta

SEQ ID NO: 3 - DNA, 363 bp, Artificial sequence
  Synthetic antibody
caggtgcagctgcaggagtcgggcccaggactggtgaagccttcggagaccctgtccctcacctgcactgtctctggtgcctccatcagttcttactcgtggagctggatccggcagcccgccgggaggggacttgagtggcttgggcgtatctatcccagtgggaacaccaactacagtccctccctcaagagtcgactcaccatgtcactagacacatccaagaaccagttctccatgaagctgacctctgtgagcgccgcggacacggccgtctattactgtgcgagagatcgggagcagtggcccttgtattatggtatggacgtctggggccaagggaccctggtcaccgtctcctcc

SEQ ID NO: 4 - DNA, 321 bp, Artificial sequence
  Synthetic antibody
gaaattgtgttgacacagtctccagccatcctgtctttgtctccaggggacagagccaccctctcctgcagggccagtcagagtgttttcacctacttagcctggtaccaacataaacctggccaggctcccaggctcctcatctatgatgcatccaacagggcctctggcatcccagccaggttcagtggcagtgggtctgggacagacttcactctcaccatcagcagcctagagcctgaagattttgcagtttattactgtcagcagcgtaccaagtggcccctggctttcggcggagggaccaaggtggagatcaag

SEQ ID NO: 5 - DNA, 366 bp, Artificial sequence
  Synthetic antibody
caggtgcagctgcaggagtcgggcccaggactggtgaagccttcggagaccctgtccctcagttgcactgtctctggtggctccatcagtcctgactactggagctggatccggcagcccccagggaagggactggagtggcttgggtacatctattctgctgggagcaccagctacaacccctccctcaagagtcgagtcaccatgtcagtagacacgtccaagaaccagttatccctgaaactgacctctgtgaccgctgcggacacggccgtgtattactgtgcgaggacggcggggagtttttggagtggtcgaggctggttcgacccctggggccagggaaccctggtcaccgtctcctca

SEQ ID NO: 6 - DNA, 342 bp, Artificial sequence
  Synthetic antibody
gaaattgtgttgacgcagtctccagccaccctgtctttgtctccaggggaaagagtcaccctctcctgcggggccagtcagagtgttagcagcagccacttagcctggtaccagcagaaacctggcctggcgcccaggctcctcatctatgatgcatccaacagggccactggcgtcccagacaggttcagtggcagtgggtctgggacagacttcacactcaccatcagcagactggagcctgaagattttgcagtgtattactgtcaacagtatggtagcccgcagtacacttttggccaggggaccaagctggagatcaaacgaactgtggctgcacca

SEQ ID NO: 7 - DNA, 372 bp, Artificial sequence
  Synthetic antibody
cagtgtcaggtggagctggtggagtctgggggcgacgtggtccagcctgggaagtccctgagactctcctgtgcggcctctggattcaccttcactaactatgctatgcactggctccgccaggctccaggcaaggggctggagtgggtggcagtcatatcttctgatgtcaacgataaatactacgcagactccgtgaagggccgattcaccatctccagagacaattccaagaacaccctgtatctgcaaatgaacagcctgacacctgaagacacggctgtgtattactgtgcgagagagcaagccgtgggaacaaatccgtgggcctttgactactggggccagggaaccctggtcaccgtctcctca

SEQ ID NO: 8 - DNA, 339 bp, Artificial sequence
  Synthetic antibody
cacattgtgatgacccagagtccactctctctgtccgtcacccctggacagccggcctccatctcctgcaagtctagtcagatctcctcttggggtagtgatggaaagacctatttgtattggtacctgcagaagccaggccagtctccacagctcctaatctatgaagtttccagccgattctctggagtgtcagataggttcagtggcagcgggtcagggacagatttcacactgaaaatcagccgggtgcaggctgaggatgttggactttattactgcatgcaaggtttacaccttccgctcaccttcggccaagggacacgactggagattaaa

SEQ ID NO: 9 - DNA, 375 bp, Artificial sequence
  Synthetic antibody
gaggtgcagctgttggagtctgggggaggcttggtacagcctggggggtccctaagactctcctgtgcagcctctggattcacctttaacaactctgccatgggcagttatgccatgatctgggtccgccaggctccagggaaggggctggagtgggtctcaactattaccggtactggtcttaccacatactacgcagactccgtgaagggccggttcaccgtctccagagacaattccaggaacacgctgcatctgcaaatgaacagcctgagagccgaggacacggccgtctattactgtgcgaaatggaatataattactatggccccttttgatatctggggccaagggacattggtcaccgtctcttca

SEQ ID NO: 10 - DNA, 330 bp, Artificial sequence
  Synthetic antibody
cagactgtggtgacccaggagccatcgttctcagtgtcccctggagggacagtcacactcacttgtggcttgacctctggctcagtctctactagttactataccagctggtaccagcagaccccaggccaggctccacgcacgctcatctacaagacaaacactcgctcttctggggtccctgatcgcttctctggctccatcgttgggaacaaagctgccctcaccatcacgggggcccagccagatgatgaatctgattattactgtgtgctgtatgtgggtagtggcatttgggtgttcggcggagggaccaagctgaccgtccta

SEQ ID NO: 11 - DNA, 360 bp, Artificial sequence
  Synthetic antibody
caggtgcagctggtgcagtctggggctgaggtgaagaagcctggggcctcagtgaaggtctcctgcaaggcttctggatacaccttcaccggctaccagatgcactgggtgcgacaggcccctggccaagggcttgagtggatgggatggatcaacccttacaccggggacacaagttattcacagaagtttcagggcagggtcaccatgacccgggacacgtccatcaacacagcctacatggagctgaacaggctgcgccctgacgactcggccgtgtattactgtgcgagatacgatttctggagtgttcatatctttgacttgtggggccagggaaccctggtcactgtctcctca

SEQ ID NO: 12 - DNA, 327 bp, Artificial sequence
  Synthetic antibody
gactttgtgttgacgcagtctccaggcaccctgtctttatctccaggggaaagagccaccctctcctgcagggccagtcagagtgttagcagcagcttcttaggctggtaccagcagaaacctggccagcctcccagactcctcatctatggtgcatccagcagggccactggcatcccagacaggttcagtggtagtgggtctgggacagacttcactctcaccatcagcagactggagcctgaagattttgcagtgtattactgtcagcattatggtacctcacctgggttcactttcggccctgggaccaaagtggatatcaaa

SEQ ID NO: 13 - DNA, 360 bp, Artificial sequence; identical to SEQ ID NO: 11
  Synthetic antibody

SEQ ID NO: 14 - DNA, 327 bp, Artificial sequence; identical to SEQ ID NO: 12
  Synthetic antibody

SEQ ID NO: 15 - DNA, 369 bp, Artificial sequence
  Synthetic antibody
caggtgcagctgcaggagtcgggccccggactggtgaagccttcacagaccctgtccctcacctgcactgtctctggtggctccatgatcagtggtcgtttctactggagctggatccggcagcccgccgggaagggactggagtggattgggcgtatatataatagtgggagcaccaattacaacccctccctcaaaagtcgagtcaccttatcactggacacgtccaagaacgagttctccctgaagctgacctctgtgaccgccgcagacacggccgtgtactactgtgcgaaggaggacgatttttggagtggccatggggggttcgacccctggggccagggaaccccggtcaccgtctcctca

SEQ ID NO: 16 - DNA, 318 bp, Artificial sequence
  Synthetic antibody
gaaatagtgatgacgcagtctccagccaccctgtctgtgtctccaggggaaagagtcaccctctcctgcagggccagtcagagtgttcacatcaacgtagcctggtaccaccagaaacctggccaggctcccaggctcctcatctatggtgcatccaaaagggccactggtatcccaggcaggttcagtggcagtgggtctgggacagagttcactctcaccatcagcagcctggagtctgaagattttgcagtgtatttctgtcagcagtataacaactggcccactttcggccctgggacccaggtggatatcaaa

SEQ ID NO: 17 - DNA, 366 bp, Artificial sequence
  Synthetic antibody
gaagtgcagctggtgcagtctggagcagaggtgaaaaagcccggggagtctctgaggatctcctgtaagggttctggatacaactttgccagctactggatcacctgggtgcgccagatgcccgggaaaggcctggagtggatggggaggattgatcctagtgactcttataccaactacagcccgtccttccaaggccacgtcaccatctcagctgacaagtccatcagcactgcctacctgcagtggagcaccctgaaggcctcggacaccgccatgtattactgtgcgcgatcgggatcgttttggagtgcttttaactggttcgacccctggggccagggaaccctggtcaccgtctcctca

SEQ ID NO: 18 - DNA, 330 bp, Artificial sequence
  Synthetic antibody
cagtctgccctgacgcagcctgcctccgtgtctgggtctccaggacagtcgatcaccatctcctgcactgcaaccagcagtgaaattgggagttataaccttgtctcctggtaccaacaacacccaggcaaagcccccaaagtcaagatttatgagggcactaagcggccctcaggggtttcaaatcgcttctctggctccaagtctggcaatacggcctccctgacaatctctgggctccaggctgaggacgaggctgattattactgctgctcatatgcaggtagtaacacttgggtgttcggcggagggaccaagctgaccgtccta

SEQ ID NO: 19 - DNA, 372 bp, Artificial sequence
  Synthetic antibody
gaggtgcaggtggtggagtctgggggaggcttggtccagccgggggggtccctgagactctcctgtgcagcctctggattcacctttagttactattggatgagctgggtccgccaggctccagggaaggggctggagtgggtggccaacataaagcaagatggaagtgagaaaaactatgtggactctgtgaagggccgattcaccatctccagagacaacgccaagaactcactgtatctgcaaatgagcagcctgagagccgaggacacggccgtgtattactgtgcgagaagggattacgctttttggagtggttatcgctctttgtgggactactggggccagggaaccctggtcaccgtctcctca

SEQ ID NO: 20 - DNA, 321 bp, Artificial sequence
  Synthetic antibody
gacatccagatgacccagtctccatcctccctgtctgcatctgtgggagacagagtcaccatcacttgccgggcaaatcaaagcattagtaggtttttgaattggtatcaacacaaaccagggaaagcccctaaggtcctgatctacgctgcttccagtttgcaaagtggtgtcccctcaaggttcagtggcagtcaatctgggacagatttcactctcaccatcagcagtctgcaacctgaagattttggaacttactactgtcaacagagtcacagtcccccggagacgttcggccaagggaccaaggtggaaatcaaa

SEQ ID NO: 21 - DNA, 366 bp, Artificial sequence
  Synthetic antibody
gaaatgcagctgcaggagtcgggcccaagactggtgaagccttcacagaccctgtccctcacctgcactgtctctggtggctccaccagcagtggtggttactactggagctggttccgccagtaccccgagaagggcctggagtggattgggtacatctttagtagtgtgaccacctactacaacccgcccctcaagagtcgagtcaccatatcagtagacacgtctaagaaccagttctccctgaagttgagctctgtcactgccgcggacacggccgtgtattactgtgcgagatctttacattactatcagagtagtggtttcctctactggggccggggaatcccggtcaccgtctccgca

SEQ ID NO: 22 - DNA, 318 bp, Artificial sequence
  Synthetic antibody
gaaattgtgttgacacagtctccagccaccctgtctttgtctccaggggatagagccaccctctcctgcagggccagtcagactgttggcgactccttggcctggtaccaacaaaaacctggccaggctcccaggctcctcatctatgatgtttccaacagggccactggcatcccagccaggttcagtggcagtgggtctgggacagacttcactctcaccatcagcagcctagagcctgaggattttgcagtttattactgtcagcagcgtgccagcttcgtcaccttcggcagagggaccaaggtggacatcaaa

SEQ ID NO: 23 - DNA, 363 bp, Artificial sequence
  Synthetic antibody
caggtgcagctgcaggagtcgggcccaggactggtgaagccttcggagaccctgtccctcacctgcagtgtctctggtgcctccatcaggagtgactactggatctggatccggcagcccgccgggaagggactggagtggattgggcgtattgacaccactgggaagaccaactacaacccctcccttaagagtcgagtcaccatgtcagttgacacgtccaagaaccagttctccctgaagctgaggtctgtgaccgccgcggacacggccgtgtattattgtgcgagagaataccgactacggtggacagtgtactactttgactactggggccagggaaccctggtcaccgtctcctca

SEQ ID NO: 24 - DNA, 324 bp, Artificial sequence
  Synthetic antibody
gaaatagtgatgacgcagtctccagccaccctgtctgtgtctccaggggaaagagccaccctctcttgcagggccagtcagagtgttagcagcaacttagcctggtaccagcagagacctggccaggctcccaggctcctcatctatggtgcatccaccagggccactggtatcccagccaggttcactggcagtgggtctgggacagagttcaccctcaccatcagcagcctgcagtctgaagattttgcagtttattactgtcagcagtatagtgactggtttcagctcactttcggcggagggaccaaggtggagatcaaa

SEQ ID NO: 25 - DNA, 375 bp, Artificial sequence
  Synthetic antibody
caggtgcagctggtggagtctgggggaggcttggtcaagcctggagggtccctgagactctcctgtgcagcctctggattcagcttcagtgacttccacatgagctggatccgtcaggctccagggaaggggctggagtgggtatcatatattagtagtagtagtctttccacaaagtacgcagactctgtgaagggccgattcaccatctccagagacaacgccaagaactcactgtatctgcaaatgaacagcctgagagccgaggacacggccgtgtattactgtgcgagatgggacattattaccatgatagtcgtacttggggatgctgttgatatctggggccaagggacaaaggtcaccgtctcttca

SEQ ID NO: 26 - DNA, 333 bp, Artificial sequence
  Synthetic antibody
cagactgtggtgactcaggagccctcactgactgtgtccccaggagggacagtcactctcacctgcgcttccagcactggagcagtcaccaggactttctatccaaactggttccagcagaaacctggacaagcacccagggcactgatttatagtacaagcaaaaaacactcctggacccctgcccggttctcaggctccctccttgggggcaaagctgccctgacactgtcgggtgtgcagcctgaggacgaggctgagtatcactgcctgctctactattatggtgcccagctttgggtgttcggcggagggaccaagctgaccgtccta

SEQ ID NO: 27 - protein, 122 aa, Artificial sequence
  Synthetic antibody
QVQLVQSGAEVKKPGAPVKVSCEASGYTFTDYFIHWVRQAPGQGLEWMGWINPISGGTNYHPRFHGGVTMTRDTSMKVAYMELKRLTSDDTAVYFCARGRDFRGGYSQLDYWGQGTLVTVSS

SEQ ID NO: 28 - protein, 110 aa, Artificial sequence
  Synthetic antibody
QSVLTQPPSASGTPGQRVTISCSGGSSNIAINTVNWYQQVPGTAPKLLMYSNNQRPSGVPDRFSGSKSGTSASLAISGLQSEDEADYYCATWDDSLKDVLFGGGTKLTVL

SEQ ID NO: 29 - protein, 121 aa, Artificial sequence
  Synthetic antibody
QVQLQESGPGLVKPSETLSLTCTVSGASISSYSWSWIRQPAGRGLEWLGRIYPSGNTNYSPSLKSRLTMSLDTSKNQFSMKLTSVSAADTAVYYCARDREQWPLYYGMDVWGQGTLVTVSS

SEQ ID NO: 30 - protein, 107 aa, Artificial sequence
  Synthetic antibody
EIVLTQSPAILSLSPGDRATLSCRASQSVFTYLAWYQHKPGQAPRLLIYDASNRASGIPARFSGSGSGTDFTLTISSLEPEDFAVYYCQQRTKWPLAFGGGTKVEIK

SEQ ID NO: 31 - protein, 122 aa, Artificial sequence
  Synthetic antibody
QVQLQESGPGLVKPSETLSLSCTVSGGSISPDYWSWIRQPPGKGLEWLGYIYSAGSTSYNPSLKSRVTMSVDTSKNQLSLKLTSVTAADTAVYYCARTAGSFWSGRGWFDPWGQGTLVTVSS

SEQ ID NO: 32 - protein, 114 aa, Artificial sequence
  Synthetic antibody
EIVLTQSPATLSLSPGERVTLSCGASQSVSSSHLAWYQQKPGLAPRLLIYDASNRATGVPDRFSGSGSGTDFTLTISRLEPEDFAVYYCQQYGSPQYTFGQGTKLEIKRTVAAP

SEQ ID NO: 33 - protein, 124 aa, Artificial sequence
  Synthetic antibody
QCQVELVESGGDVVQPGKSLRLSCAASGFTFTNYAMHWLRQAPGKGLEWVAVISSDVNDKYYADSVKGRFTISRDNSKNTLYLQMNSLTPEDTAVYYCAREQAVGTNPWAFDYWGQGTLVTVSS

SEQ ID NO: 34 - protein, 113 aa, Artificial sequence
  Synthetic antibody
HIVMTQSPLSLSVTPGQPASISCKSSQISSWGSDGKTYLYWYLQKPGQSPQLLIYEVSSRFSGVSDRFSGSGSGTDFTLKISRVQAEDVGLYYCMQGLHLPLTFGQGTRLEIK

SEQ ID NO: 35 - protein, 125 aa, Artificial sequence
  Synthetic antibody
EVQLLESGGGLVQPGGSLRLSCAASGFTFNNSAMGSYAMIWVRQAPGKGLEWVSTITGTGLTTYYADSVKGRFTVSRDNSRNTLHLQMNSLRAEDTAVYYCAKWNIITMAPFDIWGQGTLVTVSS

SEQ ID NO: 36 - protein, 110 aa, Artificial sequence
  Synthetic antibody
QTVVTQEPSFSVSPGGTVTLTCGLTSGSVSTSYYTSWYQQTPGQAPRTLIYKTNTRSSGVPDRFSGSIVGNKAALTITGAQPDDESDYYCVLYVGSGIWVFGGGTKLTVL

SEQ ID NO: 37 - protein, 120 aa, Artificial sequence
  Synthetic antibody
QVQLVQSGAEVKKPGASVKVSCKASGYTFTGYQMHWVRQAPGQGLEWMGWINPYTGDTSYSQKFQGRVTMTRDTSINTAYMELNRLRPDDSAVYYCARYDFWSVHIFDLWGQGTLVTVSS

SEQ ID NO: 38 - protein, 109 aa, Artificial sequence
  Synthetic antibody
DFVLTQSPGTLSLSPGERATLSCRASQSVSSSFLGWYQQKPGQPPRLLIYGASSRATGIPDRFSGSGSGTDFTLTISRLEPEDFAVYYCQHYGTSPGFTFGPGTKVDIK

SEQ ID NO: 39 - protein, 120 aa, Artificial sequence; identical to SEQ ID NO: 37
  Synthetic antibody

SEQ ID NO: 40 - protein, 109 aa, Artificial sequence; identical to SEQ ID NO: 38
  Synthetic antibody

SEQ ID NO: 41 - protein, 123 aa, Artificial sequence
  Synthetic antibody
QVQLQESGPGLVKPSQTLSLTCTVSGGSMISGRFYWSWIRQPAGKGLEWIGRIYNSGSTNYNPSLKSRVTLSLDTSKNEFSLKLTSVTAADTAVYYCAKEDDFWSGHGGFDPWGQGTPVTVSS

SEQ ID NO: 42 - protein, 106 aa, Artificial sequence
  Synthetic antibody
EIVMTQSPATLSVSPGERVTLSCRASQSVHINVAWYHQKPGQAPRLLIYGASKRATGIPGRFSGSGSGTEFTLTISSLESEDFAVYFCQQYNNWPTFGPGTQVDIK

SEQ ID NO: 43 - protein, 122 aa, Artificial sequence
  Synthetic antibody
EVQLVQSGAEVKKPGESLRISCKGSGYNFASYWITWVRQMPGKGLEWMGRIDPSDSYTNYSPSFQGHVTISADKSISTAYLQWSTLKASDTAMYYCARSGSFWSAFNWFDPWGQGTLVTVSS

SEQ ID NO: 44 - protein, 110 aa, Artificial sequence
  Synthetic antibody
QSALTQPASVSGSPGQSITISCTATSSEIGSYNLVSWYQQHPGKAPKVKIYEGTKRPSGVSNRFSGSKSGNTASLTISGLQAEDEADYYCCSYAGSNTWVFGGGTKLTVL

SEQ ID NO: 45 - protein, 124 aa, Artificial sequence
  Synthetic antibody
EVQVVESGGGLVQPGGSLRLSCAASGFTFSYYWMSWVRQAPGKGLEWVANIKQDGSEKNYVDSVKGRFTISRDNAKNSLYLQMSSLRAEDTAVYYCARRDYAFWSGYRSLWDYWGQGTLVTVSS

SEQ ID NO: 46 - protein, 107 aa, Artificial sequence
  Synthetic antibody
DIQMTQSPSSLSASVGDRVTITCRANQSISRFLNWYQHKPGKAPKVLIYAASSLQSGVPSRFSGSQSGTDFTLTISSLQPEDFGTYYCQQSHSPPETFGQGTKVEIK

SEQ ID NO: 47 - protein, 122 aa, Artificial sequence
  Synthetic antibody
EMQLQESGPRLVKPSQTLSLTCTVSGGSTSSGGYYWSWFRQYPEKGLEWIGYIFSSVTTYYNPPLKSRVTISVDTSKNQFSLKLSSVTAADTAVYYCARSLHYYQSSGFLYWGRGIPVTVSA

SEQ ID NO: 48 - protein, 106 aa, Artificial sequence
  Synthetic antibody
EIVLTQSPATLSLSPGDRATLSCRASQTVGDSLAWYQQKPGQAPRLLIYDVSNRATGIPARFSGSGSGTDFTLTISSLEPEDFAVYYCQQRASFVTFGRGTKVDIK

SEQ ID NO: 49 - protein, 121 aa, Artificial sequence
  Synthetic antibody
QVQLQESGPGLVKPSETLSLTCSVSGASIRSDYWIWIRQPAGKGLEWIGRIDTTGKTNYNPSLKSRVTMSVDTSKNQFSLKLRSVTAADTAVYYCAREYRLRWTVYYFDYWGQGTLVTVSS

SEQ ID NO: 50 - protein, 108 aa, Artificial sequence
  Synthetic antibody
EIVMTQSPATLSVSPGERATLSCRASQSVSSNLAWYQQRPGQAPRLLIYGASTRATGIPARFTGSGSGTEFTLTISSLQSEDFAVYYCQQYSDWFQLTFGGGTKVEIK

SEQ ID NO: 51 - protein, 125 aa, Artificial sequence
  Synthetic antibody
QVQLVESGGGLVKPGGSLRLSCAASGFSFSDFHMSWIRQAPGKGLEWVSYISSSSLSTKYADSVKGRFTISRDNAKNSLYLQMNSLRAEDTAVYYCARWDIITMIVVLGDAVDIWGQGTKVTVSS

SEQ ID NO: 52 - protein, 111 aa, Artificial sequence
  Synthetic antibody
QTVVTQEPSLTVSPGGTVTLTCASSTGAVTRTFYPNWFQQKPGQAPRALIYSTSKKHSWTPARFSGSLLGGKAALTLSGVQPEDEAEYHCLLYYYGAQLWVFGGGTKLTVL

SEQ ID NO: 53 - protein, 8 aa, Artificial sequence
  Synthetic antibody
GYTFTDYF

SEQ ID NO: 54 - protein, 8 aa, Artificial sequence
  Synthetic antibody
INPISGGT

SEQ ID NO: 55 - protein, 15 aa, Artificial sequence
  Synthetic antibody
ARGRDFRGGYSQLDY

SEQ ID NO: 56 - protein, 8 aa, Artificial sequence
  Synthetic antibody
GASISSYS

SEQ ID NO: 57 - protein, 7 aa, Artificial sequence
  Synthetic antibody
IYPSGNT

SEQ ID NO: 58 - protein, 15 aa, Artificial sequence
  Synthetic antibody
ARDREQWPLYYGMDV

SEQ ID NO: 59 - protein, 8 aa, Artificial sequence
  Synthetic antibody
GGSISPDY

SEQ ID NO: 60 - protein, 7 aa, Artificial sequence
  Synthetic antibody
IYSAGST

SEQ ID NO: 61 - protein, 16 aa, Artificial sequence
  Synthetic antibody
ARTAGSFWSGRGWFDP

SEQ ID NO: 62 - protein, 8 aa, Artificial sequence
  Synthetic antibody
GFTFTNYA

SEQ ID NO: 63 - protein, 8 aa, Artificial sequence
  Synthetic antibody
ISSDVNDK

SEQ ID NO: 64 - protein, 15 aa, Artificial sequence
  Synthetic antibody
AREQAVGTNPWAFDY

SEQ ID NO: 65 - protein, 13 aa, Artificial sequence
  Synthetic antibody
GFTFNNSAMGSYA

SEQ ID NO: 66 - protein, 8 aa, Artificial sequence
  Synthetic antibody
ITGTGLTT

SEQ ID NO: 67 - protein, 13 aa, Artificial sequence
  Synthetic antibody
AKWNIITMAPFDI

SEQ ID NO: 68 - protein, 8 aa, Artificial sequence
  Synthetic antibody
GYTFTGYQ

SEQ ID NO: 69 - protein, 8 aa, Artificial sequence
  Synthetic antibody
INPYTGDT

SEQ ID NO: 70 - protein, 13 aa, Artificial sequence
  Synthetic antibody
ARYDFWSVHIFDL

SEQ ID NO: 71 - protein, 8 aa, Artificial sequence
  Synthetic antibody
GYTFTGYQ

SEQ ID NO: 72 - protein, 8 aa, Artificial sequence
  Synthetic antibody
INPYTGDT

SEQ ID NO: 73 - protein, 13 aa, Artificial sequence
  Synthetic antibody
ARYDFWSVHIFDL

SEQ ID NO: 74 - protein, 10 aa, Artificial sequence
  Synthetic antibody
GGSMISGRFY

SEQ ID NO: 75 - protein, 7 aa, Artificial sequence
  Synthetic antibody
IYNSGST

SEQ ID NO: 76 - protein, 15 aa, Artificial sequence
  Synthetic antibody
AKEDDFWSGHGGFDP

SEQ ID NO: 77 - protein, 8 aa, Artificial sequence
  Synthetic antibody
GYNFASYW

SEQ ID NO: 78 - protein, 8 aa, Artificial sequence
  Synthetic antibody
IDPSDSYT

SEQ ID NO: 79 - protein, 15 aa, Artificial sequence
  Synthetic antibody
ARSGSFWSAFNWFDP

SEQ ID NO: 80 - protein, 8 aa, Artificial sequence
  Synthetic antibody
GFTFSYYW

SEQ ID NO: 81 - protein, 8 aa, Artificial sequence
  Synthetic antibody
IKQDGSEK

SEQ ID NO: 82 - protein, 17 aa, Artificial sequence
  Synthetic antibody
ARRDYAFWSGYRSLWDY

SEQ ID NO: 83 - protein, 10 aa, Artificial sequence
  Synthetic antibody
GGSTSSGGYY

SEQ ID NO: 84 - protein, 7 aa, Artificial sequence
  Synthetic antibody
IFSSVTT

SEQ ID NO: 85 - protein, 14 aa, Artificial sequence
  Synthetic antibody
ARSLHYYQSSGFLY

SEQ ID NO: 86 - protein, 8 aa, Artificial sequence
  Synthetic antibody
GASIRSDY

SEQ ID NO: 87 - protein, 7 aa, Artificial sequence
  Synthetic antibody
IDTTGKT

SEQ ID NO: 88 - protein, 15 aa, Artificial sequence
  Synthetic antibody
AREYRLRWTVYYFDY

SEQ ID NO: 89 - protein, 8 aa, Artificial sequence
  Synthetic antibody
GFSFSDFH

SEQ ID NO: 90 - protein, 8 aa, Artificial sequence
  Synthetic antibody
ISSSSLST

SEQ ID NO: 91 - protein, 18 aa, Artificial sequence
  Synthetic antibody
ARWDIITMIVVLGDAVDI

SEQ ID NO: 92 - protein, 8 aa, Artificial sequence
  Synthetic antibody
SSNIAINT

SEQ ID NO: 93 - protein, 3 aa, Artificial sequence
  Synthetic antibody
SNN

SEQ ID NO: 94 - protein, 11 aa, Artificial sequence
  Synthetic antibody
ATWDDSLKDVL

SEQ ID NO: 95 - protein, 6 aa, Artificial sequence
  Synthetic antibody
QSVFTY

SEQ ID NO: 96 - protein, 3 aa, Artificial sequence
  Synthetic antibody
DAS

SEQ ID NO: 97 - protein, 9 aa, Artificial sequence
  Synthetic antibody
QQRTKWPLA

SEQ ID NO: 98 - protein, 7 aa, Artificial sequence
  Synthetic antibody
QSVSSSH

SEQ ID NO: 99 - protein, 3 aa, Artificial sequence
  Synthetic antibody
DAS

SEQ ID NO: 100 - protein, 9 aa, Artificial sequence
  Synthetic antibody
QQYGSPQYT

SEQ ID NO: 101 - protein, 12 aa, Artificial sequence
  Synthetic antibody
QISSWGSDGKTY

SEQ ID NO: 102 - protein, 3 aa, Artificial sequence
  Synthetic antibody
EVS

SEQ ID NO: 103 - protein, 9 aa, Artificial sequence
  Synthetic antibody
MQGLHLPLT

SEQ ID NO: 104 - protein, 9 aa, Artificial sequence
  Synthetic antibody
SGSVSTSYY

SEQ ID NO: 105 - protein, 3 aa, Artificial sequence
  Synthetic antibody
KTN

SEQ ID NO: 106 - protein, 10 aa, Artificial sequence
  Synthetic antibody
VLYVGSGIWV

SEQ ID NO: 107 - protein, 7 aa, Artificial sequence
  Synthetic antibody
QSVSSSF

SEQ ID NO: 108 - protein, 3 aa, Artificial sequence
  Synthetic antibody
GAS

SEQ ID NO: 109 - protein, 10 aa, Artificial sequence
  Synthetic antibody
QHYGTSPGFT

SEQ ID NO: 110 - protein, 7 aa, Artificial sequence
  Synthetic antibody
QSVSSSF

SEQ ID NO: 111 - protein, 3 aa, Artificial sequence
  Synthetic antibody
GAS

SEQ ID NO: 112 - protein, 10 aa, Artificial sequence
  Synthetic antibody
QHYGTSPGFT

SEQ ID NO: 113 - protein, 6 aa, Artificial sequence
  Synthetic antibody
QSVHIN

SEQ ID NO: 114 - protein, 3 aa, Artificial sequence
  Synthetic antibody
GAS

SEQ ID NO: 115 - protein, 8 aa, Artificial sequence
  Synthetic antibody
QQYNNWPT

SEQ ID NO: 116 - protein, 9 aa, Artificial sequence
  Synthetic antibody
SSEIGSYNL

SEQ ID NO: 117 - protein, 3 aa, Artificial sequence
  Synthetic antibody
EGT

SEQ ID NO: 118 - protein, 10 aa, Artificial sequence
  Synthetic antibody
CSYAGSNTWV

SEQ ID NO: 119 - protein, 6 aa, Artificial sequence
  Synthetic antibody
QSISRF

SEQ ID NO: 120 - protein, 3 aa, Artificial sequence
  Synthetic antibody
AAS

SEQ ID NO: 121 - protein, 9 aa, Artificial sequence
  Synthetic antibody
QQSHSPPET

SEQ ID NO: 122 - protein, 6 aa, Artificial sequence
  Synthetic antibody
QTVGDS

SEQ ID NO: 123 - protein, 3 aa, Artificial sequence
  Synthetic antibody
DVS

SEQ ID NO: 124 - protein, 8 aa, Artificial sequence
  Synthetic antibody
QQRASFVT

SEQ ID NO: 125 - protein, 6 aa, Artificial sequence
  Synthetic antibody
QSVSSN

SEQ ID NO: 126 - protein, 3 aa, Artificial sequence
  Synthetic antibody
GAS

SEQ ID NO: 127 - protein, 10 aa, Artificial sequence
  Synthetic antibody
QQYSDWFQLT

SEQ ID NO: 128 - protein, 9 aa, Artificial sequence
  Synthetic antibody
TGAVTRTFY

SEQ ID NO: 129 - protein, 3 aa, Artificial sequence
  Synthetic antibody
STS

SEQ ID NO: 130 - protein, 11 aa, Artificial sequence
  Synthetic antibody
LLYYYGAQLWV

(19) United States
(12) Patent Application Publication
(10) Pub. No.: US 2023/0078330 A1
(43) Pub. Date: Mar. 16, 2023

(54) HUMAN ANTI-DENGUE ANTIBODIES AND METHODS OF USE THEREFOR

(71) Applicants: VANDERBILT UNIVERSITY, Nashville, TN (US); THE UNIVERSITY OF NORTH CAROLINA AT CHAPEL HILL, Chapel Hill, NC (US); THE REGEBTS OF THE UNIVERSITY OF CALIFORNIA A CALIFORNIA, Oakland, CA (US)

(72) Inventors: James E. CROWE, JR., Nashville, TN (US); Ralph BARIC, Haw River, NC (US); Eva HARRIS, Berkeley, CA (US)

(21) Appl. No.: 17/759,479

(22) PCT Filed: Jan. 19, 2021
(86) PCT No.: PCT/US2021/013903

§ 371 Date: Jul. 26, 2022

Related U.S. Application Data
(60) Provisional application No. 62/966,297, filed on Jan. 27, 2020.

(51) Int. Cl. C07K 16/10; G01N 33/569; A61P 31/14

(52) U.S. Cl. CPC C07K 16/1081; G01N 33/56983; A61P 31/14; G01N 2333/185; G01N 2469/10; G01N 2800/26; C07K 2317/21; C07K 2317/76; A61K 2039/505

(57) ABSTRACT

The present disclosure is directed to antibodies binding to and neutralizing dengue virus and methods for use thereof.

### PRIORITY CLAIM

This application claims benefit of priority from U.S. Provisional Application Ser. No. 62/966,297, filed Jan. 27, 2020, the entire contents of which are hereby incorporated by reference.

### BACKGROUND

This invention was made with Government support under National Institutes of Health Grant No. P01 AI106695 from the National Institute of Allergy and Infectious Diseases, National Institutes of Health Grant UL1 RR024975-01 from the National Center for Research Resources, National Institutes of Health Grant No. 2 UL1 TR000445-06 from the National Center for Advancing Translational Sciences, National Institutes of Health Grant No P30 CA68485 from the National Cancer Institute, and National Institutes of Health Grant No DK058404 from the National Institute of Diabetes and Digestive and Kidney Diseases. The government has certain rights in the invention.

### 1. Field of the Disclosure

The present disclosure relates generally to the fields of medicine, infectious disease, and immunology. More particular, the disclosure relates to human antibodies binding to dengue virus and method of their use for diagnosing and treating dengue virus infections.

### 2. Background

Dengue viruses (DENV) are positive-sense RNA viruses belonging to the Flavivirus genus and are transmitted to humans by Aedes aegypti or Aedes albopictus mosquitoes (de Silva and Harris, 2018; Diamond and Pierson, 2015). It is estimated that the 4 serotypes of DENV (DENV1-4) are responsible for up to 390 million infections and 100 million cases each year (Bhatt et al., 2013), ranging from mild fever to severe Dengue Hemorrhagic Fever and Dengue Shock Syndrome (Halstead et al., 1973). Complicating vaccine design, infection with one DENV serotype does not confer lasting protective immunity to the other three serotypes. After a primary infection, type-specific (TS) antibodies to the infection serotype are associated with durable, essentially life-long, protection (de Alwis et al., 2012; Murphy and Whitehead, 2011). Although cross-reactive (CR) antibodies to the other three serotypes develop during a primary infection, these responses oftentimes wane over time in the absence of secondary exposures, and low to intermediate levels of CR antibodies may contribute to enhanced viral replication and an increased risk of severe disease in some settings (de Alwis et al., 2014; Katzelnick et al., 2017a; Salje et al., 2018; Sangkawibha et al., 1984). Hence, despite the induction of robust TS immunity, an individual with a single previous DENV infection may remain susceptible to developing severe forms of disease during a secondary infection with virus from a heterologous serotype (Halstead, 2015). Following a secondary infection, individuals who recover typically have durable serotype cross-protective immunity (Dejnirattisai et al., 2015a; Patel et al., 2017). The only licensed Dengue vaccine, Denvaxia, caused increased risk in dengue-naïve children for severe Dengue after infection and breakthrough infections with DENV3 were common(Ferguson et al. , 2016). Another tetravalent vaccine, TAK-003 did not protect against DENV3 as the percent efficacy was negative 37% for DENV3 at 18 months in naïve populations(Biswal et al., 2019). The basis for DENV3 vaccine failure is uncertain, however, the full repertoire of antibodies and the epitopes targeted following primary or secondary DENV infections remains only partially characterized, preventing a full understanding of the mechanisms of protective immunity and immune enhancement (Gallichotte et al., 2018a; Katzelnick et al., 2017b).

The DENV envelope (E) glycoprotein mediates viral binding and entry into cells and is the main target of neutralizing antibodies after infection and vaccination (Kuhn et al., 2015; Pierson and Diamond, 2008). The four DENV serotypes vary by 25 to 40% in the amino acid sequence of the E protein (Fleith et al. , 2016). Within each serotype, the E protein sequence of different genotypes varies by 6 to 9% (Chen and Vasilakis, 2011; Flipse and Smit, 2015), and genotypic variation plays an underappreciated role in antibody immune escape (Brien et al. , 2010; Sukupolvi-Petty et al., 2013; Wahala et al., 2010). The DENV E protein consists of three domains (designated E protein domain I (EDI), EDII, and EDIII), and two protomers form head-to-tail dimers on the surface of viral particles. Three dimers lie parallel to each other and form thirty rafts in a herringbone pattern on the mature virion (Fibriansah et al., 2015a). A few human TS neutralizing antibodies against DENV1, DENV2, DENV3 or DENV4 have been mapped, many of which recognize quaternary epitopes that span different E protein molecules and are therefore only present on the assembled virion (de Alwis et al. , 2012; Fibriansah et al. , 2015a; Teoh et al., 2012). The human antibody response to DENV3 has been less studied at the clonal level than the other DENV serotypes. A single potent TS neutralizing human monoclonal antibody (hmAb, 5J7) was described in detail, which recognizes a complex quaternary epitope spanning across three E protomers in viral particles. Using viral reverse genetics, it was demonstrated previously that residues in the DENV3-specific hmAb 5J7 epitope can be transplanted on to the E protein from DENV1 or DENV4 to generate chimeric infectious virions displaying the 5J7 epitope (Andrade et al., 2017; Messer et al., 2016; Widman et al., 2017). Interrogation of these chimeric viruses with panels of hmAbs and primary sera revealed that a highly variable fraction of the polyclonal serum DENV3-reactive neutralizing antibody response targets the hmAb 5J7 epitope, suggesting that major neutralizing epitopes of DENV3 remained undiscovered.

### SUMMARY

Thus, in accordance with the present disclosure, there is provided a method of detecting a dengue virus infection in a subject comprising (a) contacting a sample from said subject with an antibody or antibody fragment having clone-paired heavy and light chain CDR sequences from Tables 3 and 4, respectively; and (b) detecting dengue virus in said sample by binding of said antibody or antibody fragment to a Dengue virus antigen in said sample. The sample may be a body fluid, such as blood, sputum, tears, saliva, mucous or serum, semen, cervical or vaginal secretions, amniotic fluid, placental tissues, urine, exudate, transudate, tissue scrapings or feces. Detection may comprise ELISA, RIA, lateral flow assay or western blot. The method may further comprise performing steps (a) and (b) a second time and determining a change in dengue virus antigen levels as compared to the first assay. The antibody or antibody fragment may be encoded by clone-paired variable sequences as set forth in Table 1, by light and heavy chain variable sequences having 70%, 80%, or 90% identity to clone-paired variable sequences as set forth in Table 1, or by light and heavy chain variable sequences having 95% identity to clone-paired sequences as set forth in Table 1. The antibody or antibody fragment may comprise light and heavy chain variable sequences according to clone-paired sequences from Table 2, light and heavy chain variable sequences having 70%, 80% or 90% identity to clone-paired sequences from Table 2, or light and heavy chain variable sequences having 95% identity to clone-paired sequences from Table 2. The antibody fragment may be a recombinant scFv (single chain fragment variable) antibody, Fab fragment, F(ab′)₂ fragment, or Fv fragment.

In another embodiment, there is provided a method of treating a subject infected with dengue virus or reducing the likelihood of infection of a subject at risk of contracting dengue virus comprising delivering to said subject an antibody or antibody fragment having clone-paired heavy and light chain CDR sequences from Tables 3 and 4, respectively. The antibody or antibody fragment may be encoded by clone-paired variable sequences as set forth in Table 1, by light and heavy chain variable sequences having 70%, 80%, or 90% identity to clone-paired variable sequences as set forth in Table 1, or by light and heavy chain variable sequences having 95% identity to clone-paired sequences as set forth in Table 1. The antibody or antibody fragment may comprise light and heavy chain variable sequences according to clone-paired sequences from Table 2, light and heavy chain variable sequences having 70%, 80% or 90% identity to clone-paired sequences from Table 2, or light and heavy chain variable sequences having 95% identity to clone-paired sequences from Table 2. The antibody fragment may be a recombinant scFv (single chain fragment variable) antibody, Fab fragment, F(ab′)₂ fragment, or Fv fragment. The antibody may be an IgG, or a recombinant IgG antibody or antibody fragment comprising an Fc portion mutated to alter (eliminate or enhance) FcR or FcRn interactions, to increase half-life and/or increase therapeutic efficacy, such as a LALA, LALA PG, N297, GASD/ALIE, DHS, YTE or LS mutation or glycan modified to alter (eliminate or enhance) FcR or FcRn interactions such as enzymatic or chemical addition or removal of glycans or expression in a cell line engineered with a defined glycosylating pattern. The antibody may be a chimeric antibody or a bispecific antibody.

The antibody or antibody fragment may be administered prior to infection or after infection. The subject may be a pregnant female, a sexually active female, or a female undergoing fertility treatments. Delivering may comprise antibody or antibody fragment administration, or genetic delivery with an RNA or DNA sequence or vector encoding the antibody or antibody fragment.

In yet another embodiment, there is provided a monoclonal antibody, wherein the antibody or antibody fragment is characterized by clone-paired heavy and light chain CDR sequences from Tables 3 and 4, respectively. The antibody or antibody fragment may be encoded by clone-paired variable sequences as set forth in Table 1, by light and heavy chain variable sequences having 70%, 80%, or 90% identity to clone-paired variable sequences as set forth in Table 1, or by light and heavy chain variable sequences having 95% identity to clone-paired sequences as set forth in Table 1. The antibody or antibody fragment may comprise light and heavy chain variable sequences according to clone-paired sequences from Table 2, light and heavy chain variable sequences having 70%, 80% or 90% identity to clone-paired sequences from Table 2, or light and heavy chain variable sequences having 95% identity to clone-paired sequences from Table 2. The antibody fragment may be a recombinant scFv (single chain fragment variable) antibody, Fab fragment, F(ab′)₂ fragment, or Fv fragment. The antibody may be an IgG, or a recombinant IgG antibody or antibody fragment comprising an Fc portion mutated to alter (eliminate or enhance) FcR or FcRn interactions, to increase half-life and/or increase therapeutic efficacy, such as a LALA, LALA PG, N297, GASD/ALIE, DHS, YTE or LS mutation or glycan modified to alter (eliminate or enhance) FcR or FcRn interactions such as enzymatic or chemical addition or removal of glycans or expression in a cell line engineered with a defined glycosylating pattern. The antibody may be a chimeric antibody or a bispecific antibody. The antibody or antibody fragment may further comprise a cell penetrating peptide and/or is an intrabody.

In still yet another embodiment, there is provided a hybridoma or engineered cell encoding an antibody or antibody fragment wherein the antibody or antibody fragment is characterized by clone-paired heavy and light chain CDR sequences from Tables 3 and 4, respectively. The antibody or antibody fragment may be encoded by clone-paired variable sequences as set forth in Table 1, by light and heavy chain variable sequences having 70%, 80%, or 90% identity to clone-paired variable sequences as set forth in Table 1, or by light and heavy chain variable sequences having 95% identity to clone-paired sequences as set forth in Table 1. The antibody or antibody fragment may comprise light and heavy chain variable sequences according to clone-paired sequences from Table 2, light and heavy chain variable sequences having 70%, 80% or 90% identity to clone-paired sequences from Table 2, or light and heavy chain variable sequences having 95% identity to clone-paired sequences from Table 2. The antibody fragment may be a recombinant scFv (single chain fragment variable) antibody, Fab fragment, F(ab′)₂ fragment, or Fv fragment. The antibody may be an IgG, or a recombinant IgG antibody or antibody fragment comprising an Fc portion mutated to alter (eliminate or enhance) FcR or FcRn interactions, to increase half-life and/or increase therapeutic efficacy, such as a LALA, LALA PG, N297, GASD/ALIE, DHS, YTE or LS mutation or glycan modified to alter (eliminate or enhance) FcR or FcRn interactions such as enzymatic or chemical addition or removal of glycans or expression in a cell line engineered with a defined glycosylating pattern. The antibody may be a chimeric antibody or a bispecific antibody. The antibody or antibody fragment may further comprise a cell penetrating peptide and/or is an intrabody.

In a further embodiment, there is provided a vaccine formulation comprising one or more antibodies or antibody fragments characterized by clone-paired heavy and light chain CDR sequences from Tables 3 and 4, respectively. The at least one antibody or antibody fragment may be encoded by light and heavy chain variable sequences according to clone-paired sequences from Table 1, by light and heavy chain variable sequences having at least 70%, 80%, or 90% identity to clone-paired sequences from Table 1, or by light and heavy chain variable sequences having at least 95% identity to clone-paired sequences from Table 1. The at least one antibody or antibody fragment may comprise light and heavy chain variable sequences according to clone-paired sequences from Table 2, or light and heavy chain variable sequences having 95% identity to clone-paired sequences from Table 2. The at least one antibody fragments may be a recombinant scFv (single chain fragment variable) antibody, Fab fragment, F(ab′)₂ fragment, or Fv fragment. The at least one antibody may be a chimeric antibody or is bispecific antibody. The least one antibodies may be an IgG, or a recombinant IgG antibody or antibody fragment comprising an Fc portion mutated to alter (eliminate or enhance) FcR or FcRn interactions, to increase half-life and/or increase therapeutic efficacy, such as a LALA, LALA PG, N297, GASD/ALIE, DHS, or LS mutation or glycan modified to alter (eliminate or enhance) FcR or FcRn interactions such as enzymatic or chemical addition or removal of glycans or expression in a cell line engineered with a defined glycosylating pattern. The at least one antibody or antibody fragment may further comprise a cell penetrating peptide and/or is an intrabody.

In yet a further embodiment, there is provided a vaccine formulation comprising one or more expression vectors encoding a first antibody or antibody fragment as described herein.

The expression vector(s) may be Sindbis virus or VEE vector(s). The vaccine may be formulated for delivery by needle injection, jet injection, or electroporation. The vaccine may further comprise one or more expression vectors encoding for a second antibody or antibody fragment, such as a distinct antibody or antibody fragment of claims 26-34.

A method of protecting the health of a placenta and/or fetus of a pregnant a subject infected with or at risk of infection with dengue virus comprising delivering to said subject an antibody or antibody fragment having clone-paired heavy and light chain CDR sequences from Tables 3 and 4, respectively. The antibody or antibody fragment may be encoded by clone-paired variable sequences as set forth in Table 1, by light and heavy chain variable sequences having 70%, 80%, or 90% identity to clone-paired variable sequences as set forth in Table 1, or by light and heavy chain variable sequences having 95% identity to clone-paired sequences as set forth in Table 1. The antibody or antibody fragment may comprise light and heavy chain variable sequences according to clone-paired sequences from Table 2, light and heavy chain variable sequences having 70%, 80% or 90% identity to clone-paired sequences from Table 2, or light and heavy chain variable sequences having 95% identity to clone-paired sequences from Table 2. The antibody fragment may be a recombinant scFv (single chain fragment variable) antibody, Fab fragment, F(ab′)₂ fragment, or Fv fragment. The antibody may be an IgG, or a recombinant IgG antibody or antibody fragment comprising an Fc portion mutated to alter (eliminate or enhance) FcR or FcRn interactions, to increase half-life and/or increase therapeutic efficacy, such as a LALA, LALA PG, N297, GASD/ALIE, DHS, YTE or LS mutation or glycan modified to alter (eliminate or enhance) FcR or FcRn interactions such as enzymatic or chemical addition or removal of glycans or expression in a cell line engineered with a defined glycosylating pattern. The antibody may be a chimeric antibody or a bispecific antibody.

The antibody or antibody fragment may be administered prior to infection or after infection. The subject is a pregnant female, a sexually active female, or a female undergoing fertility treatments. Delivering may comprise antibody or antibody fragment administration, or genetic delivery with an RNA or DNA sequence or vector encoding the antibody or antibody fragment. The antibody or antibody fragment may increase the size of the placenta as compared to an untreated control. The antibody or antibody fragment may reduce viral load and/or pathology of the fetus as compared to an untreated control.

In still an additional embodiment, there is provided a method of determining the antigenic integrity, correct conformation and/or correct sequence of a Dengue virus antigen comprising (a) contacting a sample comprising said antigen with a first antibody or antibody fragment having clone-paired heavy and light chain CDR sequences from Tables 3 and 4, respectively; and (b) determining antigenic integrity, correct conformation and/or correct sequence of said antigen by detectable binding of said first antibody or antibody fragment to said antigen. The sample may comprise recombinantly produced antigen or a vaccine formulation or vaccine production batch. Detection may comprise ELISA, RIA, western blot, a biosensor using surface plasmon resonance or biolayer interferometry, or flow cytometric staining.

The first antibody or antibody fragment may be encoded by clone-paired variable sequences as set forth in Table 1, by light and heavy chain variable sequences having 70%, 80%, or 90% identity to clone-paired variable sequences as set forth in Table 1, or by light and heavy chain variable sequences having 95% identity to clone-paired sequences as set forth in Table 1. The first antibody or antibody fragment may comprise light and heavy chain variable sequences according to clone-paired sequences from Table 2, light and heavy chain variable sequences having 70%, 80% or 90% identity to clone-paired sequences from Table 2, or light and heavy chain variable sequences having 95% identity to clone-paired sequences from Table 2. The antibody fragment may be a recombinant scFv (single chain fragment variable) antibody, Fab fragment, F(ab′)₂ fragment, or Fv fragment. The method may further comprise performing steps (a) and (b) a second time to determine the antigenic stability of the antigen over time.

The may further comprise (c) contacting a sample comprising said antigen with a second antibody or antibody fragment having clone-paired heavy and light chain CDR sequences from Tables 3 and 4, respectively; and (d) determining antigenic integrity of said antigen by detectable binding of said second antibody or antibody fragment to said antigen. The antibody or antibody fragment may be encoded by clone-paired variable sequences as set forth in Table 1, by light and heavy chain variable sequences having 70%, 80%, or 90% identity to clone-paired variable sequences as set forth in Table 1, or by light and heavy chain variable sequences having 95% identity to clone-paired sequences as set forth in Table 1. The antibody or antibody fragment may comprise light and heavy chain variable sequences according to clone-paired sequences from Table 2, light and heavy chain variable sequences having 70%, 80% or 90% identity to clone-paired sequences from Table 2, or light and heavy chain variable sequences having 95% identity to clone-paired sequences from Table 2. The antibody fragment may be a recombinant scFv (single chain fragment variable) antibody, Fab fragment, F(ab′)₂ fragment, or Fv fragment. The method may further comprise performing steps (c) and (d) a second time to determine the antigenic stability of the antigen over time.

Also provided is a human monoclonal antibody or antibody fragment, or hybridoma or engineered cell producing the same, wherein said antibody binds to Dengue virus serotype 3 and does not bind to other Dengue virus serotypes, or a human monoclonal antibody or antibody fragment, or hybridoma or engineered cell producing the same, wherein said antibody binds in a serotype 3-specific manner to an epitope in dengue virus type 3 E glycoprotein domain I, or an epitope in domain II, or a quaternary epitope comprising residues in domains I and II.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-E. Fifteen hmAbs isolated from 3 children post DENV3 infection are DENV3-specific. Memory B cells were isolated and immortalized from children experiencing primary or secondary DENV3 infections in Nicaragua. Type specific hmAbs for isolated that bound and neutralized DENV3. (FIG. 1A) DENV immune PBMCs from children in a Nicaraguan cohort study experienced DENV3 as either a primary infection or secondary infection after a DENV2 primary infection. (FIG. 1B) Fifteen hmAbs from Nicaraguan cohort were tested against DENV1-4 by Vero-81 Focus Reduction Neutralization Test (FRNT). Full curves are shown. DENV1-specific hmAbs 1F4 and 14c10, DENV2-specific hmAb 2D22, DENV3-specific hmAb 5J7 and a recombinant form of the cross-reactive hmAb EDEI C8 were used as positive controls. All assays were performed twice in duplicate. (FIG. 1C) DENV 3 neutralization by hmAbs was evaluated by standard micro-neutralization assays using wildtype and recombinant viruses. Comparison of DENV3-specific hmAbs to 5J7. Using wildtype DENV3, Vero-81 cell FRNT EC₅₀ values from repeat experiments performed on different days were averaged to show reproducibility. EC₅₀ value denotes amount of hmAb needed to neutralize 50% of the virus in a Vero-81 FRNT. Error bars indicate standard deviation. (FIG. 1D) DENV4/3 M16 ic—PyMOL representation of DENV3 residues (blue) that capture the hmAb 5J7 epitope transplanted into a DENV4 backbone. (FIG. 1E) DENV3-specific hmAbs do not use 5J7 epitope. DENV4/3 M16 ic is neutralized by hmAb 5J7 but not by the panel of 15 DENV3-specific hmAbs. EC₅₀ values in a FRNT of hmAbs of DENV4/3 M16 ic, and parental DENV3 ic and DENV4ic.

FIGS. 2A-D. DENV3/1 recombinant EDI loss-of-function chimeras reveal new DENV3 hmAb characteristics. A panel of chimeric DENV3 viruses encoding progressively larger blocks of DENV1 E glycoprotein sequence was used to interrogate the role of these transplanted regions in loss of DENV3 antibody function. (FIG. 2A) Four DENV3/1 chimeric viruses with increasing portions of DENV1 residues in a DENV3 backbone. DENV3/1 chimera PyMOL representations of DENV1 residues (orange) transplanted into DENV3 backbone (grey). Number of amino acids changed is in parenthesis. (FIG. 2B) Amino acid alignment of changed residues in DENV3/1 chimeras. DENV3 residues are shown in blue. DENV1 residues are shown in orange. A tissue culture adaptation in DENV3/1 A is shown in yellow. Blank spaces in DENV3 indicate residues not present in DENV1. (FIG. 2C) DENV3/1 chimeras contain DENV1-specific, DENV3-specific and cross-reactive epitopes. EC₅₀ values of Vero-81 cell FRNT of DENV3-specific hmAb 5J7, cross-reactive hmAb EDEI C8 and DENV1-specific hmAbs 1F4 and 14c10. (FIG. 2D) Panel of 15 hmAbs can be sorted into 3 groups by EC₅₀ values of Vero-81 cell FRNT against chimeric DENV3/1 viruses. Group 1: Ten hmAbs do not neutralize any members of the chimeric dengue 3/1 panel. Group 2: hmAbs DENV-115, DENV-290 and -419 neutralize all chimeric DENV3/1 viruses (red circles). Group 3: hmAbs DENV-66 and -144 neutralize only DENV3/1 EDI-A and -B (blue arrows).

FIGS. 3A-D. DENV1/3 EDI shows gain of function for 9 of 10 DENV3 hmAbs in group I, mapping to EDI. To interrogate gain of neutralization function, DENV1/3 EDI chimeric viruses with increased numbers of EDI residues from DENV3 introduced into the DENV1 backbone were constructed. PyMOL software-generated representations of changed residues in the DENV1/3 EDI-A chimera. Transplanted DENV1 residues are shown in orange spheres on a DENV3 backbone. Top and side views are shown. (FIG. 3A) DENV1/3 EDI-A chimera surface residues were changed. (FIG. 3B) DENV1/3 EDI-B chimera surface and interior residues were changed. (FIG. 3C) Amino acid alignment of changed residues in DENV1/3 EDI chimeras. DENV3 residues are shown in blue. DENV1 residues are shown in orange. A tissue culture adaptation in DENV3/1-A is shown in yellow. Blank spaces in DENV3 indicate residues not present in DENV1. (FIG. 3D) EC₅₀ values of Vero-81 cell FRNT of hmAbs against chimeric DENV1/3 viruses. 9 of 10 group 1 hmAbs neutralized both chimeric DENV1/3 viruses. DENV1/3 EDI-B neutralization pattern is most similar to that of DENV3. As expected 1F4, 14c10 and 5J7 do not neutralize either DENV1/3 chimera.

FIGS. 4A-E. DENV3 genotype panel. To identify natural variation that disrupts hmAb function, a panel of recombinant viruses were used that encoded E glycoproteins derived from the different DENV3 genotypes. Subsequently, DENV3 genotype chimeras were used to map hmAbs to EDI, EDII or EDIII in the E glycoprotein by gain-of-function or loss-of-function. (FIG. 4A) DENV3 E glycoprotein dimers for designated GI-IV are shown. Amino acid residues that differ from those in the Sri Lanka genotype III are shown as spheres. A black sphere indicates the residue is unique to that genotype and not shared between the genotypes. Colored spheres indicate residues seen in two or more genotypes. (FIG. 4B) Amino acid alignment. Amino acids with nonpolar side chains are colored orange, uncharged polar are green, acidic are red and basic are blue. Domains are indicated in gray bar at bottom. (FIG. 4C) Genotypic variation alters FRNT neutralization EC₅₀ values for select DENV3 hmAbs. Genotype IV DENV3 has the greatest amino acid variation and escaped neutralization by some DENV3 hmAbs. (FIG. 4D) Gain-of-function genotype IV DENV3 chimeric viruses with EDI, EDII, or EDIII from genotype III DENV3 were used to map select hmAbs to specific domains of E glycoprotein (see FIGS. S5A-B). EC₅₀ values of Vero-81 cell FRNT for select hmAbs show gain-of-function for DENV-236, -297 and -415 when EDI is transplanted. DENV-115 and -419 show gain-of-function when EDII is transplanted. DENV-66 shows gain-of-function when EDIII is transplanted. (FIG. 4E) Loss-of-function genotype III DENV3 chimeric viruses with EDI, EDII, or EDIII from genotype IIV DENV3. EC₅₀ values of Vero-81 cell FRNT for select hmAbs. DENV-236, -297 and -415 show loss-of-function when EDI is transplanted. DENV-115 and -419 show loss-of-function when EDII is transplanted. DENV-66 shows loss-of-function when EDIII is transplanted.

FIG. 5. Reduction of DENV3 viral burden in vivo by selected hmAbs. AG129 mice were administered 50 μg hmAb (unless otherwise indicated) by intraperitoneal injection 24 hours prior to infection with 5×10⁶ pfu of DENV3 UNC3009. Virus titers were assessed 72 hours post-infection using quantitative RT-PCR of RNA isolated from the spleens of infected mice and are expressed as genome equivalents (GE) normalized to μg of GAPDH. Group 1 DENV-443 and Group 2 DENV-115, -290, and -419 hmAbs reduced DENV3 viral load compared to IgG isotype antibody. The number of mice in each treatment group are indicated, comprising 5 independent experiments in total, with at least 2 independent experiments performed for each hmAb. The limit of detection is 10⁴ GE/μg of GAPDH. Comparisons were performed using Kruskal-Wallis test with Dunn's multiple comparisons (**=p<0.005, ***=p<0.0005, ****=p<0.0001).

FIGS. 6A-B. Six distinct neutralizing epitopes defined by panel of 15 DENV3 hmAbs. Six distinct functional type specific neutralizing epitopes in DENV3 were identified. (FIG. 6A) Summary of antibody properties, phenotypes and interaction sites using GOF DENV1/3 or LOF DENV3/1 chimeras, DENV3 genotype variants and DENV3 genotype chimeras. (FIG. 6B) Shown is a ribbon diagram of a DENV3 E trimer with EDI in red, EDII in yellow and EDIII in blue. Predicted functional epitope sites are shown as black circles. hmAbs in Group 1a interface with EDI differently from Group 1b, while Group 1c interacts in a more complex manner The epitope for DENV-144 has the highest degree of uncertainty. HmAbs from secondary DENV3 infection are in purple squares.

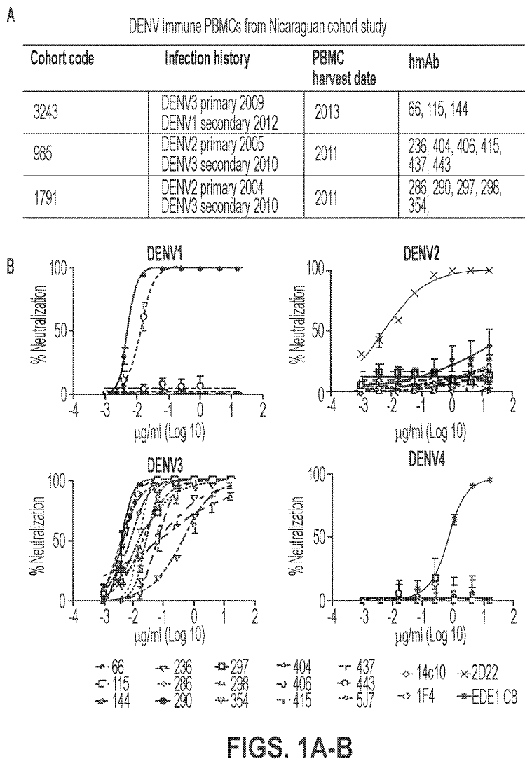

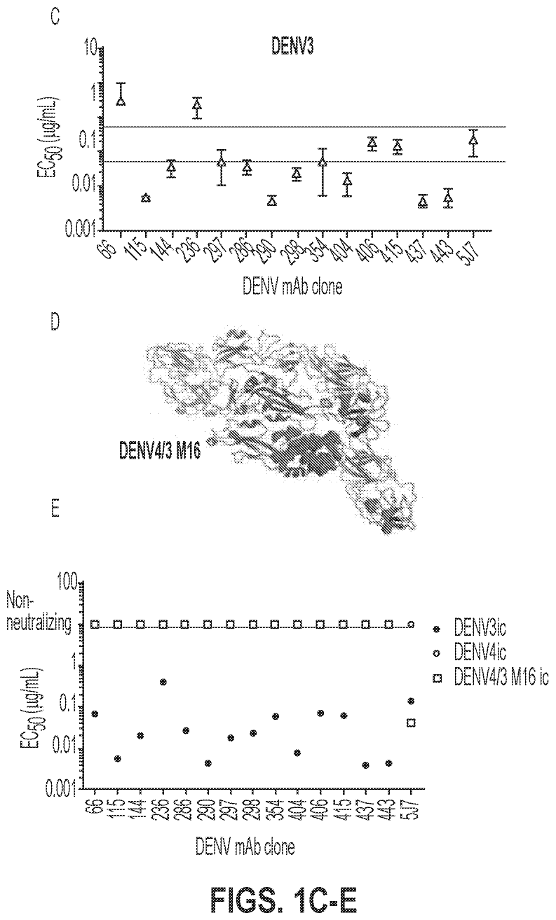

FIG. S1. DENV3 hmAbs are type-specific by capture ELISA. Related to FIGS. 1A-E and Tables A-a and A-b. Binding curves of individual hmAbs to 4G2 captured DENV1, DENV2, DENV3 or DEMV4 are shown. DENV Serotype mAb are DENV1-specific hmAb 1F4, DENV2-specific hmAb 2D22, DENV3-specific hmAb 5J7 and DENV4-specific hmAb D4-126 and were used as positive controls.

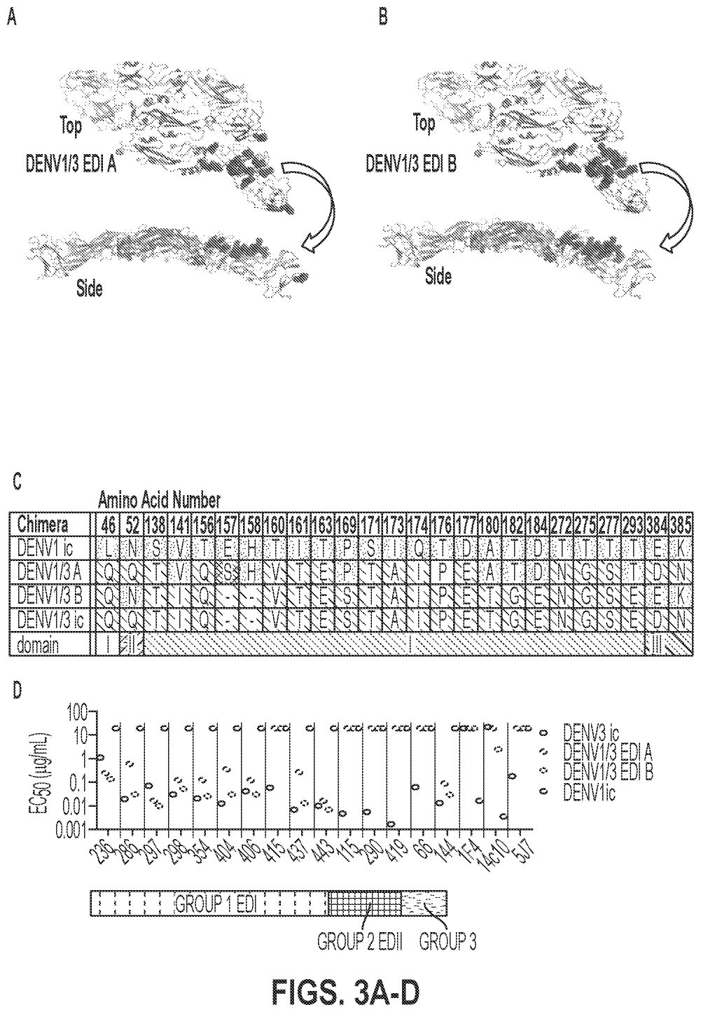

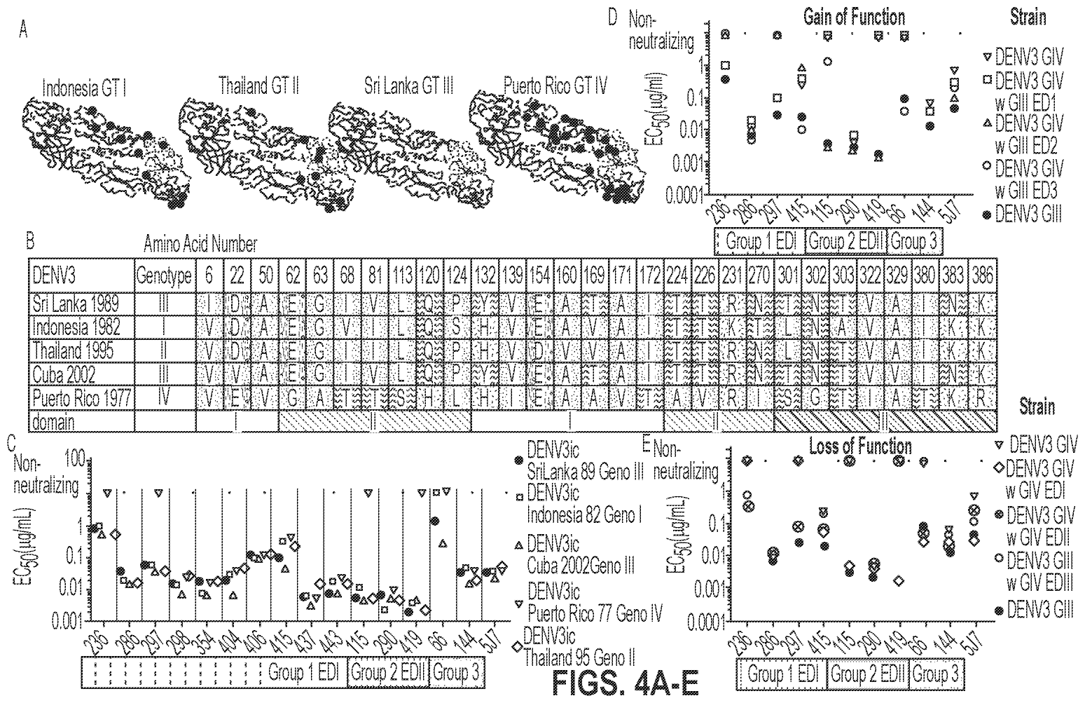

FIGS. S2A-B. Foci on Vero-81 cells. Related to FIGS. 3A-D and FIGS. 4A-E. (FIG. S2A) DENV3/1 chimeras display relatively similar foci at 48 hours on Vero-81 cells. DENV3/1 EDI/III D contains the largest transplant but grows well on Vero cells and displays slightly larger foci than the other DENV3/1 chimeric viruses. (FIG. S2B) DENV1/3 EDI chimeric viruses have relatively similar foci at 48 hours on Vero-81 cells. DENV1/3 EDI B contains the largest transplant but still grows well on Vero-81 cells.

FIG. S3. Neutralization curves for DENV1/3 chimeric viruses. Related to FIGS. 4A-E. Vero-81 FRNT neutralization curves for parental DENV3 is and chimeric DENV1/3 EDI-A and DENV1/3 EDI B are shown. Neutralization curves for DENV1/3 EDI B are very similar to neutralization curves for the parental DENV3, indicating a similar interaction of the hmAbs with DENV1/3 EDI B, whereas neutralization curves for DENV1/3 EDI A show reduced slopes.

FIG. S4. Monomer/dimer DENV3 E glycoprotein ELISA. Binding curves for individual hmAbs to ELISA plates coated with rE monomers or stabilized rE dimers. A starting concentration of 2 ng/μL of hmAb was diluted 2-fold (x-axis). Bound hmAbs were detected using anti-human-IgG-alkaline phosphatase and absorbance measurement at 405 nm. In comparison to the other group 1 antibodies, hmAbs DENV-415 and -406 did not bind detectably to recE monomers but did show weak binding to recE dimers, suggesting that they recognize a quaternary epitope. Group 1a hmAbs DENV-286, -298, -354 and -404 showed weak binding to recE monomer but higher binding to recE dimers, suggesting their epitope is presented more completely by recE dimers. Group 1b hmAbs DENV-236 and -297 showed no preference for recE dimers over monomers, implying that they may recognize functional epitopes in the EDI of a single protomer. Among the remaining members of group 1, hmAb DENV-443 showed a slight preference for binding to recE dimers at higher concentrations of antibody, while hmAb DENV-437 showed a low level of binding to dimer or monomeric recE without preference, suggesting that these mAbs may recognize unique epitope configurations. In contrast, all of the group 2 hmAbs (e.g., DENV-115, -290 and -419) showed a clear preference for binding to recE dimers, suggesting that they recognize quaternary epitopes. Group 3 hmAb DENV-66 did not neutralize a genotype II DENV3 virus, and consequently, did not bind recE monomers or dimers based on a genotype II E protein sequence, while the other group 3 hmAb DENV-144 preferred dimer recE and showed a similar pattern of binding as hmAb 5J7, which is known to bind a quaternary epitope. A recombinant form of the CR EDE1 epitope-specific hmAb C10 was used as a positive control, because its epitope is displayed on recE dimers and not monomers, as can be clearly seen in the binding pattern of rEDE1 C10.

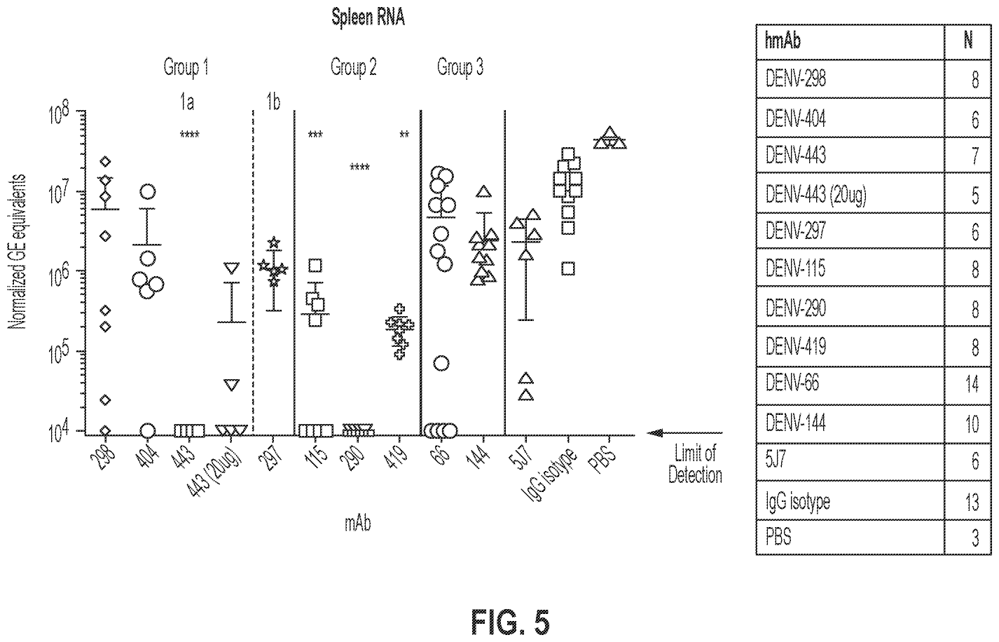

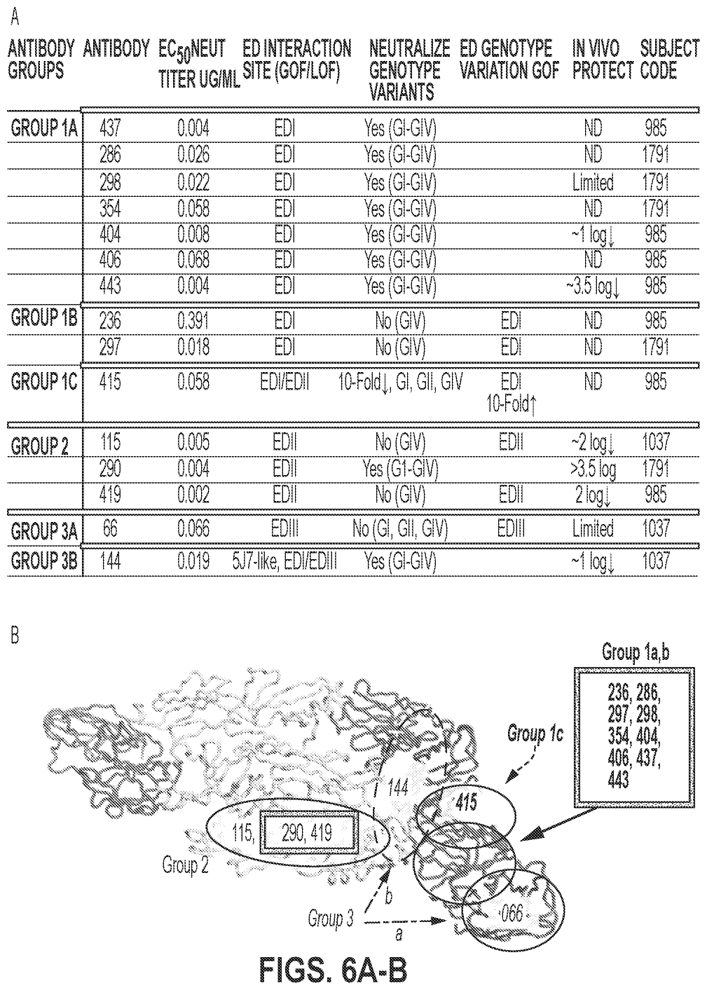

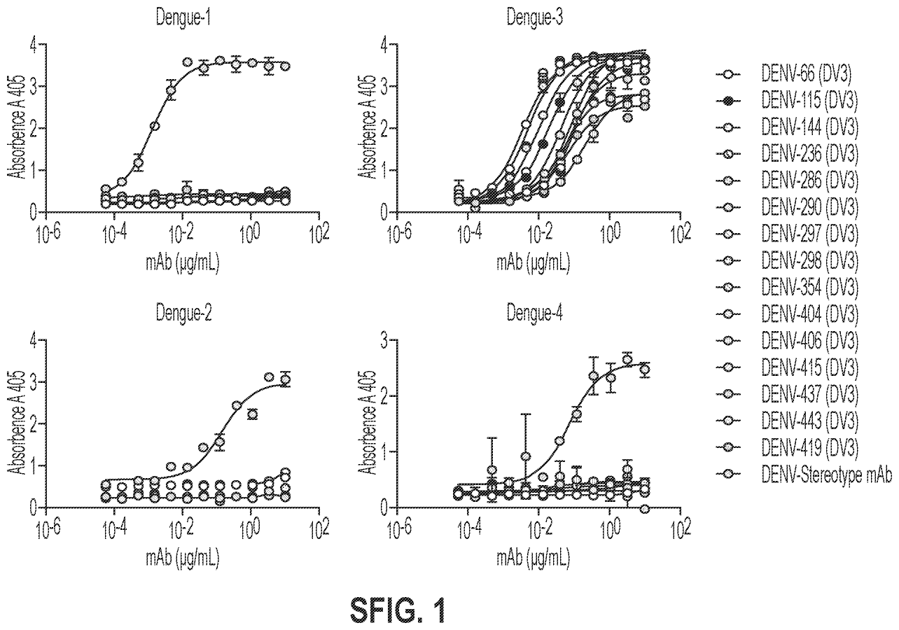

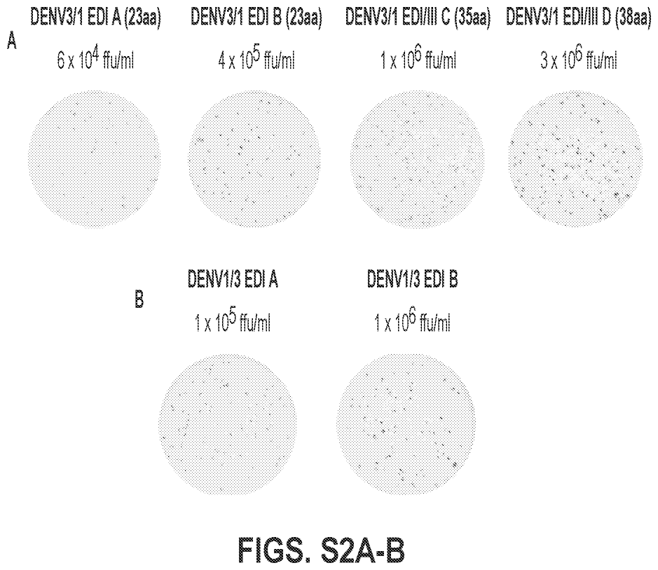

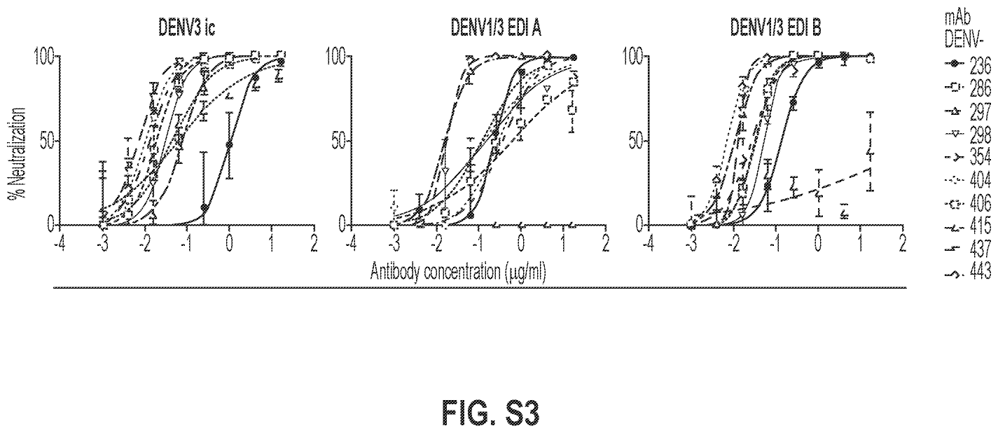

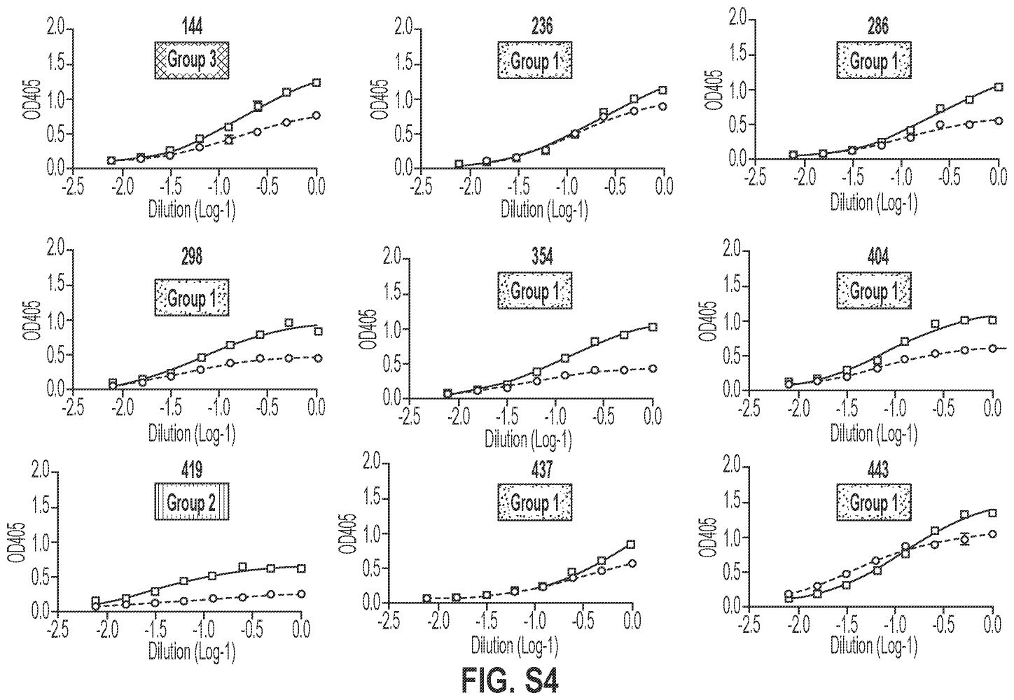

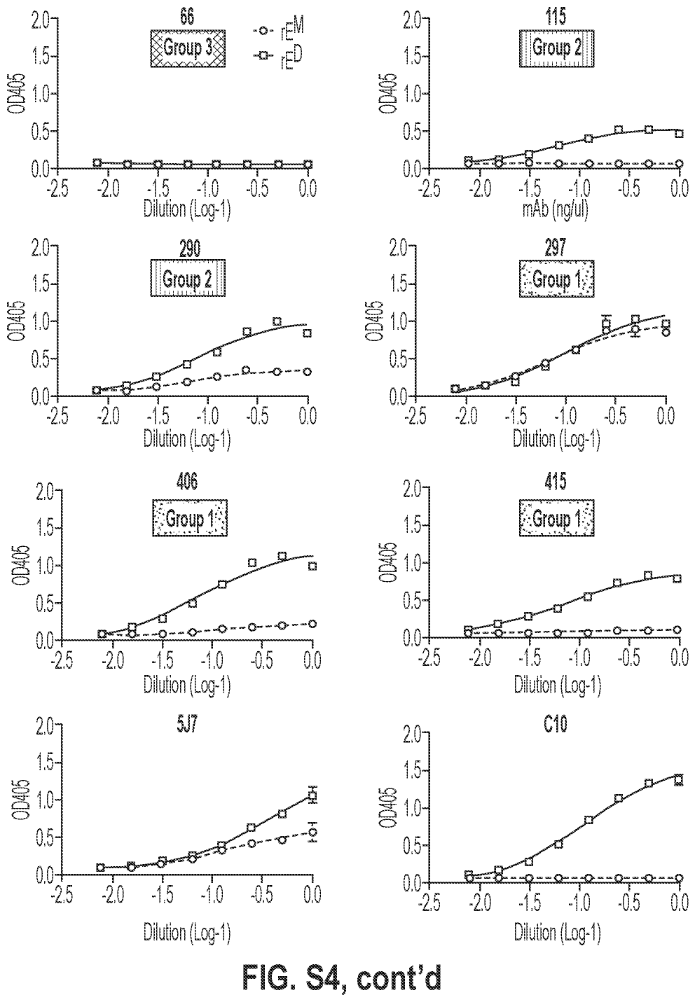

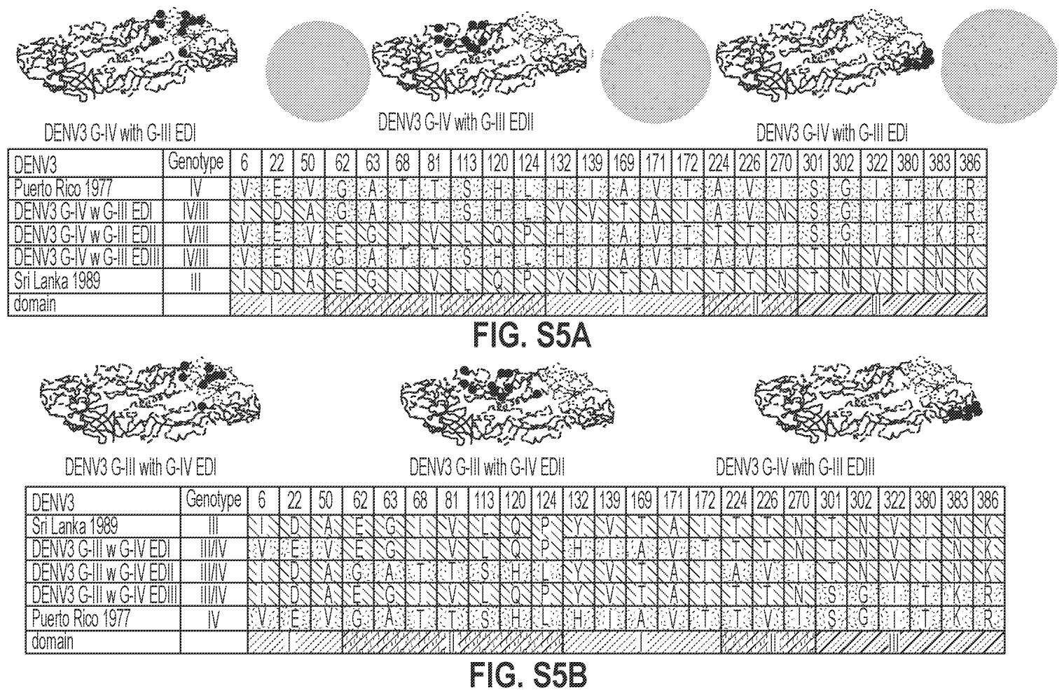

FIGS. S5A-B. Gain-of-function and loss-of-function genotype domain-swap chimeras. Related to FIG. 5 and FIGS. 6A-B. (FIG. SSA) Gain-of-Function DENV3 genotype chimeric viruses. DENV3 G-IV backbone with EDI, EDII or EDII changed to G-III. PyMOL software representations of gain-of-function viruses. Transferred residues from a DENV3 Sri Lanka strain are designated by black spheres on a DENV3 Puerto Rico strain backbone. Foci at 48 hours on Vero-81 cells are shown Amino acid alignment of changes residues is shown. Puerto Rico DENV3 residues are shown in purple and Sri Lanka DENV3 residues are shown in blue. Domain map of residues is shown in gray. (FIG. S5B) Loss-of-Function DENV3 genotype chimeric viruses. DENV3 G-III backbone with EDI, EDII or EDII changed to G-IV. PyMOL software representations of loss-of-function viruses. Transferred residues from Puerto Rico DENV3 are designated by black spheres on a Sri Lanka DENV3 backbone. Foci at 48 hours on Vero-81 cells are shown Amino acid alignment of changes residues is shown. Puerto Rico DENV3 residues are shown in purple and Sri Lanka DENV3 residues are shown in blue. Domain map of residues is shown in gray.

### DESCRIPTION OF ILLUSTRATIVE EMBODIMENTS

The natural history of flavivirus infections has been described in a prospective, pediatric cohort in Nicaragua (Andrade et al., 2017; Katzelnick et al., 2015). The availability of serum and cryopreserved peripheral blood mononuclear cell (PBMC) samples collected from subjects in this pediatric cohort provides a unique opportunity to characterize the TS immune response following DENV3 infection of children. Here, the inventor reports the isolation and characterization of fifteen TS DENV3 neutralizing hmAbs isolated from three individuals in this cohort. Using wild-type and recombinant DENV3 genotype E glycoprotein variants, coupled with detailed molecular mapping studies using epitope transplant gain- or loss-of-function recombinant viruses and other mapping techniques, six new TS antigenic sites in the DENV3 E protein that were recognized by neutralizing mAbs were identified. Finally, selected hmAbs were protective against virus replication in a prophylaxis mouse model. These results indicate that multiple antigenic sites on the DENV3 E protein are recognized by human neutralizing and protective antibodies.

These and other aspects of the disclosure are set forth in greater detail below.

### I. DENGUE VIRUS

A. General background

The dengue virus (DENV) in one of four (possibly now five) serotypes is the cause of dengue fever. It is a mosquito-borne single positive-stranded RNA virus of the family

Flaviviridae; genus Flavivirus. All five serotypes can cause the full spectrum of disease. Its genome is about 11,000 bases that codes for three structural proteins, capsid protein C, membrane protein M, envelope protein E; seven nonstructural proteins, NS1, NS2a, NS2b, NS3, NS4a, NS4b, NSS; and short non-coding regions on both the 5′ and 3′ ends. Further classification of each serotype into genotypes often relates to the region where particular strains are commonly found or were first found.

The dengue type 1 virus appears to have evolved in the early 19th century. Based on the analysis of the envelope protein there are at least four genotypes (1 to 4). The rate of nucleotide substitution for this virus has been estimated to be 6.5×10-4 per nucleotide per year, a rate similar to other RNA viruses. The American African genotype has been estimated to have evolved from 1907 to 1949. Until a few hundred years ago dengue virus was transmitted in sylvatic cycles in Africa and Asia between mosquitoes of the genus Aedes and non-human primates with rare emergences into human populations. The global spread of dengue virus, however, has followed its emergence from sylvatic cycles and the primary life cycle now exclusively involves transmission between humans and Aedes mosquitoes. Vertical transmission from mosquito to mosquito has also been observed in some vector species.

B. Structure

The DENV E (envelope) protein, found on the viral surface, is important in the initial attachment of the viral particle to the host cell. Dengue virus is transmitted by a mosquito known as Aedes. Several molecules which interact with the viral E protein (ICAM3-grabbing non-integrin, CD209, Rab 5, GRP 78, and the mannose receptor) have been shown to be important factors mediating attachment and viral entry.

The DENV prM (membrane) protein, which is important in the formation and maturation of the viral particle, consists of seven antiparallel β-strands stabilized by three disulfide bonds. The glycoprotein shell of the mature DENV virion consists of 180 copies each of the E protein and M protein. The immature virion starts out with the E and prM proteins forming 90 heterodimers that give a spiky exterior to the viral particle. This immature viral particle buds into the endoplasmic reticulum and eventually travels via the secretory pathway to the Golgi apparatus. As the virion passes through the trans-Golgi Network (TGN) it is exposed to low pH. This acidic environment causes a conformational change in the E protein which disassociates it from the prM protein and causes it to form E homodimers. These homodimers lie flat against the viral surface giving the maturing virion a smooth appearance.

During this maturation pr peptide is cleaved from the M peptide by the host protease, furin. The M protein then acts as a transmembrane protein under the E-protein shell of the mature virion. The pr peptide stays associated with the E protein until the viral particle is released into the extracellular environment. This pr peptide acts like a cap, covering the hydrophobic fusion loop of the E protein until the viral particle has exited the cell.

The DENV NS3 protein is a serine protease, as well as an RNA helicase and RTPase/NTPase. The protease domain consists of six β-strands arranged into two β-barrels formed by residues 1-180 of the protein. The catalytic triad (His-51, Asp-75 and Ser-135), is found between these two β-barrels, and its activity is dependent on the presence of the NS2B cofactor. This cofactor wraps around the NS3 protease domain and becomes part of the active site. The remaining NS3 residues (180-618), form the three subdomains of the DENV helicase. A six-stranded parallel β-sheet surrounded by four α-helices make up subdomains I and II, and subdomain III is composed of 4 α-helices surrounded by three shorter α-helices and two antiparallel β-strands.

The DENV NS5 protein is a 900-residue peptide with a methyltransferase domain at its N-terminal end (residues 1-296) and an RNA-dependent RNA polymerase (RdRp) at its C-terminal end (residues 320-900). The methyltransferase domain consists of an α/β/β sandwich flanked by N-and C-terminal subdomains. The DENV RdRp is similar to other RdRps containing palm, finger, and thumb subdomains and a GDD motif for incorporating nucleotides.

C. Severe Disease

The reason that some people suffer from more severe forms of dengue, such as dengue hemorrhagic fever, is multifactorial. Different strains of viruses interacting with people with different immune backgrounds lead to a complex interaction. Among the possible causes are cross-serotypic immune response, through a mechanism known as antibody-dependent enhancement, which happens when a person who has been previously infected with dengue gets infected for the second, third or fourth time. The previous antibodies to the old strain of dengue virus now interfere with the immune response to the current strain, leading paradoxically to more virus entry and uptake.

D. Immune System Interaction

In recent years, many studies have shown that flaviviruses, especially dengue virus has the ability to inhibit the innate immune response during the infection. Indeed, the dengue virus has many nonstructural proteins that allow the inhibition of various mediators of the innate immune system response. These proteins act on two levels:
Inhibition of interferon signaling by blocking signal transducer. NS4B it is a small hydrophobic protein located in association with the endoplasmic reticulum. It may block the phosphorylation of STAT 1 after induction by interferons type I alpha, beta. In fact, the activity of Tyk2 kinase decreases with the dengue virus, so STAT 1 phosphorylation decreases too. Therefore, the innate immune system response may be blocked. Thus, there is no production of ISG. NS2A and NS4A cofactor may also take part in the STAT 1 inhibition. The presence of the 105 kDa NS5 protein results in inactivation of STAT2 (via the signal transduction of the response to interferon) when it is expressed alone. When NS5 is cleaved with NS4B by a protease (NS2B3) it can degrade STAT2. In fact, after the cleavage of NS5 by the protease, there is an E3 ligase association with STAT2, and the E3 ligase targets STAT2 for the degradation
Inhibition of the type I interferon response. NS2B3-b protease complex is a proteolytic core consisting of the last 40 amino acids of NS2B and the first 180 amino acids of NS3. Cleavage of the NS2B3 precursor activates the protease complex. This protease complex allows the inhibition of the production of type I interferon by reducing the activity of IFN-β promoter: studies have shown that NS2B3 protease complex is involved in inhibiting the phosphorylation of IRF3. A recent study shows that the NS2B3 protease complex inhibits (by cleaving) protein MITA which allows the IRF3 activation.

E. Vaccine Research

There currently is no human vaccine available. Several vaccines are under development by private and public researchers. Developing a vaccine against the disease is challenging. With five different serotypes of the dengue virus that can cause the disease, the vaccine must immunize against all five types to be effective. Vaccination against only one serotype could possibly lead to severe dengue hemorrhagic shock (DHS) when infected with another serotype due to antibody-dependent enhancement. When infected with dengue virus, the immune system produces cross-reactive antibodies that provide immunity to that particular serotype. However, these antibodies are incapable of neutralizing any other serotypes upon reinfection and actually serve to increases viral infection. When macrophages consume the ‘neutralized’ virus, the virus is able replicate within the macrophage. In all, these cross-reactive, ineffective antibodies ease the access of these viruses into macrophages, which induces the dengue hemorrhagic fever. A common problem faced in dengue-endemic regions is when mothers become infected with dengue; after giving birth, offspring carry the immunity from their mother and are susceptible to hemorrhagic fever if infected with any of the other four serotypes. One vaccine was in phase III trials in 2012 and planning for vaccine usage and effectiveness surveillance had started. In September 2012, it was announced that one of the vaccines had not done well in clinical trials.

In 2009, Sanofi-Pasteur started building a new facility. This unit produces 4 serotypes vaccine for phase III trials. In September 2014, Sanofi-Pasteur CEO gives early results of the phase III trial efficacy study in Latin America. The efficacy per serotype (ST) varied widely, from 42.3% for ST2, rising to 50.3% for ST1, and to 74.0% for ST3 and 77.7% for ST4.

### II. MONOCLONAL ANTIBODIES AND PRODUCTION THEREOF

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In particular embodiments, the antibody is purified: (1) to greater than 95% by weight of antibody as determined by the Lowry method, and most particularly more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

The basic four-chain antibody unit is a heterotetrameric glycoprotein composed of two identical light (L) chains and two identical heavy (H) chains. An IgM antibody consists of 5 basic heterotetramer units along with an additional polypeptide called J chain, and therefore contain 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable region (V_{H}) followed by three constant domains (C_{H}) for each of the alpha and gamma chains and four C_{H} domains for mu and isotypes. Each L chain has at the N-terminus, a variable region (V_{L}) followed by a constant domain (C_{L}) at its other end. The V_{L} is aligned with the V_{H} and the C_{L} is aligned with the first constant domain of the heavy chain (C_{H1}). Particular amino acid residues are believed to form an interface between the light chain and heavy chain variable regions. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

The L chain from any vertebrate species can be assigned to one of two clearly distinct types, called kappa and lambda based on the amino acid sequences of their constant domains (C_{L}). Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated alpha, delta, epsilon, gamma and mu, respectively. They gamma and alpha classes are further divided into subclasses on the basis of relatively minor differences in C_{H} sequence and function, humans express the following subclasses: IgG1, IgG2, IgG3, IgG4, IgA1, and IgA2.

The term “variable” refers to the fact that certain segments of the V domains differ extensively in sequence among antibodies. The V domain mediates antigen binding and defines specificity of a particular antibody for its particular antigen. However, the variability is not evenly distributed across the 110-amino acid span of the variable regions. Instead, the V regions consist of relatively invariant stretches called framework regions (FRs) of 15-30 amino acids separated by shorter regions of extreme variability called “hypervariable regions” that are each 9-12 amino acids long. The variable regions of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC), antibody-dependent cellular phagocytosis (ADCP), antibody-dependent neutrophil phagocytosis (ADNP), and antibody-dependent complement deposition (ADCD).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. et al. Nucl. Acids Res. 28:219-221 (2000)). Optionally the antibody has symmetrical insertions at one or more of the following points 28, 36 (L1), 63, 74-75 (L2) and 123 (L3) in the V_{L}, and 28, 36 (H1), 63, 74-75 (H2) and 123 (H3) in the V_{sub}H when numbered in accordance with AHo; Honneger, A. and Plunkthun, A. J. Mol. Biol. 309:657-670 (2001)).

By “germline nucleic acid residue” is meant the nucleic acid residue that naturally occurs in a germline gene encoding a constant or variable region. “Germline gene” is the DNA found in a germ cell (i.e., a cell destined to become an egg or in the sperm). A “germline mutation” refers to a heritable change in a particular DNA that has occurred in a germ cell or the zygote at the single-cell stage, and when transmitted to offspring, such a mutation is incorporated in every cell of the body. A germline mutation is in contrast to a somatic mutation which is acquired in a single body cell. In some cases, nucleotides in a germline DNA sequence encoding for a variable region are mutated (i.e., a somatic mutation) and replaced with a different nucleotide.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present disclosure may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567) after single cell sorting of an antigen specific B cell, an antigen specific plasmablast responding to an infection or immunization, or capture of linked heavy and light chains from single cells in a bulk sorted antigen specific collection. The “monoclonal antibodies” may also be isolated from phage antibody libraries using the techniques described in Clackson et al., Nature, 352:624-628 (1991) and Marks et al., J. Mol. Biol., 222:581-597 (1991), for example.

A. General Methods

It will be understood that monoclonal antibodies binding to dengue virus will have several applications. These include the production of diagnostic kits for use in detecting and diagnosing dengue virus infection, as well as for treating the same. In these contexts, one may link such antibodies to diagnostic or therapeutic agents, use them as capture agents or competitors in competitive assays, or use them individually without additional agents being attached thereto. The antibodies may be mutated or modified, as discussed further below. Methods for preparing and characterizing antibodies are well known in the art (see, e.g., Antibodies: A Laboratory Manual, Cold Spring Harbor Laboratory, 1988; U.S. Pat. No. 4,196,265).

The methods for generating monoclonal antibodies (MAbs) generally begin along the same lines as those for preparing polyclonal antibodies. The first step for both these methods is immunization of an appropriate host or identification of subjects who are immune due to prior natural infection or vaccination with a licensed or experimental vaccine. As is well known in the art, a given composition for immunization may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine. As also is well known in the art, the immunogenicity of a particular immunogen composition can be enhanced by the use of non-specific stimulators of the immune response, known as adjuvants. Exemplary and preferred adjuvants in animals include complete Freund's adjuvant (a non-specific stimulator of the immune response containing killed Mycobacterium tuberculosis), incomplete Freund's adjuvants and aluminum hydroxide adjuvant and in humans include alum, CpG, MFP59 and combinations of immunostimulatory molecules (“Adjuvant Systems”, such as AS01 or AS03). Additional experimental forms of inoculation to induce Dengue-specific B cells is possible, including nanoparticle vaccines, or gene-encoded antigens delivered as DNA or RNA genes in a physical delivery system (such as lipid nanoparticle or on a gold biolistic bead), and delivered with needle, gene gun, transcutaneous electroporation device. The antigen gene also can be carried as encoded by a replication competent or defective viral vector such as adenovirus, adeno-associated virus, poxvirus, herpesvirus, or alphavirus replicon, or alternatively a virus like particle.

In the case of human antibodies against natural pathogens, a suitable approach is to identify subjects that have been exposed to the pathogens, such as those who have been diagnosed as having contracted the disease, or those who have been vaccinated to generate protective immunity against the pathogen or to test the safety or efficacy of an experimental vaccine. Circulating anti-pathogen antibodies can be detected, and antibody encoding or producing B cells from the antibody-positive subject may then be obtained.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, also may be given. The process of boosting and titering is repeated until a suitable titer is achieved. When a desired level of immunogenicity is obtained, the immunized animal can be bled and the serum isolated and stored, and/or the animal can be used to generate mAbs.

Following immunization, somatic cells with the potential for producing antibodies, specifically B lymphocytes (B cells), are selected for use in the mAb generating protocol. These cells may be obtained from biopsied spleens, lymph nodes, tonsils or adenoids, bone marrow aspirates or biopsies, tissue biopsies from mucosal organs like lung or GI tract, or from circulating blood. The antibody-producing B lymphocytes from the immunized animal or immune human are then fused with cells of an immortal myeloma cell, generally one of the same species as the animal that was immunized or human or human/mouse chimeric cells. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas). Any one of a number of myeloma cells may be used, as are known to those of skill in the art (Goding, pp. 65-66, 1986; Campbell, pp. 75-83, 1984). HMMA2.5 cells or MFP-2 cells are particularly useful examples of such cells.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. In some cases, transformation of human B cells with Epstein Barr virus (EBV) as an initial step increases the size of the B cells, enhancing fusion with the relatively large-sized myeloma cells. Transformation efficiency by EBV is enhanced by using CpG and a Chk2 inhibitor drug in the transforming medium. Alternatively, human B cells can be activated by co-culture with transfected cell lines expressing CD40 Ligand (CD154) in medium containing additional soluble factors, such as IL-21 and human B cell Activating Factor (BAFF), a Type II member of the TNF superfamily Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding, pp. 71-74, 1986) and there are processes for better efficiency (Yu et al., 2008). Fusion procedures usually produce viable hybrids at low frequencies, about 1×10⁻⁶ to 1×10⁻⁸, but with optimized procedures one can achieve fusion efficiencies close to 1 in 200 (Yu et al., 2008). However, relatively low efficiency of fusion does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture medium. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the medium is supplemented with hypoxanthine and thymidine as a source of nucleotides (HAT medium). Where azaserine is used, the medium is supplemented with hypoxanthine. Ouabain is added if the B cell source is an EBV-transformed human B cell line, in order to eliminate EBV-transformed lines that have not fused to the myeloma.

The preferred selection medium is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain may also be used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like. The selected hybridomas are then serially diluted or single-cell sorted by flow cytometric sorting and cloned into individual antibody-producing cell lines, which clones can then be propagated indefinitely to provide mAbs. The cell lines may be exploited for mAb production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide mAbs in high concentration. The individual cell lines could also be cultured in vitro, where the mAbs are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

MAbs produced by either means may be further purified, if desired, using filtration, centrifugation and various chromatographic methods such as FPLC or affinity chromatography. Fragments of the monoclonal antibodies of the disclosure can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

It also is contemplated that a molecular cloning approach may be used to generate monoclonal antibodies. Single B cells labelled with the antigen of interest can be sorted physically using paramagnetic bead selection or flow cytometric sorting, then RNA can be isolated from the single cells and antibody genes amplified by RT-PCR. Alternatively, antigen-specific bulk sorted populations of cells can be segregated into microvesicles and the matched heavy and light chain variable genes recovered from single cells using physical linkage of heavy and light chain amplicons, or common barcoding of heavy and light chain genes from a vesicle. Matched heavy and light chain genes form single cells also can be obtained from populations of antigen specific B cells by treating cells with cell-penetrating nanoparticles bearing RT-PCR primers and barcodes for marking transcripts with one barcode per cell. The antibody variable genes also can be isolated by RNA extraction of a hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 10⁴ times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present disclosure include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

B. Antibodies of the Present Disclosure

Antibodies according to the present disclosure may be defined, in the first instance, by their binding specificity. Those of skill in the art, by assessing the binding specificity/affinity of a given antibody using techniques well known to those of skill in the art, can determine whether such antibodies fall within the scope of the instant claims. For example, the epitope to which a given antibody bind may consist of a single contiguous sequence of 3 or more (e.g., 3, 4, 5, 6, 7, 8, 9, 10, 11,12, 13, 14, 15, 16, 17, 18, 19, 20) amino acids located within the antigen molecule (e.g., a linear epitope in a domain). Alternatively, the epitope may consist of a plurality of non-contiguous amino acids (or amino acid sequences) located within the antigen molecule (e.g., a conformational epitope).

Various techniques known to persons of ordinary skill in the art can be used to determine whether an antibody “interacts with one or more amino acids” within a polypeptide or protein. Exemplary techniques include, for example, routine cross-blocking assays, such as that described in Antibodies, Harlow and Lane (Cold Spring Harbor Press, Cold Spring Harbor, N.Y.). Cross-blocking can be measured in various binding assays such as ELISA, biolayer interferometry, or surface plasmon resonance. Other methods include alanine scanning mutational analysis, peptide blot analysis (Reineke (2004) Methods Mol. Biol. 248: 443-63), peptide cleavage analysis, high-resolution electron microscopy techniques using single particle reconstruction, cryoEM, or tomography, crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267: 252-259; Engen and Smith (2001) Anal. Chem. 73: 256A-265A. When the antibody neutralizes dengue virus, antibody escape mutant variant organisms can be isolated by propagating dengue virus in vitro or in animal models in the presence of high concentrations of the antibody. Sequence analysis of the dengue virus gene encoding the antigen targeted by the antibody reveals the mutation(s) conferring antibody escape, indicating residues in the epitope or that affect the structure of the epitope allosterically.

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies Profiling directed against the same antigen according to the similarities of the binding profile of each antibody to chemically or enzymatically modified antigen surfaces (see US 2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce mAbs having the desired characteristics. MAP may be used to sort the antibodies of the disclosure into groups of antibodies binding different epitopes.

The present disclosure includes antibodies that may bind to the same epitope, or a portion of the epitope. Likewise, the present disclosure also includes antibodies that compete for binding to a target or a fragment thereof with any of the specific exemplary antibodies described herein. One can easily determine whether an antibody binds to the same epitope as, or competes for binding with, a reference antibody by using routine methods known in the art. For example, to determine if a test antibody binds to the same epitope as a reference, the reference antibody is allowed to bind to target wader saturating conditions. Next, the ability of a test antibody to bind to the target molecule is assessed. If the test antibody is able to bind to the target molecule following saturation binding with the reference antibody, it can be concluded that the test antibody binds to a different epitope than the reference antibody. On the other hand, if the test antibody is not able to bind to the target molecule following saturation binding with the reference antibody, then the test antibody may bind to the same epitope as the epitope bound by the reference antibody.

To determine if an antibody competes for binding with a reference anti-dengue virus antibody, the above-described binding methodology is performed in two orientations: In a first orientation, the reference antibody is allowed to bind to the dengue virus antigen under saturating conditions followed by assessment of binding of the test antibody to the dengue virus molecule. In a second orientation, the test antibody is allowed to bind to the dengue virus antigen molecule under saturating conditions followed by assessment of binding of the reference antibody to the dengue virus molecule. If, in both orientations, only the first (saturating) antibody is capable of binding to the dengue virus, then it is concluded that the test antibody and the reference antibody compete for binding to the dengue virus. As will be appreciated by a person of ordinary skill in the art, an antibody that competes for binding with a reference antibody may not necessarily bind to the identical epitope as the reference antibody but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res, 1990 50:1495-1502), Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art. Structural studies with EM or crystallography also can demonstrate whether or not two antibodies that compete for binding recognize the same epitope.

In another aspect, there are provided monoclonal antibodies having clone-paired CDRs from the heavy and light chains as illustrated in Tables 3 and 4, respectively. Such antibodies may be produced by the clones discussed below in the Examples section using methods described herein.

In another aspect, the antibodies may be defined by their variable sequence, which include additional “framework” regions. These are provided in Tables 1 and 2 that encode or represent full variable regions. Furthermore, the antibodies sequences may vary from these sequences, optionally using methods discussed in greater detail below. For example, nucleic acid sequences may vary from those set out above in that (a) the variable regions may be segregated away from the constant domains of the light and heavy chains, (b) the nucleic acids may vary from those set out above while not affecting the residues encoded thereby, (c) the nucleic acids may vary from those set out above by a given percentage, e.g., 70%, 75%, 80%, 85%, 90%, 91%, 92%, 93%, 94%, 95%, 96%, 97%, 98% or 99% homology, (d) the nucleic acids may vary from those set out above by virtue of the ability to hybridize under high stringency conditions, as exemplified by low salt and/or high temperature conditions, such as provided by about 0.02 M to about 0.15 M NaCl at temperatures of about 50° C. to about 70° C., (e) the amino acids may vary from those set out above by a given percentage, e.g., 80%, 85%, 90%, 91%, 92%, 93%, 94%, 95%, 96%, 97%, 98% or 99% homology, or (f) the amino acids may vary from those set out above by permitting conservative substitutions (discussed below). Each of the foregoing applies to the nucleic acid sequences set forth as Table 1 and the amino acid sequences of Table 2.

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogeny pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One particular example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the disclosure. Software for performing BLAST analyses is publicly available through the National Center for Biotechnology Information. The rearranged nature of an antibody sequence and the variable length of each gene requires multiple rounds of BLAST searches for a single antibody sequence. Also, manual assembly of different genes is difficult and error-prone. The sequence analysis tool IgBLAST (world-wide-web at ncbi.nlm.nih.gov/igblast/) identifies matches to the germline V, D and J genes, details at rearrangement junctions, the delineation of Ig V domain framework regions and complementarity determining regions. IgBLAST can analyze nucleotide or protein sequences and can process sequences in batches and allows searches against the germline gene databases and other sequence databases simultaneously to minimize the chance of missing possibly the best matching germline V gene.

In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

Yet another way of defining an antibody is as a “derivative” of any of the below-described antibodies and their antigen-binding fragments. The term “derivative” refers to an antibody or antigen-binding fragment thereof that specifically binds to an antigen but which comprises, one, two, three, four, five or more amino acid substitutions, additions, deletions or modifications relative to a “parental” (or wild-type) molecule. Such amino acid substitutions or additions may introduce naturally occurring (i.e., DNA-encoded) or non-naturally occurring amino acid residues. The term “derivative” encompasses, for example, as variants having altered CHL hinge, CH2, CH3 or CH4 regions, so as to form, for example antibodies, etc., having variant Fc regions that exhibit enhanced or impaired effector or binding characteristics. The term “derivative” additionally encompasses non-amino acid modifications, for example, amino acids that may be glycosylated (e.g., have altered mannose, 2-N-acetylglucosamine, galactose, fucose, glucose, sialic acid, S-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc. In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment, the altered carbohydrate modifications enhance antibody mediated effector function relative to the antibody lacking the carbohydrate modification. Carbohydrate modifications that lead to altered antibody mediated effector function are well known in the art (for example, see Shields, R. L. et al. (2002), J. Biol. Chem. 277(30): 26733-26740; Davies J. et al. (2001), Biotechnology & Bioengineering 74(4): 288-294). Methods of altering carbohydrate contents are known to those skilled in the art, see, e.g., Wallick, S. C. et al. (1988), J. Exp. Med. 168(3): 1099-1109; Tao, M. H. et al. (1989), J. Immunol. 143(8): 2595-2601; Routledge, E. G. et al. (1995), Transplantation 60(8):847-53; Elliott, S. et al. (2003), Nature Biotechnol. 21:414-21; Shields, R. L. et al. (2002), J. Biol. Chem. 277(30): 26733-26740).

A derivative antibody or antibody fragment can be generated with an engineered sequence or glycosylation state to confer preferred levels of activity in antibody dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

A derivative antibody or antibody fragment may be modified by chemical modifications using techniques known to those of skill in the art, including, but not limited to, specific chemical cleavage, acetylation, formulation, metabolic synthesis of tunicamycin, etc. In one embodiment, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody.

C. Engineering of Antibody Sequences

In various embodiments, one may choose to engineer sequences of the identified antibodies for a variety of reasons, such as improved expression, improved cross-reactivity or diminished off-target binding. Modified antibodies may be made by any technique known to those of skill in the art, including expression through standard molecular biological techniques, or the chemical synthesis of polypeptides. Methods for recombinant expression are addressed elsewhere in this document. The following is a general discussion of relevant goals techniques for antibody engineering.

Hybridomas may be cultured, then cells lysed, and total RNA extracted. Random hexamers may be used with RT to generate cDNA copies of RNA, and then PCR performed using a multiplex mixture of PCR primers expected to amplify all human variable gene sequences. PCR product can be cloned into pGEM-T Easy vector, then sequenced by automated DNA sequencing using standard vector primers. Assay of binding and neutralization may be performed using antibodies collected from hybridoma supernatants and purified by FPLC, using Protein G columns.

Recombinant full-length IgG antibodies can be generated by subcloning heavy and light chain Fv DNAs from the cloning vector into an IgG plasmid vector, transfected into 293 (e.g., Freestyle) cells or CHO cells, and antibodies can be collected and purified from the 293 or CHO cell supernatant. Other appropriate host cells systems include bacteria, such as E. coli, insect cells (S2, Sf9, Sf29, High Five), plant cells (e.g., tobacco, with or without engineering for human-like glycans), algae, or in a variety of non-human transgenic contexts, such as mice, rats, goats or cows.

Expression of nucleic acids encoding antibodies, both for the purpose of subsequent antibody purification, and for immunization of a host, is also contemplated. Antibody coding sequences can be RNA, such as native RNA or modified RNA. Modified RNA contemplates certain chemical modifications that confer increased stability and low immunogenicity to mRNAs, thereby facilitating expression of therapeutically important proteins. For instance, N1-methyl-pseudouridine (N1mΨ) outperforms several other nucleoside modifications and their combinations in terms of translation capacity. In addition to turning off the immune/eIF2α phosphorylation-dependent inhibition of translation, incorporated N1mΨ nucleotides dramatically alter the dynamics of the translation process by increasing ribosome pausing and density on the mRNA. Increased ribosome loading of modified mRNAs renders them more permissive for initiation by favoring either ribosome recycling on the same mRNA or de novo ribosome recruitment. Such modifications could be used to enhance antibody expression in vivo following inoculation with RNA. The RNA, whether native or modified, may be delivered as naked RNA or in a delivery vehicle, such as a lipid nanoparticle.

Alternatively, DNA encoding the antibody may be employed for the same purposes. The DNA is included in an expression cassette comprising a promoter active in the host cell for which it is designed. The expression cassette is advantageously included in a replicable vector, such as a conventional plasmid or minivector. Vectors include viral vectors, such as poxviruses, adenoviruses, herpesviruses, adeno-associated viruses, and lentiviruses are contemplated. Replicons encoding antibody genes such as alphavirus replicons based on VEE virus or Sindbis virus are also contemplated. Delivery of such vectors can be performed by needle through intramuscular, subcutaneous, or intradermal routes, or by transcutaneous electroporation when in vivo expression is desired.

The rapid availability of antibody produced in the same host cell and cell culture process as the final cGMP manufacturing process has the potential to reduce the duration of process development programs. Lonza has developed a generic method using pooled transfectants grown in CDACF medium, for the rapid production of small quantities (up to 50 g) of antibodies in CHO cells. Although slightly slower than a true transient system, the advantages include a higher product concentration and use of the same host and process as the production cell line. Example of growth and productivity of GS-CHO pools, expressing a model antibody, in a disposable bioreactor: in a disposable bag bioreactor culture (5 L working volume) operated in fed-batch mode, a harvest antibody concentration of 2 g/L was achieved within 9 weeks of transfection.

Antibody molecules will comprise fragments (such as F(ab′), F(ab′)₂) that are produced, for example, by the proteolytic cleavage of the mAbs, or single-chain immunoglobulins producible, for example, via recombinant means. F(ab′) antibody derivatives are monovalent, while F(ab′)₂ antibody derivatives are bivalent. In one embodiment, such fragments can be combined with one another, or with other antibody fragments or receptor ligands to form “chimeric” binding molecules. Significantly, such chimeric molecules may contain substituents capable of binding to different epitopes of the same molecule.

In related embodiments, the antibody is a derivative of the disclosed antibodies, e.g., an antibody comprising the CDR sequences identical to those in the disclosed antibodies (e.g., a chimeric, or CDR-grafted antibody). Alternatively, one may wish to make modifications, such as introducing conservative changes into an antibody molecule. In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

It is understood that an amino acid can be substituted for another having a similar hydrophilicity and produce a biologically or immunologically modified protein. In such changes, the substitution of amino acids whose hydrophilicity values are within±2 is preferred, those that are within±1 are particularly preferred, and those within±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present disclosure also contemplates isotype modification. By modifying the Fc region to have a different isotype, different functionalities can be achieved. For example, changing to IgG₁ can increase antibody dependent cell cytotoxicity, switching to class A can improve tissue distribution, and switching to class M can improve valency.

Alternatively or additionally, it may be useful to combine amino acid modifications with one or more further amino acid modifications that alter C1q binding and/or the complement dependent cytotoxicity (CDC) function of the Fc region of an IL-23p19 binding molecule. The binding polypeptide of particular interest may be one that binds to C1q and displays complement dependent cytotoxicity. Polypeptides with pre-existing C1q binding activity, optionally further having the ability to mediate CDC may be modified such that one or both of these activities are enhanced Amino acid modifications that alter C1q and/or modify its complement dependent cytotoxicity function are described, for example, in WO/0042072, which is hereby incorporated by reference.

One can design an Fc region of an antibody with altered effector function, e.g., by modifying C1q binding and/or FcγR binding and thereby changing CDC activity and/or ADCC activity. “Effector functions” are responsible for activating or diminishing a biological activity (e.g., in a subject). Examples of effector functions include, but are not limited to: C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of an antibody with improved C1q binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

FcRn binding. Fc mutations can also be introduced and engineered to alter their interaction with the neonatal Fc receptor (FcRn) and improve their pharmacokinetic properties. A collection of human Fc variants with improved binding to the FcRn have been described (Shields et al., (2001). High resolution mapping of the binding site on human IgG1 for FcγRI, FcγRII, FcγRIII, and FcRn and design of IgG1 variants with improved binding to the FcγR, (J. Biol. Chem. 276:6591-6604). A number of methods are known that can result in increased half-life (Kuo and Aveson, (2011)), including amino acid modifications may be generated through techniques including alanine scanning mutagenesis, random mutagenesis and screening to assess the binding to the neonatal Fc receptor (FcRn) and/or the in vivo behavior. Computational strategies followed by mutagenesis may also be used to select one of amino acid mutations to mutate.

The present disclosure therefore provides a variant of an antigen binding protein with optimized binding to FcRn. In a particular embodiment, the said variant of an antigen binding protein comprises at least one amino acid modification in the Fc region of said antigen binding protein, wherein said modification is selected from the group consisting of 226, 227, 228, 230, 231, 233, 234, 239, 241, 243, 246, 250, 252, 256, 259, 264, 265, 267, 269, 270, 276, 284, 285, 288, 289, 290, 291, 292, 294, 297, 298, 299, 301, 302, 303, 305, 307, 308, 309, 311, 315, 317, 320, 322, 325, 327, 330, 332, 334, 335, 338, 340, 342, 343, 345, 347, 350, 352, 354, 355, 356, 359, 360, 361, 362, 369, 370, 371, 375, 378, 380, 382, 384, 385, 386, 387, 389, 390, 392, 393, 394, 395, 396, 397, 398, 399, 400, 401 403, 404, 408, 411, 412, 414, 415, 416, 418, 419, 420, 421, 422, 424, 426, 428, 433, 434, 438, 439, 440, 443, 444, 445, 446 and 447 of the Fc region as compared to said parent polypeptide, wherein the numbering of the amino acids in the Fc region is that of the EU index in Kabat. In a further aspect of the disclosure the modifications are M252Y/S254T/T256E.

Additionally, various publications describe methods for obtaining physiologically active molecules whose half-lives are modified, see for example Kontermann (2009) either by introducing an FcRn-binding polypeptide into the molecules or by fusing the molecules with antibodies whose FcRn-binding affinities are preserved but affinities for other Fc receptors have been greatly reduced or fusing with FcRn binding domains of antibodies.

Derivatized antibodies may be used to alter the half-lives (e.g., serum half-lives) of parental antibodies in a mammal, particularly a human Such alterations may result in a half-life of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the antibodies of the present disclosure or fragments thereof in a mammal, preferably a human, results in a higher serum titer of said antibodies or antibody fragments in the mammal, and thus reduces the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fc domain and the FcRn receptor.

Beltramello et al. (2010) previously reported the modification of neutralizing mAbs, due to their tendency to enhance dengue virus infection, by generating in which leucine residues at positions 1.3 and 1.2 of CH2 domain (according to the IMGT unique numbering for C-domain) were substituted with alanine residues. This modification, also known as “LALA” mutation, abolishes antibody binding to FcγRI, FcγRII and FcγRIIIa, as described by Hessell et al. (2007). The variant and unmodified recombinant mAbs were compared for their capacity to neutralize and enhance infection by the four dengue virus serotypes. LALA variants retained the same neutralizing activity as unmodified mAb but were completely devoid of enhancing activity. LALA mutations of this nature are therefore contemplated in the context of the presently disclosed antibodies.The subsequently developed LALA PG variant may exhibit even further reduction of FcγR-mediated activity.

Altered glycosylation. A particular embodiment of the present disclosure is an isolated monoclonal antibody, or antigen binding fragment thereof, containing a substantially homogeneous glycan without sialic acid, galactose, or fucose. The monoclonal antibody comprises a heavy chain variable region and a light chain variable region, both of which may be attached to heavy chain or light chain constant regions respectively. The aforementioned substantially homogeneous glycan may be covalently attached to the heavy chain constant region.

Another embodiment of the present disclosure comprises a mAb with a novel Fc glycosylation pattern. The isolated monoclonal antibody, or antigen binding fragment thereof, is present in a substantially homogenous composition represented by the GNGN or G1/G2 glycoform. Fc glycosylation plays a significant role in anti-viral and anti-cancer properties of therapeutic mAbs. The disclosure is in line with a recent study that shows increased anti-lentivirus cell-mediated viral inhibition of a fucose free anti-HIV mAb in vitro. This embodiment of the present disclosure with homogenous glycans lacking a core fucose, showed increased protection against specific viruses by a factor greater than two-fold. Elimination of core fucose dramatically improves the ADCC activity of mAbs mediated by natural killer (NK) cells but appears to have the opposite effect on the ADCC activity of polymorphonuclear cells (PMNs).

The isolated monoclonal antibody, or antigen binding fragment thereof, comprising a substantially homogenous composition represented by the GNGN or G1/G2 glycoform exhibits increased binding affinity for Fc gamma RI and Fc gamma RIII compared to the same antibody without the substantially homogeneous GNGN glycoform and with G0, G1F, G2F, GNF, GNGNF or GNGNFX containing glycoforms. In one embodiment of the present disclosure, the antibody dissociates from Fc gamma RI with a Kd of 1×10⁻⁸ M or less and from Fc gamma RIII with a Kd of 1×10⁻⁷ M or less.

Glycosylation of an Fc region is typically either N-linked or O-linked. N-linked refers to the attachment of the carbohydrate moiety to the side chain of an asparagine residue. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide creates a potential glycosylation site.

The glycosylation pattern may be altered, for example, by deleting one or more glycosylation site(s) found in the polypeptide, and/or adding one or more glycosylation site(s) that are not present in the polypeptide. Addition of glycosylation sites to the Fc region of an antibody is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration may also be made by the addition of, or substitution by, one or more serine or threonine residues to the sequence of the original polypeptide (for 0-linked glycosylation sites). Additionally, a change of Asn 297 to Ala can remove one of the glycosylation sites.

In certain embodiments, the antibody is expressed in cells that express beta (1,4)-N-acetylglucosaminyltransferase III (GnT III), such that GnT III adds GlcNAc to the IL-23p19 antibody. Methods for producing antibodies in such a fashion are provided in WO/9954342, WO/03011878, patent publication 20030003097A1, and Umana et al., Nature Biotechnology, 17:176-180, February 1999. Cell lines can be altered to enhance or reduce or eliminate certain post-translational modifications, such as glycosylation, using genome editing technology such as Clustered Regularly Interspaced Short Palindromic Repeats (CRISPR). For example, CRISPR technology can be used to eliminate genes encoding glycosylating enzymes in 293 or CHO cells used to express recombinant monoclonal antibodies.

Elimination of monoclonal antibody protein sequence liabilities. It is possible to engineer the antibody variable gene sequences obtained from human B cells to enhance their manufacturability and safety. Potential protein sequence liabilities can be identified by searching for sequence motifs associated with sites containing:

1) Unpaired Cys residues,

2) N-linked glycosylation,

3) Asn deamidation,

4) Asp isomerization,

5) SYE truncation,

6) Met oxidation,

7) Trp oxidation,

8) N-terminal glutamate,

9) Integrin binding,

10) CD11c/CD18 binding, or

11) Fragmentation

Such motifs can be eliminated by altering the synthetic gene for the cDNA encoding recombinant antibodies.

Protein engineering efforts in the field of development of therapeutic antibodies clearly reveal that certain sequences or residues are associated with solubility differences (Fernandez-Escamilla et al., Nature Biotech., 22 (10), 1302-1306, 2004; Chennamsetty et al., PNAS, 106 (29), 11937-11942, 2009; Voynov et al., Biocon. Chem., 21(2), 385-392, 2010) Evidence from solubility-altering mutations in the literature indicate that some hydrophilic residues such as aspartic acid, glutamic acid, and serine contribute significantly more favorably to protein solubility than other hydrophilic residues, such as asparagine, glutamine, threonine, lysine, and arginine.

Stability. Antibodies can be engineered for enhanced biophysical properties. One can use elevated temperature to unfold antibodies to determine relative stability, using average apparent melting temperatures. Differential Scanning Calorimetry (DSC) measures the heat capacity, Cₚ, of a molecule (the heat required to warm it, per degree) as a function of temperature. One can use DSC to study the thermal stability of antibodies. DSC data for mAbs is particularly interesting because it sometimes resolves the unfolding of individual domains within the mAb structure, producing up to three peaks in the thermogram (from unfolding of the Fab, C_{H}2, and C_{H}3 domains). Typically unfolding of the Fab domain produces the strongest peak. The DSC profiles and relative stability of the Fc portion show characteristic differences for the human IgG₁, IgG₂, IgG₃, and IgG₄ subclasses (Garber and Demarest, Biochem. Biophys. Res. Commun. 355, 751-757, 2007). One also can determine average apparent melting temperature using circular dichroism (CD), performed with a CD spectrometer. Far-UV CD spectra will be measured for antibodies in the range of 200 to 260 nm at increments of 0.5 nm. The final spectra can be determined as averages of 20 accumulations. Residue ellipticity values can be calculated after background subtraction. Thermal unfolding of antibodies (0.1 mg/mL) can be monitored at 235 nm from 25-95° C. and a heating rate of 1° C/min One can use dynamic light scattering (DLS) to assess for propensity for aggregation. DLS is used to characterize size of various particles including proteins. If the system is not disperse in size, the mean effective diameter of the particles can be determined. This measurement depends on the size of the particle core, the size of surface structures, and particle concentration. Since DLS essentially measures fluctuations in scattered light intensity due to particles, the diffusion coefficient of the particles can be determined. DLS software in commercial DLA instruments displays the particle population at different diameters. Stability studies can be done conveniently using DLS. DLS measurements of a sample can show whether the particles aggregate over time or with temperature variation by determining whether the hydrodynamic radius of the particle increases. If particles aggregate, one can see a larger population of particles with a larger radius. Stability depending on temperature can be analyzed by controlling the temperature in situ. Capillary electrophoresis (CE) techniques include proven methodologies for determining features of antibody stability. One can use an iCE approach to resolve antibody protein charge variants due to deamidation, C-terminal lysines, sialylation, oxidation, glycosylation, and any other change to the protein that can result in a change in pI of the protein. Each of the expressed antibody proteins can be evaluated by high throughput, free solution isoelectric focusing (IEF) in a capillary column (cIEF), using a Protein Simple Maurice instrument. Whole-column UV absorption detection can be performed every 30 seconds for real time monitoring of molecules focusing at the isoelectric points (pIs). This approach combines the high resolution of traditional gel IEF with the advantages of quantitation and automation found in column-based separations while eliminating the need for a mobilization step. The technique yields reproducible, quantitative analysis of identity, purity, and heterogeneity profiles for the expressed antibodies. The results identify charge heterogeneity and molecular sizing on the antibodies, with both absorbance and native fluorescence detection modes and with sensitivity of detection down to 0.7 μg/mL.

Solubility. One can determine the intrinsic solubility score of antibody sequences. The intrinsic solubility scores can be calculated using CamSol Intrinsic (Sormanni et al., J Mol Biol 427, 478-490, 2015). The amino acid sequences for residues 95-102 (Kabat numbering) in HCDR3 of each antibody fragment such as a scFv can be evaluated via the online program to calculate the solubility scores. One also can determine solubility using laboratory techniques. Various techniques exist, including addition of lyophilized protein to a solution until the solution becomes saturated and the solubility limit is reached, or concentration by ultrafiltration in a microconcentrator with a suitable molecular weight cut-off. The most straightforward method is induction of amorphous precipitation, which measures protein solubility using a method involving protein precipitation using ammonium sulfate (Trevino et al., J Mol Biol, 366: 449-460, 2007). Ammonium sulfate precipitation gives quick and accurate information on relative solubility values. Ammonium sulfate precipitation produces precipitated solutions with well-defined aqueous and solid phases and requires relatively small amounts of protein. Solubility measurements performed using induction of amorphous precipitation by ammonium sulfate also can be done easily at different pH values. Protein solubility is highly pH dependent, and pH is considered the most important extrinsic factor that affects solubility.

Autoreactivity. Generally, it is thought that autoreactive clones should be eliminated during ontogeny by negative selection, however it has become clear that many human naturally occurring antibodies with autoreactive properties persist in adult mature repertoires, and the autoreactivity may enhance the antiviral function of many antibodies to pathogens. It has been noted that HCDR3 loops in antibodies during early B cell development are often rich in positive charge and exhibit autoreactive patterns (Wardemann et al., Science 301, 1374-1377, 2003). One can test a given antibody for autoreactivity by assessing the level of binding to human origin cells in microscopy (using adherent HeLa or HEp-2 epithelial cells) and flow cytometric cell surface staining (using suspension Jurkat T cells and 293S human embryonic kidney cells). Autoreactivity also can be surveyed using assessment of binding to tissues in tissue arrays.

Preferred residues (“Human Likeness”). B cell repertoire deep sequencing of human B cells from blood donors is being performed on a wide scale in many recent studies. Sequence information about a significant portion of the human antibody repertoire facilitates statistical assessment of antibody sequence features common in healthy humans. With knowledge about the antibody sequence features in a human recombined antibody variable gene reference database, the position specific degree of “Human Likeness” (HL) of an antibody sequence can be estimated. HL has been shown to be useful for the development of antibodies in clinical use, like therapeutic antibodies or antibodies as vaccines. The goal is to increase the human likeness of antibodies to reduce potential adverse effects and anti-antibody immune responses that will lead to significantly decreased efficacy of the antibody drug or can induce serious health implications. One can assess antibody characteristics of the combined antibody repertoire of three healthy human blood donors of about 400 million sequences in total and created a novel “relative Human Likeness” (rHL) score that focuses on the hypervariable region of the antibody. The rHL score allows one to easily distinguish between human (positive score) and non-human sequences (negative score). Antibodies can be engineered to eliminate residues that are not common in human repertoires.

D. Single Chain Antibodies

A single chain variable fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen binding domain as a single peptide. Alternatively, scFv can be created directly from subcloned heavy and light chains derived from a hybridoma or B cell. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alanine, serine and glycine. However, other residues can function as well. Tang et al. (1996) used phage display as a means of rapidly selecting tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10⁶ different members) was displayed on filamentous phage and subjected to affinity selection with hapten. The population of selected variants exhibited significant increases in binding activity but retained considerable sequence diversity. Screening 1054 individual variants subsequently yielded a catalytically active scFv that was produced efficiently in soluble form. Sequence analysis revealed a conserved proline in the linker two residues after the V_{H} C terminus and an abundance of arginines and prolines at other positions as the only common features of the selected tethers.

The recombinant antibodies of the present disclosure may also involve sequences or moieties that permit dimerization or multimerization of the receptors. Such sequences include those derived from IgA, which permit formation of multimers in conjunction with the J-chain. Another multimerization domain is the Gal4 dimerization domain. In other embodiments, the chains may be modified with agents such as biotin/avidin, which permit the combination of two antibodies.

In a separate embodiment, a single-chain antibody can be created by joining receptor light and heavy chains using a non-peptide linker or chemical unit. Generally, the light and heavy chains will be produced in distinct cells, purified, and subsequently linked together in an appropriate fashion (i.e., the N-terminus of the heavy chain being attached to the C-terminus of the light chain via an appropriate chemical bridge).

Cross-linking reagents are used to form molecular bridges that tie functional groups of two different molecules, e.g., a stabilizing and coagulating agent. However, it is contemplated that dimers or multimers of the same analog or heteromeric complexes comprised of different analogs can be created. To link two different compounds in a step-wise manner, hetero-bifunctional cross-linkers can be used that eliminate unwanted homopolymer formation.

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1987). The use of such cross-linkers is well understood in the art. Another embodiment involves the use of flexible linkers.

U.S. Pat. No. 4,680,338 describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Particular uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

E. Multispecific Antibodies

In certain embodiments, antibodies of the present disclosure are bispecific or multispecific. Bispecific antibodies are antibodies that have binding specificities for at least two different epitopes. Exemplary bispecific antibodies may bind to two different epitopes of a single antigen. Other such antibodies may combine a first antigen binding site with a binding site for a second antigen. Alternatively, an anti-pathogen arm may be combined with an arm that binds to a triggering molecule on a leukocyte, such as a T-cell receptor molecule (e.g., CD3), or Fc receptors for IgG (FcγR), such as FcγRI (CD64), FcγRII (CD32) and Fc gamma RIII (CD16), so as to focus and localize cellular defense mechanisms to the infected cell. Bispecific antibodies may also be used to localize cytotoxic agents to infected cells. These antibodies possess a pathogen-binding arm and an arm that binds the cytotoxic agent (e.g., saporin, anti-interferon-α, vinca alkaloid, ricin A chain, methotrexate or radioactive isotope hapten). Bispecific antibodies can be prepared as full-length antibodies or antibody fragments (e.g., F(ab′)₂ bispecific antibodies). WO 96/16673 describes a bispecific anti-ErbB2/anti-Fc gamma RIII antibody and U.S. Pat. No. 5,837,234 discloses a bispecific anti-ErbB2/anti-Fc gamma RI antibody. A bispecific anti-ErbB2/Fc alpha antibody is shown in WO98/02463. U.S. Pat. No. 5,821,337 teaches a bispecific anti-ErbB2/anti-CD3 antibody.

Methods for making bispecific antibodies are known in the art. Traditional production of full-length bispecific antibodies is based on the co-expression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of ten different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable regions with the desired binding specificities (antibody-antigen combining sites) are fused to immunoglobulin constant domain sequences. Preferably, the fusion is with an Ig heavy chain constant domain, comprising at least part of the hinge, C_{H2}, and C_{H3} regions. It is preferred to have the first heavy-chain constant region (C_{H1}) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant effect on the yield of the desired chain combination.

In a particular embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H3} domain In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Techniques exist that facilitate the direct recovery of Fab′-SH fragments from E. coli, which can be chemically coupled to form bispecific antibodies. Shalaby et al., J. Exp. Med., 175: 217-225 (1992) describe the production of a humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described (Merchant et al., Nat. Biotechnol. 16, 677-681 (1998) doi:10.1038/nbt0798-677pmid:9661204). For example, bispecific antibodies have been produced using leucine zippers (Kostelny et al., J. Immunol., 148(5):1547-1553, 1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a V_{H} connected to a V_{L} by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

In a particular embodiment, a bispecific or multispecific antibody may be formed as a DOCK-AND-LOCK™ (DNL™) complex (see, e.g., U.S. Pat. Nos. 7,521,056; 7,527,787; 7,534,866; 7,550,143 and 7,666,400, the Examples section of each of which is incorporated herein by reference.) Generally, the technique takes advantage of the specific and high-affinity binding interactions that occur between a dimerization and docking domain (DDD) sequence of the regulatory (R) subunits of cAMP-dependent protein kinase (PKA) and an anchor domain (AD) sequence derived from any of a variety of AKAP proteins (Baillie et al., FEBS Letters. 2005; 579: 3264; Wong and Scott, Nat. Rev. Mol. Cell Biol. 2004; 5: 959). The DDD and AD peptides may be attached to any protein, peptide or other molecule. Because the DDD sequences spontaneously dimerize and bind to the AD sequence, the technique allows the formation of complexes between any selected molecules that may be attached to DDD or AD sequences.

Antibodies with more than two valencies are contemplated. For example, trispecific antibodies can be prepared (Tutt et al., J. Immunol. 147: 60, 1991; Xu et al., Science, 358(6359):85-90, 2017). A multivalent antibody may be internalized (and/or catabolized) faster than a bivalent antibody by a cell expressing an antigen to which the antibodies bind. The antibodies of the present disclosure can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable regions. For instance, the polypeptide chain(s) may comprise VD1-(X1)ₙ-VD2-(X2)ₙ-Fc, wherein VD1 is a first variable region, VD2 is a second variable region, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein preferably further comprises at least two (and preferably four) light chain variable region polypeptides. The multivalent antibody herein may, for instance, comprise from about two to about eight light chain variable region polypeptides. The light chain variable region polypeptides contemplated here comprise a light chain variable region and, optionally, further comprise a C_{L} domain.

Charge modifications are particularly useful in the context of a multispecific antibody, where amino acid substitutions in Fab molecules result in reducing the mispairing of light chains with non-matching heavy chains (Bence-Jones-type side products), which can occur in the production of Fab-based bi-/multispecific antigen binding molecules with a VH/VL exchange in one (or more, in case of molecules comprising more than two antigen-binding Fab molecules) of their binding arms (see also PCT publication no. WO 2015/150447, particularly the examples therein, incorporated herein by reference in its entirety).

Accordingly, in particular embodiments, an antibody comprised in the therapeutic agent comprises
(a) a first Fab molecule which specifically binds to a first antigen
(b) a second Fab molecule which specifically binds to a second antigen, and wherein the variable domains VL and VH of the Fab light chain and the Fab heavy chain are replaced by each other,
wherein the first antigen is an activating T cell antigen and the second antigen is a target cell antigen, or the first antigen is a target cell antigen and the second antigen is an activating T cell antigen; and
wherein
i) in the constant domain CL of the first Fab molecule under a) the amino acid at position 124 is substituted by a positively charged amino acid (numbering according to Kabat), and wherein in the constant domain CH1 of the first Fab molecule under a) the amino acid at position 147 or the amino acid at position 213 is substituted by a negatively charged amino acid (numbering according to Kabat EU index); or
ii) in the constant domain CL of the second Fab molecule under b) the amino acid at position 124 is substituted by a positively charged amino acid (numbering according to Kabat), and wherein in the constant domain CH1 of the second Fab molecule under b) the amino acid at position 147 or the amino acid at position 213 is substituted by a negatively charged amino acid (numbering according to Kabat EU index).
   The antibody may not comprise both modifications mentioned under i) and ii). The constant domains CL and CH1 of the second Fab molecule are not replaced by each other (i.e., remain unexchanged).

In another embodiment of the antibody, in the constant domain CL of the first Fab molecule under a) the amino acid at position 124 is substituted independently by lysine (K), arginine (R) or histidine (H) (numbering according to Kabat) (in one preferred embodiment independently by lysine (K) or arginine (R)), and in the constant domain CH1 of the first Fab molecule under a) the amino acid at position 147 or the amino acid at position 213 is substituted independently by glutamic acid (E), or aspartic acid (D) (numbering according to Kabat EU index).

In a further embodiment, in the constant domain CL of the first Fab molecule under a) the amino acid at position 124 is substituted independently by lysine (K), arginine (R) or histidine (H) (numbering according to Kabat), and in the constant domain CH1 of the first Fab molecule under a) the amino acid at position 147 is substituted independently by glutamic acid (E), or aspartic acid (D) (numbering according to Kabat EU index).

In a particular embodiment, in the constant domain CL of the first Fab molecule under a) the amino acid at position 124 is substituted independently by lysine (K), arginine (R) or histidine (H) (numbering according to Kabat) (in one preferred embodiment independently by lysine (K) or arginine (R)) and the amino acid at position 123 is substituted independently by lysine (K), arginine (R) or histidine (H) (numbering according to Kabat) (in one preferred embodiment independently by lysine (K) or arginine (R)), and in the constant domain CH1 of the first Fab molecule under a) the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In a more particular embodiment, in the constant domain CL of the first Fab molecule under a) the amino acid at position 124 is substituted by lysine (K) (numbering according to Kabat) and the amino acid at position 123 is substituted by lysine (K) or arginine (R) (numbering according to Kabat), and in the constant domain CH1 of the first Fab molecule under a) the amino acid at position 147 is substituted by glutamic acid (E) (numbering according to Kabat EU index) and the amino acid at position 213 is substituted by glutamic acid (E) (numbering according to Kabat EU index).

In an even more particular embodiment, in the constant domain CL of the first Fab molecule under a) the amino acid at position 124 is substituted by lysine (K) (numbering according to Kabat) and the amino acid at position 123 is substituted by arginine (R) (numbering according to Kabat), and in the constant domain CH1 of the first Fab molecule under a) the amino acid at position 147 is substituted by glutamic acid (E) (numbering according to Kabat EU index) and the amino acid at position 213 is substituted by glutamic acid (E) (numbering according to Kabat EU index).

F. Chimeric Antigen Receptors

Artificial T cell receptors (also known as chimeric T cell receptors, chimeric immunoreceptors, chimeric antigen receptors (CARs)) are engineered receptors, which graft an arbitrary specificity onto an immune effector cell. Typically, these receptors are used to graft the specificity of a monoclonal antibody onto a T cell, with transfer of their coding sequence facilitated by retroviral vectors. In this way, a large number of target-specific T cells can be generated for adoptive cell transfer. Phase I clinical studies of this approach show efficacy.

The most common form of these molecules are fusions of single-chain variable fragments (scFv) derived from monoclonal antibodies, fused to CD3-zeta transmembrane and endodomain Such molecules result in the transmission of a zeta signal in response to recognition by the scFv of its target. An example of such a construct is 14g2a-Zeta, which is a fusion of a scFv derived from hybridoma 14g2a (which recognizes disialoganglioside GD2). When T cells express this molecule (usually achieved by oncoretroviral vector transduction), they recognize and kill target cells that express GD2 (e.g., neuroblastoma cells). To target malignant B cells, investigators have redirected the specificity of T cells using a chimeric immunoreceptor specific for the B-lineage molecule, CD19.

The variable portions of an immunoglobulin heavy and light chain are fused by a flexible linker to form a scFv. This scFv is preceded by a signal peptide to direct the nascent protein to the endoplasmic reticulum and subsequent surface expression (this is cleaved). A flexible spacer allows to the scFv to orient in different directions to enable antigen binding. The transmembrane domain is a typical hydrophobic alpha helix usually derived from the original molecule of the signaling endodomain which protrudes into the cell and transmits the desired signal.

Type I proteins are in fact two protein domains linked by a transmembrane alpha helix in between. The cell membrane lipid bilayer, through which the transmembrane domain passes, acts to isolate the inside portion (endodomain) from the external portion (ectodomain). It is not so surprising that attaching an ectodomain from one protein to an endodomain of another protein results in a molecule that combines the recognition of the former to the signal of the latter.

Ectodomain. A signal peptide directs the nascent protein into the endoplasmic reticulum. This is essential if the receptor is to be glycosylated and anchored in the cell membrane. Any eukaryotic signal peptide sequence usually works fine. Generally, the signal peptide natively attached to the amino-terminal most component is used (e.g., in a scFv with orientation light chain—linker—heavy chain, the native signal of the light-chain is used

The antigen recognition domain is usually an scFv. There are however many alternatives. An antigen recognition domain from native T-cell receptor (TCR) alpha and beta single chains have been described, as have simple ectodomains (e.g., CD4 ectodomain to recognize HIV infected cells) and more exotic recognition components such as a linked cytokine (which leads to recognition of cells bearing the cytokine receptor). In fact, almost anything that binds a given target with high affinity can be used as an antigen recognition region.

A spacer region links the antigen binding domain to the transmembrane domain. It should be flexible enough to allow the antigen binding domain to orient in different directions to facilitate antigen recognition. The simplest form is the hinge region from IgG1. Alternatives include the CH₂CH₃ region of immunoglobulin and portions of CD3. For most scFv based constructs, the IgG1 hinge suffices. However, the best spacer often has to be determined empirically.

Transmembrane domain. The transmembrane domain is a hydrophobic alpha helix that spans the membrane. Generally, the transmembrane domain from the most membrane proximal component of the endodomain is used. Interestingly, using the CD3-zeta transmembrane domain may result in incorporation of the artificial TCR into the native TCR a factor that is dependent on the presence of the native CD3-zeta transmembrane charged aspartic acid residue. Different transmembrane domains result in different receptor stability. The CD28 transmembrane domain results in a brightly expressed, stable receptor.

Endodomain. This is the “business-end” of the receptor. After antigen recognition, receptors cluster, and a signal is transmitted to the cell. The most commonly used endodomain component is CD3-zeta which contains 3 ITAMs. This transmits an activation signal to the T cell after antigen is bound. CD3-zeta may not provide a fully competent activation signal and additional co-stimulatory signaling is needed.

“First-generation” CARs typically had the intracellular domain from the CD3 ξ-chain, which is the primary transmitter of signals from endogenous TCRs. “Second-generation” CARs add intracellular signaling domains from various costimulatory protein receptors (e.g., CD28, 41BB, ICOS) to the cytoplasmic tail of the CAR to provide additional signals to the T cell. Preclinical studies have indicated that the second generation of CAR designs improves the antitumor activity of T cells. More recent, “third-generation” CARs combine multiple signaling domains, such as CD3z-CD28-41BB or CD3z-CD28-OX40, to further augment potency.

G. ADCs

Antibody Drug Conjugates or ADCs are a new class of highly potent biopharmaceutical drugs designed as a targeted therapy for the treatment of people with infectious disease. ADCs are complex molecules composed of an antibody (a whole mAb or an antibody fragment such as a single-chain variable fragment, or scFv) linked, via a stable chemical linker with labile bonds, to a biological active cytotoxic/anti-viral payload or drug. Antibody Drug Conjugates are examples of bioconjugates and immunoconjugates.

By combining the unique targeting capabilities of monoclonal antibodies with the cancer-killing ability of cytotoxic drugs, antibody-drug conjugates allow sensitive discrimination between healthy and diseased tissue. This means that, in contrast to traditional systemic approaches, antibody-drug conjugates target and attack the infected cell so that healthy cells are less severely affected.

In the development ADC-based anti-tumor therapies, an anticancer drug (e.g., a cell toxin or cytotoxin) is coupled to an antibody that specifically targets a certain cell marker (e.g., a protein that, ideally, is only to be found in or on infected cells). Antibodies track these proteins down in the body and attach themselves to the surface of cancer cells. The biochemical reaction between the antibody and the target protein (antigen) triggers a signal in the tumor cell, which then absorbs or internalizes the antibody together with the cytotoxin. After the ADC is internalized, the cytotoxic drug is released and kills the cell or impairs viral replication. Due to this targeting, ideally the drug has lower side effects and gives a wider therapeutic window than other agents.

A stable link between the antibody and cytotoxic/anti-viral agent is a crucial aspect of an ADC. Linkers are based on chemical motifs including disulfides, hydrazones or peptides (cleavable), or thioethers (noncleavable) and control the distribution and delivery of the cytotoxic agent to the target cell. Cleavable and noncleavable types of linkers have been proven to be safe in preclinical and clinical trials. Brentuximab vedotin includes an enzyme-sensitive cleavable linker that delivers the potent and highly toxic antimicrotubule agent Monomethyl auristatin E or MMAE, a synthetic antineoplastic agent, to human specific CD30-positive malignant cells. Because of its high toxicity MMAE, which inhibits cell division by blocking the polymerization of tubulin, cannot be used as a single-agent chemotherapeutic drug. However, the combination of MMAE linked to an anti-CD30 monoclonal antibody (cAC10, a cell membrane protein of the tumor necrosis factor or TNF receptor) proved to be stable in extracellular fluid, cleavable by cathepsin and safe for therapy. Trastuzumab emtansine, the other approved ADC, is a combination of the microtubule-formation inhibitor mertansine (DM-1), a derivative of the Maytansine, and antibody trastuzumab (Herceptin®/Genentech/Roche) attached by a stable, non-cleavable linker.

The availability of better and more stable linkers has changed the function of the chemical bond. The type of linker, cleavable or noncleavable, lends specific properties to the cytotoxic (anti-cancer) drug. For example, a non-cleavable linker keeps the drug within the cell. As a result, the entire antibody, linker and cytotoxic agent enter the targeted cancer cell where the antibody is degraded to the level of an amino acid. The resulting complex—amino acid, linker and cytotoxic agent—now becomes the active drug. In contrast, cleavable linkers are catalyzed by enzymes in the host cell where it releases the cytotoxic agent.

Another type of cleavable linker, currently in development, adds an extra molecule between the cytotoxic/anti-viral drug and the cleavage site. This linker technology allows researchers to create ADCs with more flexibility without worrying about changing cleavage kinetics. Researchers are also developing a new method of peptide cleavage based on Edman degradation, a method of sequencing amino acids in a peptide. Future direction in the development of ADCs also include the development of site-specific conjugation (TDCs) to further improve stability and therapeutic index and a emitting immunoconjugates and antibody-conjugated nanoparticles.

H. BiTES

Bi-specific T-cell engagers (BiTEs) are a class of artificial bispecific monoclonal antibodies that are investigated for the use as anti-cancer drugs. They direct a host's immune system, more specifically the T cells' cytotoxic activity, against infected cells. BiTE is a registered trademark of Micromet AG.

BiTEs are fusion proteins consisting of two single-chain variable fragments (scFvs) of different antibodies, or amino acid sequences from four different genes, on a single peptide chain of about 55 kilodaltons. One of the scFvs binds to T cells via the CD3 receptor, and the other to an infected cell via a specific molecule.

Like other bispecific antibodies, and unlike ordinary monoclonal antibodies, BiTEs form a link between T cells and target cells. This causes T cells to exert cytotoxic/anti-viral activity on infected cells by producing proteins like perforin and granzymes, independently of the presence of MHC I or co-stimulatory molecules. These proteins enter infected cells and initiate the cell's apoptosis. This action mimics physiological processes observed during T cell attacks against infected cells.

I. Intrabodies

In a particular embodiment, the antibody is a recombinant antibody that is suitable for action inside of a cell—such antibodies are known as “intrabodies.” These antibodies may interfere with target function by a variety of mechanism, such as by altering intracellular protein trafficking, interfering with enzymatic function, and blocking protein-protein or protein-DNA interactions. In many ways, their structures mimic or parallel those of single chain and single domain antibodies, discussed above. Indeed, single-transcript/single-chain is an important feature that permits intracellular expression in a target cell, and also makes protein transit across cell membranes more feasible. However, additional features are required.

The two major issues impacting the implementation of intrabody therapeutic are delivery, including cell/tissue targeting, and stability. With respect to delivery, a variety of approaches have been employed, such as tissue-directed delivery, use of cell-type specific promoters, viral-based delivery and use of cell-permeability/membrane translocating peptides. With respect to the stability, the approach is generally to either screen by brute force, including methods that involve phage display and may include sequence maturation or development of consensus sequences, or more directed modifications such as insertion stabilizing sequences (e.g., Fc regions, chaperone protein sequences, leucine zippers) and disulfide replacement/modification.

An additional feature that intrabodies may require is a signal for intracellular targeting. Vectors that can target intrabodies (or other proteins) to subcellular regions such as the cytoplasm, nucleus, mitochondria and ER have been designed and are commercially available (Invitrogen Corp.; Persic et al., 1997).

By virtue of their ability to enter cells, intrabodies have additional uses that other types of antibodies may not achieve. In the case of the present antibodies, the ability to interact with the MUC1 cytoplasmic domain in a living cell may interfere with functions associated with the MUC1 CD, such as signaling functions (binding to other molecules) or oligomer formation. In particular, it is contemplated that such antibodies can be used to inhibit MUC1 dimer formation.

J. Purification

In certain embodiments, the antibodies of the present disclosure may be purified. The term “purified,” as used herein, is intended to refer to a composition, isolatable from other components, wherein the protein is purified to any degree relative to its naturally-obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an antibody of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fc portion of the antibody. Alternatively, antigens may be used to simultaneously purify and select appropriate antibodies. Such methods often utilize the selection agent bound to a support, such as a column, filter or bead. The antibodies are bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

### III. ACTIVE/PASSIVE IMMUNIZATION AND TREATMENT/PREVENTION OF DENGUE VIRUS INFECTION

A. Formulation and Administration

The present disclosure provides pharmaceutical compositions comprising anti-Dengue virus antibodies and antigens for generating the same. Such compositions comprise a prophylactically or therapeutically effective amount of an antibody or a fragment thereof, or a peptide immunogen, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, intra-rectal, vaginal, topical or delivered by mechanical ventilation.

Active vaccines are also envisioned where antibodies like those disclosed are produced in vivo in a subject at risk of dengue virus infection. Such vaccines can be formulated for parenteral administration, e.g., formulated for injection via the intradermal, intravenous, intramuscular, subcutaneous, or even intraperitoneal routes Administration by intradermal and intramuscular routes are contemplated. The vaccine could alternatively be administered by a topical route directly to the mucosa, for example by nasal drops, inhalation, by nebulizer, or via intrarectal or vaginal delivery. Pharmaceutically acceptable salts include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Passive transfer of antibodies, known as artificially acquired passive immunity, generally will involve the use of intravenous or intramuscular injections. The forms of antibody can be human or animal blood plasma or serum, as pooled human immunoglobulin for intravenous (IVIG) or intramuscular (IG) use, as high-titer human IVIG or IG from immunized or from donors recovering from disease, and as monoclonal antibodies (mAbs). Such immunity generally lasts for only a short period of time, and there is also a potential risk for hypersensitivity reactions, and serum sickness, especially from gamma globulin of non-human origin. However, passive immunity provides immediate protection. The antibodies will be formulated in a carrier suitable for injection, i.e., sterile and syringeable.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampule of sterile water for injection or saline can be provided so that the ingredients may be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

2. ADCC

Antibody-dependent cell-mediated cytotoxicity (ADCC) is an immune mechanism leading to the lysis of antibody-coated target cells by immune effector cells. The target cells are cells to which antibodies or fragments thereof comprising an Fc region specifically bind, generally via the protein part that is N-terminal to the Fc region. By “antibody having increased/reduced antibody dependent cell-mediated cytotoxicity (ADCC)” is meant an antibody having increased/reduced ADCC as determined by any suitable method known to those of ordinary skill in the art.

As used herein, the term “increased/reduced ADCC” is defined as either an increase/reduction in the number of target cells that are lysed in a given time, at a given concentration of antibody in the medium surrounding the target cells, by the mechanism of ADCC defined above, and/or a reduction/increase in the concentration of antibody, in the medium surrounding the target cells, required to achieve the lysis of a given number of target cells in a given time, by the mechanism of ADCC. The increase/reduction in ADCC is relative to the ADCC mediated by the same antibody produced by the same type of host cells, using the same standard production, purification, formulation and storage methods (which are known to those skilled in the art), but that has not been engineered. For example, the increase in ADCC mediated by an antibody produced by host cells engineered to have an altered pattern of glycosylation (e.g., to express the glycosyltransferase, GnTIII, or other glycosyltransferases) by the methods described herein, is relative to the ADCC mediated by the same antibody produced by the same type of non-engineered host cells.

3. CDC

Complement-dependent cytotoxicity (CDC) is a function of the complement system. It is the processes in the immune system that kill pathogens by damaging their membranes without the involvement of antibodies or cells of the immune system. There are three main processes. All three insert one or more membrane attack complexes (MAC) into the pathogen which cause lethal colloid-osmotic swelling, i.e., CDC. It is one of the mechanisms by which antibodies or antibody fragments have an anti-viral effect.

### IV. ANTIBODY CONJUGATES

Antibodies of the present disclosure may be linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radionuclides, antiviral agents, chelating agents, cytokines, growth factors, and oligo- or polynucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, photoaffinity molecules, colored particles or ligands, such as biotin.

Antibody conjugates are generally preferred for use as diagnostic agents. Antibody diagnostics generally fall within two classes, those for use in in vitro diagnostics, such as in a variety of immunoassays, and those for use in vivo diagnostic protocols, generally known as “antibody-directed imaging.” Many appropriate imaging agents are known in the art, as are methods for their attachment to antibodies (see, for e.g., U.S. Pat. Nos. 5,021,236, 4,938,948, and 4,472,509). The imaging moieties used can be paramagnetic ions, radioactive isotopes, fluorochromes, NMR-detectable substances, and X-ray imaging agents.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰, ¹²⁵I is often being preferred for use in certain embodiments, and technicium⁹⁹m and/or indium¹¹¹ are also often preferred due to their low energy and suitability for long range detection. Radioactively labeled monoclonal antibodies of the present disclosure may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the disclosure may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Additional types of antibodies contemplated in the present disclosure are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and avidin and streptavidin compounds. The use of such labels is well known to those of skill in the art and are described, for example, in U.S. Pat. 3,817,837, 3,850,752, 3,939,350, 3,996,345, 4,277,437, 4,275,149 and 4,366,241.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter and Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

### V. IMMUNODETECTION METHODS

In still further embodiments, the present disclosure concerns immunodetection methods for binding, purifying, removing, quantifying and otherwise generally detecting dengue virus and its associated antigens. While such methods can be applied in a traditional sense, another use will be in quality control and monitoring of vaccine and other virus stocks, where antibodies according to the present disclosure can be used to assess the amount or integrity (i.e. , long term stability) of antigens in viruses. Alternatively, the methods may be used to screen various antibodies for appropriate/desired reactivity profiles.

Other immunodetection methods include specific assays for determining the presence of Dengue virus in a subject. A wide variety of assay formats are contemplated, but specifically those that would be used to detect dengue virus in a fluid obtained from a subject, such as saliva, blood, plasma, sputum, semen or urine. In particular, semen has been demonstrated as a viable sample for detecting other viruses (Purpura et al., 2016; Mansuy et al., 2016; Barzon et al., 2016; Gornet et al., 2016; Duffy et al., 2009; CDC, 2016; Halfon et al., 2010; Elder et al. 2005). The assays may be advantageously formatted for non-healthcare (home) use, including lateral flow assays (see below) analogous to home pregnancy tests. These assays may be packaged in the form of a kit with appropriate reagents and instructions to permit use by the subject of a family member.

Some immunodetection methods include enzyme linked immunosorbent assay (ELISA), radioimmunoassay (RIA), immunoradiometric assay, fluoroimmunoassay, chemiluminescent assay, bioluminescent assay, and western blot to mention a few. In particular, a competitive assay for the detection and quantitation of dengue virus antibodies directed to specific parasite epitopes in samples also is provided. The steps of various useful immunodetection methods have been described in the scientific literature, such as, e.g., Doolittle and Ben-Zeev (1999), Gulbis and Galand (1993), De Jager et al. (1993), and Nakamura et al. (1987). In general, the immunobinding methods include obtaining a sample suspected of containing dengue virus and contacting the sample with a first antibody in accordance with the present disclosure, as the case may be, under conditions effective to allow the formation of immunocomplexes.

These methods include methods for purifying dengue virus or related antigens from a sample. The antibody will preferably be linked to a solid support, such as in the form of a column matrix, and the sample suspected of containing the dengue virus or antigenic component will be applied to the immobilized antibody. The unwanted components will be washed from the column, leaving the dengue virus antigen immunocomplexed to the immobilized antibody, which is then collected by removing the organism or antigen from the column.

The immunobinding methods also include methods for detecting and quantifying the amount of dengue virus or related components in a sample and the detection and quantification of any immune complexes formed during the binding process. Here, one would obtain a sample suspected of containing dengue virus or its antigens and contact the sample with an antibody that binds dengue virus or components thereof, followed by detecting and quantifying the amount of immune complexes formed under the specific conditions. In terms of antigen detection, the biological sample analyzed may be any sample that is suspected of containing dengue virus or dengue virus antigen, such as a tissue section or specimen, a homogenized tissue extract, a biological fluid, including blood and serum, or a secretion, such as feces or urine.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to dengue virus or antigens present. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837, 3,850,752, 3,939,350, 3,996,345, 4,277,437, 4,275,149 and 4,366,241. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody that has binding affinity for the antibody, is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first biotinylated antibody is used to detect the target antigen, and a second antibody is then used to detect the biotin attached to the complexed biotin. In that method, the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

A. ELISAs

Immunoassays, in their most simple and direct sense, are binding assays. Certain preferred immunoassays are the various types of enzyme linked immunosorbent assays (ELISAs) and radioimmunoassays (RIA) known in the art Immunohistochemical detection using tissue sections is also particularly useful. However, it will be readily appreciated that detection is not limited to such techniques, and western blotting, dot blotting, FACS analyses, and the like may also be used.

In one exemplary ELISA, the antibodies of the disclosure are immobilized onto a selected surface exhibiting protein affinity, such as a well in a polystyrene microtiter plate. Then, a test composition suspected of containing the dengue virus or dengue virus antigen is added to the wells. After binding and washing to remove non-specifically bound immune complexes, the bound antigen may be detected. Detection may be achieved by the addition of another anti-Dengue virus antibody that is linked to a detectable label. This type of ELISA is a simple “sandwich ELISA.” Detection may also be achieved by the addition of a second anti-dengue virus antibody, followed by the addition of a third antibody that has binding affinity for the second antibody, with the third antibody being linked to a detectable label.

In another exemplary ELISA, the samples suspected of containing the dengue virus or dengue virus antigen are immobilized onto the well surface and then contacted with the anti-dengue virus antibodies of the disclosure. After binding and washing to remove non-specifically bound immune complexes, the bound anti-dengue virus antibodies are detected. Where the initial anti-Dengue virus antibodies are linked to a detectable label, the immune complexes may be detected directly. Again, the immune complexes may be detected using a second antibody that has binding affinity for the first anti-dengue virus antibody, with the second antibody being linked to a detectable label.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C. or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

In another embodiment, the present disclosure contemplates the use of competitive formats. This is particularly useful in the detection of dengue virus antibodies in sample. In competition-based assays, an unknown amount of analyte or antibody is determined by its ability to displace a known amount of labeled antibody or analyte. Thus, the quantifiable loss of a signal is an indication of the amount of unknown antibody or analyte in a sample.

Here, the inventor proposes the use of labeled dengue virus monoclonal antibodies to determine the amount of dengue virus antibodies in a sample. The basic format would include contacting a known amount of dengue virus monoclonal antibody (linked to a detectable label) with dengue virus antigen or particle. The dengue virus antigen or organism is preferably attached to a support. After binding of the labeled monoclonal antibody to the support, the sample is added and incubated under conditions permitting any unlabeled antibody in the sample to compete with, and hence displace, the labeled monoclonal antibody. By measuring either the lost label or the label remaining (and subtracting that from the original amount of bound label), one can determine how much non-labeled antibody is bound to the support, and thus how much antibody was present in the sample.

B. Western blot

The western blot (alternatively, protein immunoblot) is an analytical technique used to detect specific proteins in a given sample of tissue homogenate or extract. It uses gel electrophoresis to separate native or denatured proteins by the length of the polypeptide (denaturing conditions) or by the 3-D structure of the protein (native/non-denaturing conditions). The proteins are then transferred to a membrane (typically nitrocellulose or PVDF), where they are probed (detected) using antibodies specific to the target protein.

Samples may be taken from whole tissue or from cell culture. In most cases, solid tissues are first broken down mechanically using a blender (for larger sample volumes), using a homogenizer (smaller volumes), or by sonication. Cells may also be broken open by one of the above mechanical methods. However, it should be noted that bacteria, virus or environmental samples can be the source of protein and thus Western blotting is not restricted to cellular studies only. Assorted detergents, salts, and buffers may be employed to encourage lysis of cells and to solubilize proteins. Protease and phosphatase inhibitors are often added to prevent the digestion of the sample by its own enzymes. Tissue preparation is often done at cold temperatures to avoid protein denaturing.

The proteins of the sample are separated using gel electrophoresis. Separation of proteins may be by isoelectric point (pI), molecular weight, electric charge, or a combination of these factors. The nature of the separation depends on the treatment of the sample and the nature of the gel. This is a very useful way to determine a protein. It is also possible to use a two-dimensional (2-D) gel which spreads the proteins from a single sample out in two dimensions. Proteins are separated according to isoelectric point (pH at which they have neutral net charge) in the first dimension, and according to their molecular weight in the second dimension.

In order to make the proteins accessible to antibody detection, they are moved from within the gel onto a membrane made of nitrocellulose or polyvinylidene difluoride (PVDF). The membrane is placed on top of the gel, and a stack of filter papers placed on top of that. The entire stack is placed in a buffer solution which moves up the paper by capillary action, bringing the proteins with it. Another method for transferring the proteins is called electroblotting and uses an electric current to pull proteins from the gel into the PVDF or nitrocellulose membrane. The proteins move from within the gel onto the membrane while maintaining the organization they had within the gel. As a result of this blotting process, the proteins are exposed on a thin surface layer for detection (see below). Both varieties of membrane are chosen for their non-specific protein binding properties (i.e., binds all proteins equally well). Protein binding is based upon hydrophobic interactions, as well as charged interactions between the membrane and protein. Nitrocellulose membranes are cheaper than PVDF but are far more fragile and do not stand up well to repeated probings. The uniformity and overall effectiveness of transfer of protein from the gel to the membrane can be checked by staining the membrane with Coomassie Brilliant Blue or Ponceau S dyes. Once transferred, proteins are detected using labeled primary antibodies, or unlabeled primary antibodies followed by indirect detection using labeled protein A or secondary labeled antibodies binding to the Fc region of the primary antibodies.

C. Lateral Flow Assays

Lateral flow assays, also known as lateral flow immunochromatographic assays, are simple devices intended to detect the presence (or absence) of a target analyte in sample (matrix) without the need for specialized and costly equipment, though many laboratory-based applications exist that are supported by reading equipment. Typically, these tests are used as low resources medical diagnostics, either for home testing, point of care testing, or laboratory use. A widely spread and well-known application is the home pregnancy test.

The technology is based on a series of capillary beds, such as pieces of porous paper or sintered polymer. Each of these elements has the capacity to transport fluid (e.g., urine) spontaneously. The first element (the sample pad) acts as a sponge and holds an excess of sample fluid. Once soaked, the fluid migrates to the second element (conjugate pad) in which the manufacturer has stored the so-called conjugate, a dried format of bio-active particles (see below) in a salt-sugar matrix that contains everything to guarantee an optimized chemical reaction between the target molecule (e.g., an antigen) and its chemical partner (e.g., antibody) that has been immobilized on the particle's surface. While the sample fluid dissolves the salt-sugar matrix, it also dissolves the particles and in one combined transport action the sample and conjugate mix while flowing through the porous structure. In this way, the analyte binds to the particles while migrating further through the third capillary bed. This material has one or more areas (often called stripes) where a third molecule has been immobilized by the manufacturer. By the time the sample-conjugate mix reaches these strips, analyte has been bound on the particle and the third ‘capture’ molecule binds the complex. After a while, when more and more fluid has passed the stripes, particles accumulate and the stripe-area changes color. Typically, there are at least two stripes: one (the control) that captures any particle and thereby shows that reaction conditions and technology worked fine, the second contains a specific capture molecule and only captures those particles onto which an analyte molecule has been immobilized. After passing these reaction zones, the fluid enters the final porous material—the wick—that simply acts as a waste container. Lateral Flow Tests can operate as either competitive or sandwich assays. Lateral flow assays are disclosed in U.S. Pat. No. 6,485,982.

D. Immunohistochemistry

The antibodies of the present disclosure may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors and is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections from the capsule. Alternatively, whole frozen tissue samples may be used for serial section cuttings.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections. Again, whole tissue samples may be substituted.

E. Immunodetection Kits

In still further embodiments, the present disclosure concerns immunodetection kits for use with the immunodetection methods described above. As the antibodies may be used to detect Dengue virus or Dengue virus antigens, the antibodies may be included in the kit. The immunodetection kits will thus comprise, in suitable container means, a first antibody that binds to Dengue virus or Dengue virus antigen, and optionally an immunodetection reagent.

In certain embodiments, the Dengue virus antibody may be pre-bound to a solid support, such as a column matrix and/or well of a microtiter plate. The immunodetection reagents of the kit may take any one of a variety of forms, including those detectable labels that are associated with or linked to the given antibody. Detectable labels that are associated with or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and all such labels may be employed in connection with the present disclosure.

The kits may further comprise a suitably aliquoted composition of the Dengue virus or Dengue virus antigens, whether labeled or unlabeled, as may be used to prepare a standard curve for a detection assay. The kits may contain antibody-label conjugates either in fully conjugated form, in the form of intermediates, or as separate moieties to be conjugated by the user of the kit. The components of the kits may be packaged either in aqueous media or in lyophilized form.

The container means of the kits will generally include at least one vial, test tube, flask, bottle, syringe or other container means, into which the antibody may be placed, or preferably, suitably aliquoted. The kits of the present disclosure will also typically include a means for containing the antibody, antigen, and any other reagent containers in close confinement for commercial sale. Such containers may include injection or blow-molded plastic containers into which the desired vials are retained.

F. Vaccine and Antigen Quality Control Assays

The present disclosure also contemplates the use of antibodies and antibody fragments as described herein for use in assessing the antigenic integrity of a viral antigen in a sample. Biological medicinal products like vaccines differ from chemical drugs in that they cannot normally be characterized molecularly; antibodies are large molecules of significant complexity and have the capacity to vary widely from preparation to preparation. They are also administered to healthy individuals, including children at the start of their lives, and thus a strong emphasis must be placed on their quality to ensure, to the greatest extent possible, that they are efficacious in preventing or treating life-threatening disease, without themselves causing harm.

The increasing globalization in the production and distribution of vaccines has opened new possibilities to better manage public health concerns but has also raised questions about the equivalence and interchangeability of vaccines procured across a variety of sources. International standardization of starting materials, of production and quality control testing, and the setting of high expectations for regulatory oversight on the way these products are manufactured and used, have thus been the cornerstone for continued success. But it remains a field in constant change, and continuous technical advances in the field offer a promise of developing potent new weapons against the oldest public health threats, as well as new ones—malaria, pandemic influenza, and HIV, to name a few—but also put a great pressure on manufacturers, regulatory authorities, and the wider medical community to ensure that products continue to meet the highest standards of quality attainable.

Thus, one may obtain an antigen or vaccine from any source or at any point during a manufacturing process. The quality control processes may therefore begin with preparing a sample for an immunoassay that identifies binding of an antibody or fragment disclosed herein to a viral antigen. Such immunoassays are disclosed elsewhere in this document, and any of these may be used to assess the structural/antigenic integrity of the antigen. Standards for finding the sample to contain acceptable amounts of antigenically correct and intact antigen may be established by regulatory agencies.

Another important embodiment where antigen integrity is assessed is in determining shelf-life and storage stability. Most medicines, including vaccines, can deteriorate over time. Therefore, it is critical to determine whether, over time, the degree to which an antigen, such as in a vaccine, degrades or destabilizes such that is it no longer antigenic and/or capable of generating an immune response when administered to a subject. Again, standards for finding the sample to contain acceptable amounts of antigenically intact antigen may be established by regulatory agencies.

In certain embodiments, viral antigens may contain more than one protective epitope. In these cases, it may prove useful to employ assays that look at the binding of more than one antibody, such as 2, 3, 4, 5 or even more antibodies. These antibodies bind to closely related epitopes, such that they are adjacent or even overlap each other. On the other hand, they may represent distinct epitopes from disparate parts of the antigen. By examining the integrity of multiple epitopes, a more complete picture of the antigen's overall integrity, and hence ability to generate a protective immune response, may be determined.

Antibodies and fragments thereof as described in the present disclosure may also be used in a kit for monitoring the efficacy of vaccination procedures by detecting the presence of protective Dengue virus antibodies. Antibodies, antibody fragment, or variants and derivatives thereof, as described in the present disclosure may also be used in a kit for monitoring vaccine manufacture with the desired immunogenicity.

### VI. EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Materials and Methods

Subjects. The Pediatric Dengue Cohort Study is an ongoing prospective dengue cohort study that follows approximately 3,700 children ages 2-14 in District II of Managua, Nicaragua. The protocol for the Pediatric Dengue Cohort Study in Nicaragua was reviewed and approved by the Institutional Review Boards of the University of California, Berkeley, (#2010-09-2245) and the Nicaraguan Ministry of Health (NIC-MINSA/CNDR-CIRE-09/03/07-008.ver1;). Parents or legal guardian of the subjects enrolled in the study provided written informed consent, and participants 6 years of age and older provided assent.

DENV infection and infection history. A suspected dengue case was considered a symptomatic DENV infection when 1) DENV RNA was detected by reverse-transcriptase polymerase chain reaction (RT-PCR)(Balmaseda et al., 1999; Lanciotti et al., 1992), 2) DENV was isolated(Balmaseda et al., 1999), 3) seroconversion was observed in paired acute and convalescent phase sera by IgM capture ELISA(Balmaseda et al., 2003; Balmaseda et al., 1999), or 4) seroconversion and/or a ≥4-fold increase in total DENV-specific antibody titer in paired acute and convalescent sera was observed by Inhibition ELISA(Balmaseda et al., 2006; Fernandez and Vazquez, 1990). Inapparent DENV infections were identified through serological testing of paired annual blood draws from healthy subjects (Balmaseda et al., 2010; Kuan et al,. 2009). Participants whose paired annual samples demonstrated seroconversion or a 4-fold or greater increase in total DENV-specific antibody titer by Inhibition ELISA (iELISA), but who had not experienced a documented febrile episode associated with acute DENV infection, were considered to have experienced an inapparent DENV infection (Balmaseda et al., 2010; Kuan et al., 2009)

One hundred and sixteen participants who entered the cohort dengue-naïve and had experienced at least two DENV infections as determined by the iELISA were selected, and neutralizing antibody titers (NT₅₀) for all four DENV serotypes at each annual sample were determined using a flow cytometry-based assay with reporter viral particles (RVPs) representing the four serotypes and Raji cells expressing the DENV attachment factor DC-SIGN(Mattia et al., 2011; Montoya et al., 2013). PBMC samples collected after the second DENV infection for three such participants—985, 1791 and 3242—were used for isolation of DENV3-specific hmAbs. All individuals experienced one symptomatic RT-PCR-confirmed DENV3 infection. Individuals 985 and 1791 experienced primary inapparent DENV2 infection followed by secondary DENV3 infection, while individual 3242 experienced primary DENV3 infection followed by secondary inapparent DENV1 infection (FIGS. 1A-D, Table B).

PBMC preparation. For PBMC preparation, blood samples were collected in Vacutainer tubes (Becton—Dickenson) with 5 mM EDTA as anticoagulant. Upon receipt at the Nicaraguan National Virology Laboratory, ˜5 ml of blood was transferred into a Leucosep tube (Greiner Bio-One) containing 3 ml of Ficoll Histopaque (Sigma) and centrifuged at 500 g for 20 minutes at room temperature. The PBMC fraction was collected and transferred to a tube containing 9 ml of PBS with 2% fetal bovine serum (FBS; Denville Scientific) and 1% penicillin/streptomycin (Sigma). Cells were washed and pelleted three times by centrifugation at 500 g for 10 min and resuspended in RPMI 1640 complete medium (RPMI 1640, 10% FBS, 1% GlutaMAX™, 1% HEPES and 1% penicillin/streptomycin). Before the third wash, cells were counted using a hemocytometer (Sismex XS-1000i). After the third wash, cells were resuspended in cryovials at a concentration of 3×10⁶ cells/ml in freezing medium (90% FBS, 10% dimethyl sulfoxide) and were placed in isopropanol containers (Mr. Frosty, Nalgene) at −80° C. overnight and transferred to liquid nitrogen for storage (Michlmayr et al., 2017; Zompi et al., 2012).

Generation of DENV3-specific hMAbs. Previously cryopreserved peripheral blood mononuclear cells (PBMCs) were thawed rapidly in a 37° C. water bath and washed prior to transformation with Epstein-Barr virus (EBV) and incubated with CpG and additional supplements, as described previously (Yu et al., 2008). Cultures were incubated at 37° C. in 5% CO₂ for 10 days prior to screening for DENV3-reactive cell lines with ELISA. The transformed B cell culture supernatants were screened by live virus capture ELISA for binding to a representative strain from each of the four DENV serotypes. The minimal frequency of DENV3-reactive B cells was estimated on the basis of the number of wells with DENV3-reactive supernatants as compared to the total number of lymphoblastoid cell line colonies in the transformation plates, as follows: [number of wells with DENV3-reactive supernatants]/[number of LCL colonies in the plate]. On the basis of the number of DENV positive wells and the number of transformed B cells tested (determined by average colony counts in transformed wells), the percentages of DENV E protein-reactive B cells in circulation were estimated to be 1.8, 1.1 and 1.6% of transformable B cells for subjects 3243, 985, and 1791, respectively, which were similar to B cell frequencies reported in earlier studies for DENV-immune adult subjects (Smith et al., 2014). Cells from wells with supernatants reacting in the DENV3 capture ELISA were subjected to cytofusion with HMMA2.5 non-secreting myeloma cells, as previously described (Smith et al., 2013; Smith et al., 2012). Following cytofusion, hybridomas were selected for growth in HAT medium containing ouabain. Wells containing hybridomas producing DENV3-reactive antibodies were cloned biologically by limiting dilution plating followed by flow cytometric sorting for single cells using a FACSAria III cell sorter (BDBiosciences). Once clonal, the cell lines were used to produce mAb immunoglobulin G (IgG) in cell supernatants, using serum-free medium, followed by protein G column purification. One hybridoma line (DENV-419) secreted poorly, so the inventor generated a recombinant form of the antibody and expressed it in mammalian cells prior to protein G column purification. Here, in all three subjects, the inventor focused on those antibodies that were TS against DENV3. This included 3 antibodies originating from subject 3243 (primary DENV3 infection; sample collected after secondary DENV infection) and 5 or 7 antibodies from subjects 1791 or 985, respectively, who had experienced DENV3 as a secondary DENV infection after primary DENV2.

Virus, rE and rEDIII ELISA. To evaluate if the oligomeric state of the E protein influences the binding efficiency of the mAbs, the inventor subjected the mAbs to an antigen-capture ELISA using DENV3 recombinant E (rE) proteins. DENV rE proteins exist in a concentration- and temperature-dependent monomer-to-dimer equilibrium (Kudlacek et al., 2018). At physiological conditions, rE is mainly present as a monomer (rE^{M}). Stable DENV3 homodimers (rE^{D}) were generated by introducing a disulfide interaction at the EDII-dimer interface (A257C). Ni²⁺-coated ELISA plates (Pierce Thermo) were coated with 5 ng/μL DENV3 rE^{M} or rE^{D} for 1 hour at 37° C. Next, the plates were blocked with TBS+0.05% Tween-20+3% skim milk for 1 hour at 37° C. Plates subsequently were washed three times with TBS+0.2% Tween-20 and incubated with serially diluted mAb (2-0.015 ng/μL) for 1 hour at 37° C. Next, plates were washed and incubated with 1:2,500 diluted alkaline-phosphatase (AP) conjugated anti-human IgG (Sigma) for 45 minutes at 37° C. After washing, wells were developed with AP substrate (Sigma) and absorbance was measured at 405 nm wavelength (Gallichotte et al., 2015).

Cell lines and viruses. Vero-81 cells (ATCC# CCL-81) were maintained in Dulbecco's modified Eagle's/Ham's F-12 50/50 Mix (DMEM/F-12 50/50) supplemented with non-essential amino acids (NEAA), glutamine and sodium bicarbonate (Vero cell medium) at 37° C. C6/36 cells (ATCC CRL-1660) were maintained in Gibco minimal essential medium (MEM) supplemented with 1% NEAA at 32° C. Both media were supplemented with 5% fetal bovine serum (FBS) and penicillin/streptomycin antibiotics. U937 cells expressing DC-SIGN (dendritic cell-specific intercellular adhesion molecule-3-grabbing nonintegrin), a known DENV attachment factor, were maintained as suspension cell cultures at 37° C. with 5% CO₂ in RPMI 1640 (Gibco) supplemented with 1% non-essential amino acids, 1% penicillin and streptomycin, and 5% fetal bovine serum (FBS; HyClone). The rDENV1 clone is based on DENV strain West Pac 74, the rDENV2 clone is based on DENV strain S16803, the rDENV3 clone is based on a Sri Lankan 1989 DENV strain and the DENV4 molecular clone was based on the sequence of Sri Lankan DENV strain 1992a, and have been previously described(Gallichotte et al., 2017; Gallichotte et al., 2015; Messer et al., 2012). The chimeric infectious clone rDENV4/3 M16 and the genotype panel of DENV3 also have been described previously(Widman et al., 2017).

Generation of the rDENV3/1and rDENV1/3 recombinant virus panels. The generation of a DENV3/1 chimera, designated rDENV3/1 ED-1A, was described previously (Messer et al., 2016). A four-component cDNA cloning system was used in which the DENV genome is divided into four segments that can be replicated separately as plasmids in Escherichia coli cells. Purified plasmids are cut with designated restriction enzymes to yield unique type IIS restriction endonuclease cleavage sites that can be ligated simultaneously to yield full-length DENV genome cDNA. A built-in T7 site is used to generate RNA, which is electroporated into C6/36 or Vero-81 cells to recover virus. Virus harvested from medium is subsequently passaged and sequence verified. To generate several additional chimeric rDENV3/1 viruses, the numbers and/or locations of amino acid residues from EDI and EDIII that were transplanted into DENV3 from DENV1 was increased systematically. A closely matched derivative called DENV3/1 EDI-B (23 residues) was isolated, which extended the original DENV3/1 EDI-A transplanted region to include two residues in EDIII of the neighboring protomer (e.g., D384E and N385K), removed one DENV1 residue from the EDI/II hinge region (N52Q), removed one DENV1 residue from the interior of EDI (V141I) and corrected a tissue-culture-induced mutation at residue F46L. The design of the DENV3/1 EDI/III-C chimeric virus further reduced the number of transplanted residues in the EDI/II hinge region by 3 residues (V50A, P53L and V55T), but converted most of the DENV3 ED I and ED III domains to DENV1, thereby increasing the total number of transplanted residues to 35 amino acids (Table A-b). The final derivative, designated the DENV3/1 EDI/III-D chimera, builds upon the DENV3/1 EDI/III-C backbone by converting an additional 3 residues in the domain I/II hinge area of DENV3 to DENV1 (Q52N, L53P and T55V) resulting in a total of 38 residues transplanted into DENV3. The viruses were designed to gain DENV1 1F4 and 14c10 hmAb neutralizing epitopes, while differentially preserving the DENV3-specific hmAb 5J7 neutralizing epitope, allowing us to measure loss of neutralization with the new panel of DENV3 hmAb. As a result of the quadripartite infectious clone design, all changes were isolated to the A and B fragments of the DENV3 genome backbone. cDNAs encoding E proteins incorporating three increasing sizes of the DENV1 ED1/EDIII transplant were synthesized (BioBasic, Buffalo, N.Y.) and incorporated into three different DENV3 fully assembled DNA genomes and transcribed. Then, the genome-length RNAs were electroporated into Vero-81 cells to generate a panel of viable recombinant rDENV3/1 viruses. Recombinant viruses were subjected to full-length sequencing to demonstrate the presence of appropriate subsets of mutations, as previously described (Gallichotte et al., 2018c; Gallichotte et al., 2017).

Two gain-of-function DENV1/3 recombinant chimeras were synthetically reconstructed. All of the varying surface residues in the ED1 of the DENV1 is were replaced with corresponding residues from DENV3 (DENV1/3 ED1-A). In parallel, varying residues in the surface and interior of the ED1 of DENV1 were replaced with DENV3 residues (rDENV1/3 EDI-B) in a second chimera that was constructed. Both viruses were viable and sequenced confirmed, allowing for systematic measures of gain-of-function neutralization assays with the new panel of DENV hmAb.

Generation of DENV3 genotype III/IV domain exchange virus panel. Recombinant DENV3 G-IV viruses encoding the G-III EDI, EDII or EDIII natural variation were recovered using reverse genetics Recombinant DENV3 G-III viruses encoding the G-IV EDI, G-IV EDII, or G-IV EDIII were isolated using reverse genetics. Briefly, residues in EDI, EDII or EDIII in the DENV3 Puerto Rico G-IV molecular clone were substituted into Sri Lanka 89 G-III molecular clone or vice versa using the quadripartite system described above and electroporated into C6/36 or Vero cells. All six viruses were viable and sequence-confirmed to encode the appropriate ED specific natural variation from each genotype.

Vero cell titration and focus assays. For viral titrations, viral stocks were diluted 10-fold serially in Vero cell medium supplemented with 2% heat-inactivated fetal bovine serum (HI-PBS; Hyclone Defined) and 1× antibiotic. The inoculum was added to Vero-81 cells that were seeded into a 96-well plate (2×10⁴ cells/well) the previous day and incubated at 37° C. for 1 hour, then overlaid with overlay medium (Opti-MEM I Grand Island, N.Y., with 1% methyl cellulose and 2% heat-inactivated FBS). Viral foci were detected at 44 to 48 h after infection, following fixation/permeabilization with 10% buffered formalin/0.01% saponin using primary murine mAbs 2H2 and 4G2 and secondary horseradish peroxidase (HRP)-conjugated goat anti-mouse IgG (Sigma), followed by TrueBlue substrate (KPL). Number and size of foci were analyzed with a CTL Immunospot instrument.

Vero cell neutralization assays. Neutralization on Vero-81 cells has been described previously (Gallichotte et al., 2015). Briefly, monolayers of Vero-81 cells in 96-well plates were inoculated with a virus and antibody or serum mix that had been incubated for 1 h at 37° C. to allow for Ab:virion binding. Following a 1 hr incubation on cells at 37° C. for infection, cells and inoculum were overlaid with overlay medium (see above). Viral foci were detected at 44 to 48 h after infection, following fixation/permeabilization with 10% buffered formalin/0.01% saponin using primary mAbs 2H2 and 4G2 (Swanstrom et al., 2016) and secondary horseradish peroxidase (HRP)-conjugated goat anti-mouse IgG (Sigma), followed by TrueBlue substrate (KPL). Numbers of foci were analyzed with an Immunospot Analyzer instrument (Cellular Technology Limited). All hmAb neutralization assays were performed as eight-point dilution curves done in duplicate with at least 2 independent experiments. Variable slope sigmoidal dose-response curves are calculated with top or bottom restraints of 100 or 0, respectively. EC₅₀ is the concentration of antibody that neutralizes 50% of the virus being tested.

U937-DC-SIGN neutralization assay. The neutralizing potency of the hmAbs was measured using a flow cytometry-based neutralization assay with the U937 human monocytic cell line stably transfected with DC-SIGN(Kraus et al., 2007). At an initial concentration of 15,000 ng/mL, hmAbs were serially diluted 3-fold 12 times in RPMI supplemented with 2% FBS. A dilution of virus that infects between 8-15% of the U937 cells (previously determined by virus titration) was added to the hmAb dilutions and incubated for 1 hour at 37° C. Following incubation, the cells were centrifuged at 252×g for 5 minutes and resuspended in 100 μL RPMI medium. Next, cells were fixed in 4% paraformaldehyde, incubated for 10 minutes at room temperature, and centrifuged at 252×g for 5 min. Following this, cells were blocked in permeabilization buffer (0.1% saponin, 5% bovine serum albumin in 1× phosphate-buffered saline [PBS]) for 30 minutes at room temperature. Then, cells were incubated with anti-E mAb 4G2 conjugated to Alexa 488, diluted in blocking buffer (0.5% bovine serum albumin and 0.02% sodium azide in 1× PBS) for 25 minutes at room temperature. Finally, cells were washed and resuspended in PBS. Acquisition of the infected cells was performed with a Guava flow cytometer (EMD Millipore) by gating Alexa 488-positive cells. The data were analyzed using a nonlinear, 4-parameter dose-response regression analysis with Prism software (GraphPad). The NT₅₀ was determined as the concentration of the hmAb dilution that achieved a 50% reduction of the infection compared to infection control. Data generated had to meet the quality control criteria, whereby the sigmoidal dose-response regression fit had to include an absolute sum of squares of <0.2 and a coefficient of determination (R²) of >0.9.

Animal studies. This study was performed in strict accordance with the recommendations in the Guide for the Care and Use of Laboratory Animals of the National Institutes of Health. All procedures were approved by the U.C. Berkeley Animal Care and Use Committee guidelines. AG129 mice were bred in the Animal Facilities at U.C. Berkeley. Mice 6 to 8 weeks of age were administered 50 μg of one of the newly isolated DENV3-specific hmAbs, DENV3 hmAb 5J7, or an isotype control antibody (IgG1) intraperitoneally (i.p.) in a total volume of 200 μL, 24 h prior to DENV inoculation. A sublethal dose (5×10⁶ PFU) of rDENV3 strain UNC 3009, genotype III, was administered intravenously (i.v.) in a total volume of 100 μL. Seventy-two hours post-infection, mice were sacrificed, spleens were harvested and placed in Trizol, and total RNA was extracted. Viral RNA burden and GAPDH levels in spleen were assessed by quantitative RT-PCR. Virus load in genome equivalents (GE) was normalized by dividing by ug of glutaraldehyde 3-phosphate dehydrogenase (GAPDH).

Quantification and statistical analysis. Statistical analysis was performed using Prism 5.0 (GraphPad, La Jolla, Calif.). Variable slope sigmoidal dose-response curves are calculated with top or bottom restraints of 100 or 0, respectively. EC₅₀ is the concentration of antibody that neutralizes 50% of the virus being tested. Non-parametric Kruskal-Wallis test with Dunn's multiple comparisons was calculated with multiplicity adjusted P-values. P-values are indicated by * symbol in plots; **=p<0.005, ***=p<0.0005, ****=p<0.0001. Statistical details of experiments can be found in the figure legend for FIG. 7.

Data and code availability statement. The published article includes all datasets generated or analyzed during this study. This study did not generate code.

### Example 2

### Results

Isolation of TS hmAbs from children previously infected with DENV3. To define the antigenic landscape of DENV3, the inventor immortalized memory B cells from children with previous laboratory-confirmed DENV3 infection in a Nicaraguan cohort study. Peripheral blood mononuclear cells (PBMCs) were collected from one individual who had a primary DENV3 infection (followed by an inapparent DENV1 infection) and from two DENV2-immune individuals who experienced a secondary DENV3 infection (FIG. 1A, Table B). The inventor transformed B cells and isolated DENV3 hmAbs from these PBMC samples as described (Nivarthi et al., 2017; Smith et al., 2012) (FIG. 51, Tables B and C). The percentages of DENV E protein-reactive B cells in circulation were estimated as 1.8, 1.1 or 1.6% of transformable B cells for subjects 3243, 985, or 1791, respectively.

The hmAbs that bound only to DENV3 (and not to DENV1, 2 or 4) were evaluated for neutralization activity against serotypes DENV1-4 (FIG. 1B). Fifteen DENV3-specific neutralizing hmAbs were isolated from the PBMCs of the 3 subjects. DENV3 neutralizing antibody potencies varied, but included antibodies that were (a) equally potent as the previously described hmAb 5J7 (e.g., hmAbs DENV-297, -354, -406, and -415)(0.2-0.35 μg/ml EC₅₀), (b) more potent than hmAb 5J7 (e.g., hmAb DENV-115, -144, -286, -290, -298, -404, -419, -437, and -443)(<0.035 μg/ml EC₅₀), or (c) less potent than hmAb 5J7 (e.g., hmAbs DENV-66 and -236)(>0.2 μg/ml EC₅₀) (FIG. 1C). A low level of cross-neutralization against some strains of DENV1 was detected with hmAb DENV-144, derived from subject 3243, who had been sequentially infected with DENV3 followed by DENV1 (FIG. 1A, Table B). It is noteworthy that these two different DENV1 Westpac variants encode two amino acid differences in EDI (residues I161T, T293I), which may influence neutralization. To determine whether any of these hmAbs targeted the hmAb 5J7 quaternary epitope, each was tested for its ability to neutralize the DENV4/3 M16 recombinant virus, which presents the DENV3 hmAb 5J7 epitope in the structural context of a DENV4 E glycoprotein backbone (Widman et al., 2017). Although all newly generated hmAbs neutralized the parental DENV3, none neutralized the DENV4/3 M16 recombinant virus, indicating that the new DENV3 hmAbs do not recognize the hmAb 5J7 epitope (FIGS. 1D-E).

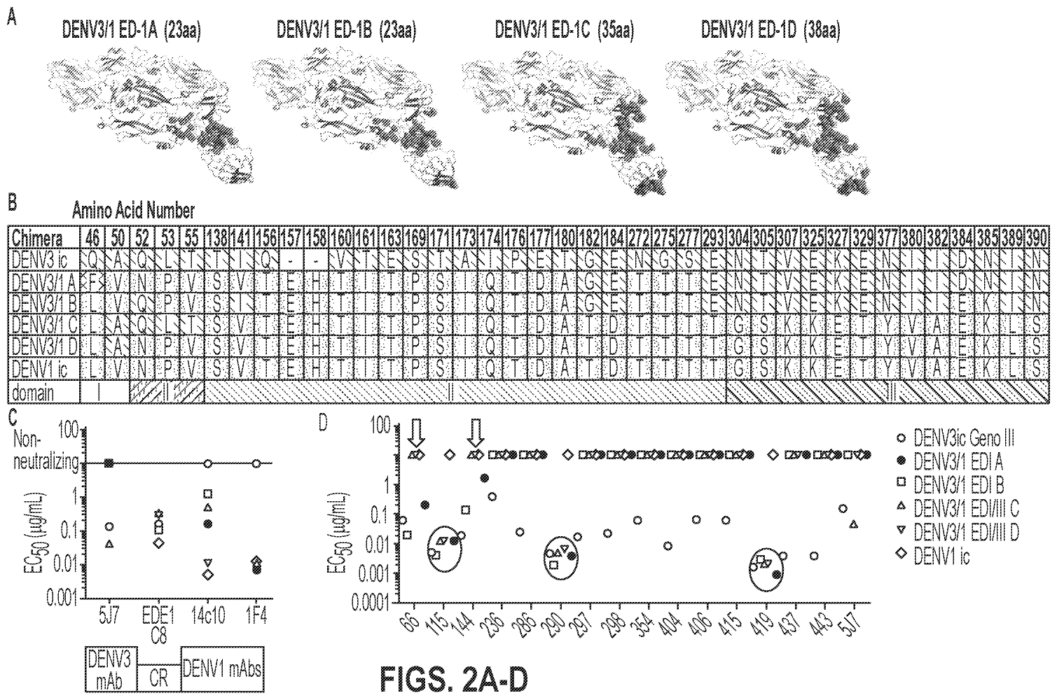

Epitope mapping using DENV3 loss-of-function recombinant viruses. Recombinant chimeric DENVs were used previously to map epitopes in DENV1, 2, 3 or 4 viruses recognized by murine or human mAbs (Gallichotte et al., 2018a; Gallichotte et al., 2018b; Gallichotte et al., 2018c; Gallichotte et al., 2017; Swanstrom et al., 2019). To identify the epitopes recognized by the 15 new DENV3 TS hmAbs, a panel of DENV3 loss-of-function (LOF) mutant viruses was generated. Starting with a previously described DENV3/1 EDI-A chimeric virus which incorporates 22 residues of the DENV1 EDI hmAb 1F4 footprint in the DENV3 E protein (Swanstrom et al., 2018), progressively larger portions of DENV1 TS hmAbs 1F4 and 14c10 antigenic region residues were introduced, designated DENV3/1 ED1-B, DENV3/1 EDI/III-C and DENV3/1 EDI/III-D (FIG. 2A, Table A-a). These epitope domains, which mostly reside in EDI and/or a portion of EDIII of DENV1, were transplanted into the DENV3 backbone (Messer et al., 2016). All recombinant viruses replicated efficiently in Vero cell monolayer cultures to titers of 10⁵−10⁶ FFU/mL (FIGS. 2A-C, FIGS. S2A-B).

To demonstrate that appropriate epitope exchange had been achieved in these chimeras, the ability of the DENV3 TS 5J7 hmAb and the DENV1 TS hmAbs 1F4 or 14c10 to neutralize the panel of wild-type or DENV3/1 chimeric viruses was investigated (FIG. 2C). Whereas the DENV1 TS hmAb 1F4 neutralized all 4 of the DENV3/1 chimeras, only the DENV3/1 EDI/III-D (38 amino acids) chimera fully restored the DENV1 14c10 antibody neutralization phenotype, reflecting transplantation of the entire 14c10 epitope, which extends across EDI, EDIII and the EDI/II hinge region (Teoh et al., 2012). The other three chimeras partially restored the 14c10 neutralization phenotype. Residues Q52N, L53P and T55V in the EDI/II hinge region were critical for 14c10 neutralization in DENV1 chimeras (FIG. 2C). Conversely, neutralization by hmAb 5J7 was retained in DENV3/1 EDI/III-C, but not in the DENV3/1 EDI/III-D recombinant virus, demonstrating the critical importance of these same three residues for hmAb 5J7 neutralization in DENV3 (FIG. 2C). These data also support atomic structures showing that the hmAb 5J7 and 14c10 epitopes extend in opposite directions from an area of overlap within the EDI/II hinge in their respective serotypes. Even though the mAb 1F4 epitope overlapped the EDI/II hinge area (Fibriansah et al., 2014), variation in this area did not hinder its ability to neutralize viruses in the chimeric panel. As a control, the recombinant form of the CR hmAb EDE1 epitope-specific hmAb C8 also neutralized the panel of chimeric viruses.

The ability of each newly generated DENV3 hmAb to neutralize viruses in the chimeric DENV3/1 virus panel was tested next. The DENV3-specific hmAbs grouped into three neutralization classes (FIG. 2D). Ten of the fifteen hmAbs (hmAbs DENV-236, -286, -297, -298, -354, -404, -406, -415, -437, or -443) did not neutralize the DENV3/1 chimeras, suggesting that loss of 23 core residues in the DENV3 EDI (that differ from DENV1 EDI) impacts binding and/or neutralization of this group (FIG. 3D). These 10 hmAbs, designated as Group 1 antibodies, likely target the core residues in the DENV3 EDI domain. The second cluster (designated group 2) includes three hmAbs that efficiently neutralized wild-type DENV3 virus and the entire panel of DENV3/1 loss-of-function chimeric viruses (hmAbs DENV-115, -290 and -419). As the constructs with the largest transplanted regions include DENV1 residues from EDI and EDIII but leave EDII as found in DENV3, the data suggested that hmAbs DENV-115, -290 and -419 target residues in EDII and perhaps a small portion of EDIII of DENV3. The third group (hmAbs DENV-66 and DENV-144) only neutralized the chimeras with the smallest transplanted regions, DENV3/1 EDI-A and DENV3/1 EDI-B, suggesting that DENV3 residues outside of the EDI domain, perhaps in EDIII and/or a smaller footprint in EDI, likely contribute to binding and neutralization of the group 3 antibodies. Groups 1 and 3 contain a mixture of weak to highly potent neutralizing antibodies, whereas all three group 2 antibodies exhibited high neutralizing potency.

Chimeric DENV1/3 gain-of-function (GOF) recombinant virus mapping. Chimeric loss-of-function E glycoproteins may disrupt long-range protein-protein interactions and complicate the interpretation of DENV3/1 antibody-epitope map locations. Therefore, a panel of DENV1/3 gain-of-function (GOF) EDI mutant viruses was designed and recovered (FIG. 3A) to validate the predicted EDI map locations of the 10 group 1 DENV3 hmAbs. Using a DENV1 molecular clone (Gallichotte et al., 2017), progressively larger portions of the EDI domain from DENV3 were introduced into DENV1 (FIG. 3). In the DENV1/3-EDI-A recombinant virus, DENV1 surface contact residues for hmAb 1F4 and 14c10 antibodies in EDI were replaced with the corresponding DENV3 residues. The virus expressed by this construct should not be neutralized by DENV1 TS hmAbs 1F4 or 14c10 nor the DENV3 TS mAb 5J7 (FIGS. 3A-C). In DENV1/3 EDI-B, the entire EDI of DENV3 was transplanted into DENV1, including both the previously described surface residues and interior residues (e.g., V141I, P169S, A180T, T182G, D184E and T293E). In addition, DENV1 residues E157 and H158 were deleted, because these two amino acids do not exist in the DENV3 EDI domain. This construct probes the role of both the surface and interior residues in hmAb binding and neutralization. The two GOF chimeric viruses replicated efficiently in Vero cell culture monolayers to titers of ˜10⁵ FFU/mL (FIG. 3A-D, FIGS. S2-S3).

Consistent with the defined structural interaction domains of each antibody/epitope pair, both DENV1/3 EDI-A and DENV1/3 EDI-B chimeric viruses were not neutralized by the DENV1 TS hmAbs 1F4, 14c10 nor the DENV3 TS hmAb 5J7 (FIG. 3D). Upon testing the new panel of DENV1/3 EDI GOF chimeras against the ten group 1 DENV3 hmAbs, nine mAbs efficiently neutralized both DENV1/3 chimeric viruses, but not DENV1. The DENV1/3 EDI-B chimera was neutralized more efficiently than the DENV 1/3 EDI-A chimera by seven of the nine hmAbs with similar neutralization potency compared to the DENV3 parental virus (e.g., group 1a EDI hmAbs DENV-286, -298, 354, -404, -406, -437 and -443) (FIG. 3D). Unexpectedly, two antibodies (group 1b EDI hmAbs DENV-236 and -297) neutralized both DENV1/3 EDI chimeras more efficiently than the parental DENV3 virus. These data suggest that the DENV3 group 1 hmAbs engage the DENV3 EDI region using at least two different binding patterns that appear to be dictated by at least in part, interior residues (e.g., V141I, P169S, A180T, T182G, D184E and T293E) in EDI of the chimeric viruses. A third binding pattern (group 1c) is represented by the group 1 antibody hmAb DENV-415, which did not neutralize either of the DENV1/3 EDI GOF chimeras. The DENV1/3 EDI-A or DENV1/3 EDI-B chimeras did not alter the group 2 antibody neutralization titers. In contrast, the group 3 antibodies had distinct patterns, with hmAb144 (group 3b), but not hmAb66 (group 3a), neutralizing DENV1/3 EDI-A and DENV1/3 EDI-B.

To determine if the DENV3-neutralizing hmAbs bound to epitopes mainly contained within a single protomer or epitopes that span the two protomers forming the E homodimer, the hmAbs were tested for binding to DENV3 recombinant E (recE) monomer or stabilized recE dimers (FIG. S4). Epitopes contained within a single E monomer or domain should be efficiently presented on recE monomers, whereas hmAbs with epitopes that spread across the dimer interface should bind preferentially to recE dimers (Dejnirattisai et al., 2015b; Metz et al., 2017). Indeed, hmAb DENV-415 did not bind to recE monomers but did show weak binding to recE dimers, suggesting it recognizes a quaternary epitope, perhaps requiring residues in neighboring E proteins in addition to EDI. Only group 1b hmAbs DENV-236 and -297 showed no preference for recE dimers over monomers, suggesting that they recognize epitopes in the EDI of a single E protomer, whereas the remaining 12 hmAbs recognize quaternary epitopes that span the E homodimer similar to DENV-415.

Together, these data suggest the presence of new functional neutralization epitopes on the surface of DENV3 E protein. Group 1 hmAbs DENV-236, -286, -297, -298, -354, -404, -406, -437 and -443, and to a lesser extent DENV-415, are predicted to recognize epitopes in EDI in at least 3 unique, yet perhaps overlapping, patterns from the previously described DENV3 neutralizing antibody, 5J7. Group 2 (e.g., hmAbs DENV-115, -290 and -419) likely map in EDII. The group 3a DENV3 TS hmAb DENV-66 did not neutralize any of the DENV1/3 EDI GOF chimeric viruses, suggesting that its epitope either overlaps partially with or resides outside of DENV3 EDI, perhaps in EDIII. Surprisingly, the group 3b hmAb DENV-144 could neutralize both DENV1/3 ED-I GOF chimeras (FIG. 3D) and the DENV3/1 EDI LOF chimeras (FIG. 2D). These data suggest the presence of another, likely complex, binding interface that depends on residues in DENV3 EDI and EDIII. However, it seems likely that the hmAb DENV-144 epitope site in EDI must be generally resistant to the incorporation of extensive DENV1 variation across the region. This hypothesis is also consistent with DENV-144 low-level cross neutralization of the Nauru Westpac 1974 strain that encodes variation in EDI.

HmAb neutralization phenotypes for viruses of DENV3 genotypes I to IV. To validate the location of the DENV3 epitopes, natural variation encoded within a panel of recombinant DENV3 viruses representing genotypic variation in field strains was tested to determine if this variability altered the neutralization profiles of hmAbs in the panel (FIGS. 4A-4B). Viruses in the DENV3 recombinant genotype panel encode the E glycoproteins from genotypes I, II, III or IV (G-I to -IV) introduced into the DENV3 Sri Lanka G-III backbone (Messer et al., 2012). Although each genotype strain encodes distinct amino acid differences across EDI, EDII and EDIII, all DENV3 genotypes were highly sensitive to neutralization by hmAb 5J7. All group 1a EDI hmAbs neutralized viruses from all 4 genotypes. In contrast, group 1b hmAbs DENV-236 and DENV-297 did not neutralize the G-IV virus, which encoded 7 unique amino acid substitutions in EDI (FIG. 4C). These data further suggest group 1b hmAbs DENV-236 and DENV-297 may use a different set of interaction residues in EDI as compared with group 1a EDI hmAbs DENV-286, -298, -354, -404, -406,-437 and -443. Of note, group 1c hmAb DENV-415 neutralized all of the viruses in the DENV3 genotype panel, although the half-maximal effective concentration (EC50) values for neutralization varied by ˜10-fold. G-III viruses were neutralized efficiently, whereas G-I, -II and -IV viruses were more resistant. Among the group 2 antibodies that may target EDII, hmAb DENV-290 efficiently neutralized viruses from all of the genotypes, whereas hmAbs DENV-115 and DENV-419 did not neutralize virus from G-IV (FIG. 4C). G-IV contains nine unique amino acids substitutions in EDII (FIGS. 4A-B). Since hmAbs DENV-115, -290 and -419 neutralized all of the EDI/EDIII-exchanged DENV3/1 and DENV1/3 chimeras, these data support their recognition of at least two unique and/or partially overlapping epitopes in EDII. Group 3 antibodies also demonstrated disparate neutralization phenotypes, since group 3b hmAb DENV-144 neutralized viruses from all four genotypes, whereas group 3a hmAb DENV-66 neutralized only G-III strains. As EDIII contains amino acid substitutions in G-I, -II and -IV as compared to G-III, these data support mapping studies that DENV-66 recognizes EDIII (FIG. 4C). For the hmAb DENV-144 epitope, areas that were not excluded by the DENV3/1 chimera panel and were conserved among the 4 genotypes were identified. Recognizing DENV-144 is the most complex functional epitope to define, these data suggest that hmAb DENV-144 recognizes EDI perhaps at the ED I/III interface. Thus, these studies define the core domains/sequences that are required for the functional neutralizing activities of each antibody in the E glycoprotein.

Fine mapping of epitopes using genotype differences. As natural variation in DENV3 G-IV contains clustered variation in EDI, II and III that altered the neutralization profiles of group 1-3 hmAbs, exchange of the ED regions between susceptible (e.g., G-III) and resistant (e.g., G-IV) genotypes of DENV3 should localize the epitope domain of selected group 1, 2 and 3 hmAbs. Reverse genetics was used to introduce either the EDI, II, or III regions from the resistant DENV3 G-IV E glycoprotein into the sensitive DENV3 G-III strain (FIGS. 4D-E, FIGS. S5A-B) or vice versa, allowing us to map critical functional residues using both GOF and LOF studies. Focusing first on the group 1b antibodies that efficiently neutralized DENV3 G-III but not G-IV viruses, mAbs DENV-236 and -297 were observed to gain neutralizing activity against DENV3 G-IV mutant viruses that encode the EDI, but not the EDII or EDIII domains from G-III (FIG. 4D). Additionally, hmAbs DENV-236 and -297 lost neutralizing activity against the sensitive DENV3 G-III mutant virus when EDI was replaced with G-IV EDI, but not EDII or EDIII (FIG. 4E). Therefore, the epitopes for group 1b hmAbs DENV-236 and -297 should reside in EDI. Group 1c hmAb DENV-415, which did not gain neutralizing activity against the DENV3/1 chimeras, had neutralization profiles that shifted nearly 10-fold when the G-III EDIII was present in this set of DENV3 genotype mutant viruses. In fact, hmAb DENV-415 gained potency (24-fold) in neutralization of the DENV3 G-IV mutant when EDIII was converted to G-III, whereas converting EDI or EDII variation did not affect neutralization. Conversely, when EDIII of DENV3 G-III was replaced by EDIII of G-IV, 10-fold loss of neutralization potency occurred. These data suggest that the hmAb DENV-415 epitope region likely encompasses residues spanning across EDI-III, more so than the epitope targeted by other group 1 antibodies.

The group 2 hmAbs DENV-115 and -419 demonstrated a gain-of-neutralization potential in the resistant DENV3 G-IV mutants when residues in EDII, but not EDI or EDIII, were converted to G-III residues (FIG. 4D). Reciprocally, in the fully susceptible DENV3 III genetic backbone, neutralizing activity of these mAbs was lost when residues in EDII, but not EDI or EDIII, were changed to those in the DENV3 G-IV (FIG. 4E). Of note, when the EDIII of DENV3 G-III was inserted into the resistant DENV3 G-IV backbone, hmAb DENV-115 gained the ability to neutralize this virus weakly, albeit >1,000-fold less potently than fully susceptible strains. These data further suggest that the epitope for hmAb DENV-115, but not DENV-419, is distinct and may extend from EDII into EDIII. Nonetheless, these data identify critical portions of the epitopes for DENV-115 and -419 in EDII. Finally, the group 3 mAb DENV-66 neutralized the resistant DENV3 G-IV backbone only when the variation in EDIII, but not EDI or EDII, was changed to residues in G-III. Similarly, hmAb DENV-66 failed to neutralize the susceptible DENV3 G-III virus when its EDIII was converted to that of G-IV EDIII. These results confirm the assignment of the epitope for hmAb DENV-66 in EDIII (FIGS. 4D-E).

In vivo protection studies. The in vitro neutralization potency of anti-flavivirus antibodies does not always correlate with in vivo activity, especially when antibodies target different epitopes or have distinct mechanisms of action (Mukherjee et al., 2014; Pierson and Diamond, 2015). The interferon α/β and γ receptor-deficient AG129 mouse strain is an established model for DENV replication and pathogenesis (Balsitis et al., 2010; Johnson and Roehrig, 1999; Messer et al., 2016; Raekiansyah et al., 2005; Sarathy et al., 2018; Shresta et al., 2004; Shresta et al., 2006). Using a DENV3 replication model, representative hmAbs from different individuals that targeted each of the four distinct interaction sites across EDI, II and III were evaluated for their ability to reduce viral load in the spleens of mice when administered prophylactically prior to inoculation with a wild-type DENV3 virus (DENV3 UNC3009, G-III) (FIG. 5). The representative group 1a hmAbs DENV-298, -404, and -443 along with group 1c DENV-144, which recognize EDI and were isolated from three different individuals, showed mixed levels of capacity to reduce virus replication in vivo. The hmAbs DENV-298, -404 and -144 reduced virus titers by ˜10-fold as compared with hmAb DENV-443, which reduced infection by >3 log₁₀ genome equivalents (GE)/□g of GAPDH, below the limit of detection. Even 20 μg of hmAb DENV-443 reduced viral titers by 100-fold. These data were somewhat unexpected, as the in vitro neutralization potency of each group 1a hmAb in Vero cell culture was similar (e.g., IC₅₀ values: DENV-443=4-7 ng/mL, DENV-404=8-18 ng/mL, DENV-298=15-23 ng/mL). The group 2 EDII hmAbs DENV-115, -290 and -419, isolated from three different individuals who either experienced DENV3 as a primary or secondary infection, reduced virus titers by over ˜2 to >3 log₁₀ GE/μg of GAPDH. These DENV3 TS hmAbs also had high neutralizing potency in vitro (exhibiting IC₅₀ values of 4-5 ng/mL), suggesting that EDII is an important site for antibody neutralization and antiviral activity in mice. In contrast, the group 3 EDIII hmAb DENV-66 reduced virus titers by <10-fold in the spleen. For comparison, the previously described DENV3 neutralizing hmAb 5J7 reduced virus titers by an average of 1 log₁₀ as compared to the isotype control. Together, these data indicate that the new hmAbs identified following DENV3 infections in children from Nicaragua are among the most potently neutralizing DENV3 hmAbs isolated to date, both in vitro and in vivo, and principally target diverse epitopes in EDI and EDII, and to a lesser extent in EDIII on DENV3.

Sequence analysis of hmAb variable region genes. It is interesting that the group 1 EDI antibodies fall into three categories of binding interfaces as defined by LOF, GOF and genotype-exchange studies using neutralization assays. Surprisingly, sequence analysis revealed that the group 1b hmAb DENV-236 and -297 have similar heavy chains, even though they originated in two separate individuals. Moreover, these mAbs are also the only two DENV3 hmAbs that neutralized the chimeric DENV1/3 EDI recombinant viruses significantly better than they did the parental DENV3 viruses (FIGS. 3A-D). These mAbs are also the only two hmAbs in the panel that bound equally well to monomeric or dimeric recE and showed an inability to neutralize DENV3 G-IV (FIG. 4C). These shared characteristics may be a result of their similar heavy chains.

### Example 3

### Discussion

Dengue vaccine-induced immunity relies on the development and maintenance of long-term protective antibody titers, and B and T cell memory responses. A tetravalent live attenuated dengue vaccine (Dengvaxia) was poorly efficacious in DENV-naive individuals compared to DENV pre-immune individuals who received the vaccine (Halstead, 2017, 2018a, b) (Hadinegoro NEJM 2015; Sridar NEJM 2018). The best studied correlate of protective immunity after DENV infection is the development of high titers of serum neutralizing antibodies (Katzelnick et al., 2016). Moreover, recent studies with people exposed to natural DENV infections or live attenuated vaccines indicate that the specificity (TS/epitope specificity) rather than total quantity of neutralizing antibodies correlates best with long-term protection (Gallichotte et al., 2018b; Henein et al., 2017; Moodie et al., 2018; Sridhar et al., 2018). Indeed, DENV4 genotype variation in the E glycoprotein strongly correlated with reduced neutralization titers after vaccination (Gallichotte et al., 2018b). These and other studies demonstrate a pressing need to identify improved correlates of antibody-mediated protective immunity. Here, the inventor isolated 15 DENV3 TS neutralizing hmAbs from three Nicaraguan children who experienced DENV3 primary or secondary infection. Using both GOF- and LOF-epitope chimeric viruses, three classes of neutralizing antibodies that likely reflect six functional neutralizing antigenic sites in and across EDI, II and III of DENV3 (FIGS. 6A-B) were identified. All but two hmAbs in group 1b bound to stabilized dimers of DENV3 E protein better than to monomeric recE, supporting the hypothesis that most neutralizing anti-DENV hmAbs recognize quaternary epitopes (Magnani et al., 2017; Metz et al., 2017; Rouvinski et al., 2017). Importantly, these data suggest that the neutralizing antigenic repertoire of DENV3, and potentially other DENV serotypes, is more complex than previously recognized.

Most of the durable human serum neutralizing response after primary infection is associated with TS antibodies that recognize a few well-defined epitopes centered within and spanning domains on the DENV E glycoprotein (de Alwis et al., 2012; Fibriansah et al., 2015b; Gallichotte et al., 2018a; Teoh et al., 2012). While this report has identified 15 new hmAbs that map within and/or span DENV3 EDI, II and III, other less well characterized DENV3 hmAbs (DV74.4 and DV7.9.3) are predicted to bind in EDI/II (Beltramello et al., 2010). Another TS hmAb (P3D05) isolated after tetravalent live attenuated vaccination remains unmapped (Magnani et al., 2017). The panels of hmAbs and chimeric viruses reported here will provide a powerful resource to determine if uncharacterized mAbs recognized known or unique epitopes in DENV3. In contrast to murine DENV3 TS Abs, which principally target EDIII, the studies performed with the inventor's new antibodies described here support earlier work showing that epitopes entirely contained within EDIII of DENV are infrequently targeted by strongly neutralizing human antibodies (Brien et al., 2010; Wahala et al., 2010).

Many groups have focused on defining the functional and structural properties of DENV CR antibodies elicited in adults after secondary infection (Dejnirattisai et al., 2015b; Li et al., 2018). However, the hmAbs elicited in pediatric subjects from DENV-endemic regions represent an understudied population. As the immune systems of adults and young children may differ in their capacity to recognize the number, location and complexity of DENV3 neutralizing epitopes in the E glycoprotein (Simon et al., 2015), defined analyses of the antibody repertoire in at-risk pediatric populations is important for evaluating vaccine performance In subject 3243, who experienced a primary DENV3 infection followed by an inapparent secondary DENV1 infection, DENV3-specific neutralizing hmAbs (DENV-66, -115 and -144) targeted three distinct areas of the E glycoprotein. Whereas the epitope targeted by hmAb DENV-144 is complex and predicted to extend across a portion of EDI into EDIII (FIGS. 6A-B), a combination of mapping techniques, coupled with its ability to bind dimeric but not monomeric E protein, suggests that hmAb DENV-115 recognizes a separate epitope in EDII that spans multiple protomers. Using similar approaches, hmAb DENV-66 was shown likely to target EDIII, analogous to DENV3 lateral-ridge epitope seen with murine mAbs (Brien et al., 2010). Thus, three spatially distinct DENV3-specific neutralizing epitopes in EDII and III induced by primary DENV3 infection were identified in PBMCs collected after a secondary DENV1 infection. Unlike murine mAbs, the EDII-specific hmAb DENV-115, but not the EDIII-specific hmAb DENV-66, substantially reduced DENV3 viral load in the spleen in vivo.

The majority of the inventor's hmAbs were isolated from two children who experienced DENV3 secondary infections several years after a primary DENV2 infection. Importantly, these two DENV3 secondary infections also induced antibodies that targeted EDII and EDI, which demonstrates the importance of these antigenic sites in type-specific DENV3 immunity. Together, these data support the idea that patient-specific polyclonal responses may target distinct neutralizing epitopes after infection. In these patients, ten of the twelve neutralizing hmAbs target EDI of the DENV3 E glycoprotein, and two of the hmAbs target EDII. This EDI epitope skewing was unexpected, given the expansive repertoire of epitopes targeted by antibodies from the first individual. DENV polyclonal neutralizing responses appear to target either an EDII/EDIII or an EDI epitope after a DENV2 primary infection (Gallichotte et al., 2018c) or a EDI/II hinge epitope following primary DENV1 and DENV3 infections (Andrade et al., 2017; Andrade et al., 2019). Using recombinant viruses and sera from a larger Nicaraguan pediatric cohort, substantial individual variation was noted in the proportion of DENV3 type-specific neutralizing antibody titers attributed to the 5J7 epitope (range, 0 to 100%), which further supports the notion of individual variation in epitope targeting (Andrade et al., 2017). This finding likely reflects inter-host immune variation associated with infection and depth of epitope mapping analyses. In the children experiencing DENV2 primary infections, it is also speculative to consider that the antibody variable genes in pre-existing EDI DENV2-reactive B cells mutated during the DENV3 infection to convert clones from DENV2-reactive to DENV3-specific, leading to epitope skewing of the response Immune responses between closely related flaviviruses are complex and may be impacted by pre-exposure histories that shape the antibody response to new strains or related viruses, as has been shown when Zika virus spread into DENV-endemic areas (Bhaumik et al., 2018; Grifoni et al., 2017; Stettler et al., 2016). Unfortunately, insufficient quantities of PBMCs prevented us from evaluating the antibody lineages identified in these children. Moreover, it is unclear if the new DENV3 neutralizing sites are also targeted following DENV3 vaccination. In tetravalent vaccinated macaques, hmAbs targeted multiple TS epitopes in EDI and EDII of DENV4 or the EDIII regions of some other serotypes, but not for DENV3 (Li et al., 2019). In human tetravalent vaccine recipients, both TS and CR neutralizing antibodies have been reported for DENV3, although it remains unclear whether these antibodies map to the same or different epitopes as those reported here after DENV3 infection (Magnani et al., 2017; Smith et al., 2013; Swanstrom et al., 2019).

Chimeric recombinant viruses provide a useful strategy for mapping epitope specific responses of monoclonal or polyclonal antibodies and may reveal novel interaction patterns within an ED (Gallichotte et al., 2018c; Gallichotte et al., 2017; Gallichotte et al., 2015; Widman et al., 2017). For example, the group 1a hmAbs DENV-286, -404, and -437 showed a 10-fold increase in their neutralization potency when the entire EDI from DENV3 was present. These data suggest that the surface topology of chimeric E glycoproteins is more authentic to wildtype virus when both surface and underlying residues are exchanged between serotypes. Two antibodies, hmAbs DENV-236 and -297, neutralized chimeric DENV1 viruses encoding the EDI of DENV3 more potently than wild-type DENV3 virus. These group 1b antibodies also are highly susceptible to natural variation in DENV3 genotypes, suggesting that the group 1a and 1b antibodies engage EDI in at least two different patterns. The group 1c hmAb DENV-415 epitope placement is more difficult to ascertain from these studies, as the epitope likely overlaps with that of other group 1 antibodies but represents a third binding modality since it gained neutralization to DENV3 G-IV with G-III ED3. The epitope for group 3b hmAb DENV-144 also overlaps with those of group 1 hmAbs, since it gained neutralization to the DENV1/3 chimeras that had EDI from DENV3. The mechanism of DENV-144 cross neutralization of the Nauru Westpac DENV1 strain remains to be investigated but may reflect EDI microvariation that directly or indirectly impacts hmAb function; similar antibodies have been described with ZIKV (Robbiani et al., 2017; Zhao et al., 2020). The hmAbs DENV-115, -290 and -419 target EDII. For other DENV serotypes and the related flavivirus ZIKV, EDII, and perhaps EDIII, is an important target for human neutralizing antibodies (Collins et al., 2019; Gallichotte et al., 2019; Hasan et al., 2017; Long et al., 2019; Sapparapu et al., 2016; Wang et al., 2017; Zhao et al. , 2020). The group II EDII antibodies the inventor reports here are among the most potent TS DENV3 neutralizing antibodies identified in humans. Studies with recombinant E dimers suggest that the group II hmAbs recognize a quaternary epitope that spans EDII regions of two protomers, however, they could also conceivably span the EDII regions encoded within two adjacent dimers on an E glycoprotein raft. As natural variation modulates the performance of these antibodies, they may target two sites in EDII. Clearly, atomic resolution structures of antibody-antigen complexes are needed to resolve the epitope specificities and interaction networks of each of these different hmAbs, perhaps revealing novel mechanisms of neutralization and targets for DENV3 protective immunity.

Consonant with the circulation of DENV3 G-III viruses throughout the Caribbean and Central America in 2004-11 (Gutierrez et al., 2011; OhAinle et al., 2011), all 15 hmAbs studied here efficiently neutralized DENV3 strains encoding a G-III E glycoprotein. However, natural variation altered the neutralization potency of a subset of antibodies targeting EDI (group 1b antibodies), EDII (hmAb 115 and hmAb 419) and EDIII (hmAb 66) (FIG. 6A). DENV3 genotypes G-I, -II, -III and -V circulate currently, whereas G-IV is comprised by an ancestral lineage from the Caribbean (Puerto Rico:1963/77) that is now extremely rare in human populations (King et al., 2008; Waman et al., 2017). The DENV3 genotype panel used here includes E glycoproteins from G-I through -IV (Messer et al., 2012) and is missing some more recently reported variation in this serotype. Historically, natural variation is thought to play a limited role in antigenic variation and DENV immunologic escape from pre-existing immunity (Holmes and Twiddy, 2003). However, studies have demonstrated as much as 10- to 15-fold differences in DENV3 neutralization phenotypes across genotypes, using monoclonal and polyclonal antibodies collected following primary DENV3 infections (Messer et al., 2012; Sukupolvi-Petty et al., 2013; Wahala et al., 2010). Although speculative, these data suggest that DENV3 genotypic variation might contribute to breakthrough infections in rare individuals, especially those who developed limited polyclonal serum antibody responses that target one or a limited subset of neutralizing epitopes.

The in vivo potencies of the EDI antibodies were highly variable, and the panel of recovered mAbs included both potent and weak inhibitors of virus replication in mice, as seen with the group 1a hmAbs DENV-443 and -298, respectively. This phenotype may reflect subtle differences in epitope targeting and neutralization potency within EDI, an impact on antibody performance as a function of maturation status, or alternatively may reflect inherent differences in Fc effector functions encoded by these antibodies (Lee et al., 2013; Lofano et al., 2018). All three EDII antibodies tested potently reduced virus load in vivo, with DENV-290 being the most effective, suggesting the importance of EDII in protective immunity. Future studies are planned to evaluate the potency of a subset of these neutralizing human antibodies in a lethal DENV3 challenge mouse model under prophylactic and therapeutic conditions.

The hmAbs, recombinant proteins and chimeric viruses will serve as key reagents for evaluating vaccine immunogenicity and for measuring epitope- and ED-specific responses associated with natural infections and/or vaccinations. As DENV-naïve children receiving the Dengvaxia tetravalent vaccine are at increased risk for severe DENV after infection (Ferguson et al., 2016), and TAK-003 showed reduced efficacy in seronegative populations against DENV3 (Biswal et al., 2019), there remains a critical need for better correlates of protective immunity and improved vaccines in children. The study demonstrates the importance of evaluating the TS neutralizing antibody responses in children experiencing primary or secondary infections with DENV. The DENV3 antibody neutralizing landscape is complex, with antibodies falling into multiple groups as described here (FIGS. 6A-B) and previously (Fibriansah et al., 2015b; Widman et al., 2017). As the most potent DENV3-specific hmAbs target EDI and ED II in vivo, it is possible that variation in the potency and epitope specifies of individual host responses after DENV3 infections or vaccination may result in complex patterns of neutralizing antibodies in polyclonal sera. Variation in the serological repertoire may also correlate with protective immunity or susceptibility to repeat infection by the same or different DENV3 genotypes (Waggoner et al., 2016). The complexity of the DENV3 neutralizing antigenic landscape suggests that the diversity of neutralizing epitopes in other DENV strains also remains largely undiscovered. Given the global health crisis associated with the hundreds of millions of DENV infections worldwide and the issues surrounding tetravalent vaccine outcomes, analysis of the antibody repertoire and epitope specificities elicited after vaccination may well determine efficacy and the likelihood of breakthrough infections leading to more severe disease in children and adults.

TABLE A-a
Dengue chimeric viruses

| | RECOMBINANT VIRUS | DENV3 517 | DENV1 1F4 | AA | EPITOPE | NEUTRALIZED |
|---|---|---|---|---|---|---|
| NAME | BACKBONE | NEUT | NEUT | CHANGES | TRANSPLANT | BY HMAB |
| DENV4/3 | DENV4 Baric genotype I | +++ | − | 36 aa | 517 DENV3 | 517 |
| M16 | | | | | | |
| DENV3/1 | DENV3 Baric genotype III | − | +++ | 23 aa | 1F4 DENV1 | 66, 115, 144, |
| EDI A | | | | | | 290, 419 |
| DENV3/1 | DENV3 Baric genotype III | − | +++ | 23 aa | IF4 + 14c10 | 66, 115, 144, |
| EDI B | | | | | DENV1 | 290, 419 |
| DENV3/1 | DENV3 Baric genotype III | +++ | +++ | 35 aa | IF4 + 14c10 | 115, 290, |
| EDI/III C | | | | | DENV1 | 419, 517 |
| DENV3/1 | DENV3 Baric genotype III | − | +++ | 37 aa | IF4 + 14c10 | 115, 290, |
| EDI/III D | | | | | DENV1 | 419 |
| DENV1/3 | DENV1 Baric | − | − | 18 aa | EDI surface aa | 236, 286, 297, 298, |
| EDI A | | | | | DENV3 | 354, 404, 406, 437, |
| | | | | | | 443, 144 |
| DENV1/3 | DENV1 Baric | − | − | 22 aa | EDI all aa | 236, 286, 297, 298, |
| EDI B | | | | | DENV3 | 354, 404, 406, 437, |
| | | | | | | 443, 144 |

TABLE A-b
Dengue chimeric viruses

| | | Domain | AA | DENV- | DENV- | DENV- | DENV- | DENV- | DENV- |
|---|---|---|---|---|---|---|---|---|---|
| Name | Recombinant Virus Backbone | Swap | changes | 236 | 297 | 415 | 115 | 419 | 66 |
| DENV3 G-III | DENV3 baric genotype III | None | 0 | *** | *** | *** | *** | *** | *** |
| | Sri Lanka | | | | | | | | |
| DENV3 G-IV | DENV3 baric genotype IV | None | 0 | − | − | * | − | − | − |
| | Puerto Rico | | | | | | | | |
| DENV3 GIV with | DENV3 baric genotype IV | EDI | 9 aa | +++ | +++ | + | − | − | − |
| G-III EDI | Puerto Rico | | | | | | | | |
| DENV3 GIV with | DENV3 baric genotype IV | EDII | 9 aa | − | − | + | +++ | +++ | − |
| G-III EDII | Puerto Rico | | | | | | | | |
| DENV3 GIV with | DENV3 baric genotype IV | EDIII | 6 aa | − | − | +++ | + | − | +++ |
| G-III EDIII | Puerto Rico | | | | | | | | |
| DENV3 GIII with | DENV3 baric genotype III | EDI | 8 aa | − | − | ++ | +++ | +++ | +++ |
| G-IV EDI | Sri Lanka | | | | | | | | |
| DENV3 GIII with | DENV3 baric genotype III | EDII | 10 aa | +++ | +++ | ++ | − | − | +++ |
| G-IV EDII | Sri Lanka | | | | | | | | |
| DENV3 GIII with | DENV3 baric genotype III | EDIII | 6 aa | +++ | +++ | +++ | +++ | +++ | − |
| G-IV EDIII | Sri Lanka | | | | | | | | |

TABLE C
Capture ELISA and FRNT data. Related to FIG. 1 and FIG. S1

| | | | | | | | | | NEUT-WUSTL | | | | | | | | | | | |
|---|---|---|---|---|---|---|---|---|---|---|---|---|---|---|---|---|---|---|---|---|
| | | | | | ELISA-VUMC | | | | (ng/ml) | | | | | | | | | | | |
| | | | | | (ng/ml) | | | | DV1 | | | | | | | | UNC-CH (ng/ml) | | | |
| | | | | | DV1 | DV2 | DV3 | DV4 | Nauru/ | DV2 | DV3 | DV4 | NEUT-UCB (ng/ml) | | | | | | DV3ic | |
| | | Sample | IgG | | Thailand/ | Thailand/ | Phillippines/ | Indonesia/ | West | Thailand/ | Thailand/ | Columbia/ | | | DV3 | | DV1ic | | SriLanka | DV4ic |
| mAb | Donor | Harvest | sub- | light | 16007/ | 16681/ | 16562/ | 1036/ | Pac/ | 516803/ | CH53489/ | TVP-376/ | DV1 | DV2 | N2845- | DV4 | West | DV2ic | 89 Geno | SriLanka |
| Clone | ID | date | class | chain | 1964 | 1964 | 1964 | 1976 | 1974 | 1974 | 1973 | 1982 | 1265-4 | N172-06 | 09 | N703-99 | Pac 74 | 16803 | III | 92 |
| 66 | 1037 | 2013 | IgG1 | κ | NB | NB | 63.2 | NB | NN | NN | NN | NN | NN | NN | 146.7 | NN | NN | NN | 66.2 | NN |
| 115 | 1037 | 2013 | IgG1 | λ | NB | NB | 106.9 | NB | NN | NN | 5.5 | NN | NN | NN | 87.0 | NN | NN | NN | 5.5 | NN |
| 144 | 1037 | 2013 | IgG1 | κ | NB | NB | 109.1 | NB | 329 | NN | 22.0 | NN | 2951 | NN | 39.4 | NN | NN | NN | 18.9 | NN |
| 236 | 985 | 2011 | IgG1 | λ | NB | NB | 390.0 | NB | NN | NN | 9.3 | NN | NN | NN | 15.0 | NN | NN | NN | 390.8 | NN |
| 286 | 1791 | 2011 | IgG1 | κ | NB | NB | 286.0 | NB | NN | NN | 4.4 | NN | NN | NN | 18.0 | NN | NN | NN | 26.3 | NN |
| 290 | 1791 | 2011 | IgG1 | κ | NB | NB | 225.0 | NB | NN | NN | 79.0 | N/A | NN | NN | 74.0 | NN | NN | NN | 4.4 | NN |
| 297 | 1791 | 2011 | IgG1 | λ | NB | NB | 142.0 | NB | NN | NN | 5.3 | NN | NN | NN | 21.0 | NN | NN | NN | 18.2 | NN |
| 298 | 1791 | 2011 | IgG1 | κ | NB | NB | 124.0 | NB | NN | NN | 8.0 | N/A | NN | NN | 100.0 | NN | NN | NN | 22.7 | NN |
| 354 | 1791 | 2011 | IgG1 | κ | NB | NB | 91.0 | NB | NN | NN | 8.1 | N/A | NN | NN | 100.0 | NN | NN | NN | 57.6 | NN |
| 404 | 985 | 2011 | IgG1 | κ | NB | NB | 37.3 | NB | NN | NN | 5.0 | NN | NN | NN | 33.0 | NN | NN | NN | 7.9 | NN |
| 406 | 985 | 2011 | IgG1 | λ | NB | NB | 136.0 | NB | NN | NN | 64.0 | NN | NN | NN | 224.0 | NN | NN | NN | 68.2 | NN |
| 415 | 985 | 2011 | IgG1 | κ | NB | NB | 26.2 | NB | NN | NN | 731.0 | NN | N/A | N/A | N/A | N/A | NN | NN | 57.6 | NN |
| 419 | 985 | 2011 | IgG1 | κ | NB | NB | 44.4 | NB | N/A | N/A | N/A | N/A | NN | NN | 2.8 | NN | NN | NN | 1.8 | NN |
| 437 | 985 | 2011 | IgG1 | κ | NB | NB | 25.8 | NB | NN | NN | 9.0 | NN | NN | NN | 207.0 | NN | NN | NN | 3.9 | NN |
| 443 | 985 | 2011 | IgG3 | λ | NB | NB | 15.1 | NB | NN | NN | 11.0 | NN | NN | NN | 57.0 | NN | NN | NN | 4.2 | NN |

Live-virus capture ELISA EC₅₀ values were determined at Vanderbilt University Medical Center, Vero-81 cell FRNT EC₅₀ values for WHO prototype strains for DENV 1-4 were performed at Washington University in St. Louis, U937 neutralization EC₅₀ values were determined at U.C. Berkeley, and Vero-81 cell FRNT EC₅₀ value were determined at University of North Carolina-CH.

TABLE 1
NUCLEOTIDE SEQUENCES FOR ANTIBODY VARIABLE REGIONS

| | SEQ | | |
|---|---|---|---|
| Clone | ID NO: | Chain | Variable Sequence Region |
| DENV- | 1 | heavy | CAGGTGCAGCTGGTGCAGTCTGGGGCTGAGGTGAAGAAGCCTGGGGCCCCAGTGAAGGTCTCCTGCGAGGCCTCTGG |
| 115 | | | ATACACATTCACCGACTATTTTATACACTGGGTGCGACAGGCTCCTGGACAAGGACTTGAGTGGATGGGATGGATCA |
| | | | ACCCTATCAGTGGTGGCACAAACTATCACCCGAGATTTCATGGCGGGGTCACCATGACCAGGGACACCTCCATGAAA |
| | | | GTAGCCTACATGGAACTTAAGAGGCTGACATCTGACGACACGGCCGTGTATTTCTGTGCGAGAGGTCGAGATTTTAG |
| | | | GGGTGGTTATTCCCAACTTGACTATTGGGGCCAGGGAACCCTGGTCACCGTCTCCTCA |
| | 2 | light | CAGTCTGTGCTGACTCAGCCACCCTCAGCGTCTGGGACCCCCGGGCAGAGGGTCACCATCTCTTGTTCTGGAGGCAG |
| | | | CTCCAACATCGCAATTAATACTGTAAACTGGTATCAGCAGGTCCCAGGAACGGCCCCCAAACTCCTCATGTATAGTA |
| | | | ATAATCAGCGGCCCTCAGGGGTCCCCGACCGATTCTCTGGCTCCAAGTCTGGCACCTCAGCCTCCCTGGCCATCAGT |
| | | | GGGCTCCAGTCTGAGGATGAGGCTGATTATTACTGTGCAACATGGGATGACAGTCTGAAAGATGTGCTATTCGGCGG |
| | | | AGGGACCAAACTGACCGTCCTA |
| DENV- | 3 | heavy | CAGGTGCAGCTGCAGGAGTCGGGCCCAGGACTGGTGAAGCCTTCGGAGACCCTGTCCCTCACCTGCACTGTCTCTGG |
| 144 | | | TGCCTCCATCAGTTCTTACTCGTGGAGCTGGATCCGGCAGCCCGCCGGGAGGGGACTTGAGTGGCTTGGGCGTATCT |
| | | | ATCCCAGTGGGAACACCAACTACAGTCCCTCCCTCAAGAGTCGACTCACCATGTCACTAGACACATCCAAGAACCAG |
| | | | TTCTCCATGAAGCTGACCTCTGTGAGCGCCGCGGACACGGCCGTCTATTACTGTGCGAGAGATCGGGAGCAGTGGCC |
| | | | CTTGTATTATGGTATGGACGTCTGGGGCCAAGGGACCCTGGTCACCGTCTCCTCC |
| | 4 | light | GAAATTGTGTTGACACAGTCTCCAGCCATCCTGTCTTTGTCTCCAGGGGACAGAGCCACCCTCTCCTGCAGGGCCAG |
| | | | TCAGAGTGTTTTCACCTACTTAGCCTGGTACCAACATAAACCTGGCCAGGCTCCCAGGCTCCTCATCTATGATGCAT |
| | | | CCAACAGGGCCTCTGGCATCCCAGCCAGGTTCAGTGGCAGTGGGTCTGGGACAGACTTCACTCTCACCATCAGCAGC |
| | | | CTAGAGCCTGAAGATTTTGCAGTTTATTACTGTCAGCAGCGTACCAAGTGGCCCCTGGCTTTCGGCGGAGGGACCAA |
| | | | GGTGGAGATCAAG |
| DENV- | 5 | heavy | CAGGTGCAGCTGCAGGAGTCGGGCCCAGGACTGGTGAAGCCTTCGGAGACCCTGTCCCTCAGTTGCACTGTCTCTGG |
| 286 | | | TGGCTCCATCAGTCCTGACTACTGGAGCTGGATCCGGCAGCCCCCAGGGAAGGGACTGGAGTGGCTTGGGTACATCT |
| | | | ATTCTGCTGGGAGCACCAGCTACAACCCCTCCCTCAAGAGTCGAGTCACCATGTCAGTAGACACGTCCAAGAACCAG |
| | | | TTATCCCTGAAACTGACCTCTGTGACCGCTGCGGACACGGCCGTGTATTACTGTGCGAGGACGGCGGGGAGTTTTTG |
| | | | GAGTGGTCGAGGCTGGTTCGACCCCTGGGGCCAGGGAACCCTGGTCACCGTCTCCTCA |
| | 6 | light | GAAATTGTGTTGACGCAGTCTCCAGCCACCCTGTCTTTGTCTCCAGGGGAAAGAGTCACCCTCTCCTGCGGGGCCAG |
| | | | TCAGAGTGTTAGCAGCAGCCACTTAGCCTGGTACCAGCAGAAACCTGGCCTGGCGCCCAGGCTCCTCATCTATGATG |
| | | | CATCCAACAGGGCCACTGGCGTCCCAGACAGGTTCAGTGGCAGTGGGTCTGGGACAGACTTCACACTCACCATCAGC |
| | | | AGACTGGAGCCTGAAGATTTTGCAGTGTATTACTGTCAACAGTATGGTAGCCCGCAGTACACTTTTGGCCAGGGGAC |
| | | | CAAGCTGGAGATCAAACGAACTGTGGCTGCACCA |
| DENV- | 7 | heavy | CAGTGTCAGGTGGAGCTGGTGGAGTCTGGGGGCGACGTGGTCCAGCCTGGGAAGTCCCTGAGACTCTCCTGTGCGGC |
| -290 | | | CTCTGGATTCACCTTCACTAACTATGCTATGCACTGGCTCCGCCAGGCTCCAGGCAAGGGGCTGGAGTGGGTGGCAG |
| | | | TCATATCTTCTGATGTCAACGATAAATACTACGCAGACTCCGTGAAGGGCCGATTCACCATCTCCAGAGACAATTCC |
| | | | AAGAACACCCTGTATCTGCAAATGAACAGCCTGACACCTGAAGACACGGCTGTGTATTACTGTGCGAGAGAGCAAGC |
| | | | CGTGGGAACAAATCCGTGGGCCTTTGACTACTGGGGCCAGGGAACCCTGGTCACCGTCTCCTCA |
| | 8 | light | CACATTGTGATGACCCAGAGTCCACTCTCTCTGTCCGTCACCCCTGGACAGCCGGCCTCCATCTCCTGCAAGTCTAG |
| | | | TCAGATCTCCTCTTGGGGTAGTGATGGAAAGACCTATTTGTATTGGTACCTGCAGAAGCCAGGCCAGTCTCCACAGC |
| | | | TCCTAATCTATGAAGTTTCCAGCCGATTCTCTGGAGTGTCAGATAGGTTCAGTGGCAGCGGGTCAGGGACAGATTTC |
| | | | ACACTGAAAATCAGCCGGGTGCAGGCTGAGGATGTTGGACTTTATTACTGCATGCAAGGTTTACACCTTCCGCTCAC |
| | | | CTTCGGCCAAGGGACACGACTGGAGATTAAA |
| DENV- | 9 | heavy | GAGGTGCAGCTGTTGGAGTCTGGGGGAGGCTTGGTACAGCCTGGGGGGTCCCTAAGACTCTCCTGTGCAGCCTCTGG |
| 297 | | | ATTCACCTTTAACAACTCTGCCATGGGCAGTTATGCCATGATCTGGGTCCGCCAGGCTCCAGGGAAGGGGCTGGAGT |
| | | | GGGTCTCAACTATTACCGGTACTGGTCTTACCACATACTACGCAGACTCCGTGAAGGGCCGGTTCACCGTCTCCAGA |
| | | | GACAATTCCAGGAACACGCTGCATCTGCAAATGAACAGCCTGAGAGCCGAGGACACGGCCGTCTATTACTGTGCGAA |
| | | | ATGGAATATAATTACTATGGCCCCTTTTGATATCTGGGGCCAAGGGACATTGGTCACCGTCTCTTCA |
| | 10 | light | CAGACTGTGGTGACCCAGGAGCCATCGTTCTCAGTGTCCCCTGGAGGGACAGTCACACTCACTTGTGGCTTGACCTC |
| | | | TGGCTCAGTCTCTACTAGTTACTATACCAGCTGGTACCAGCAGACCCCAGGCCAGGCTCCACGCACGCTCATCTACA |
| | | | AGACAAACACTCGCTCTTCTGGGGTCCCTGATCGCTTCTCTGGCTCCATCGTTGGGAACAAAGCTGCCCTCACCATC |
| | | | ACGGGGGCCCAGCCAGATGATGAATCTGATTATTACTGTGTGCTGTATGTGGGTAGTGGCATTTGGGTGTTCGGCGG |
| | | | AGGGACCAAGCTGACCGTCCTA |
| DENV- | 11 | heavy | CAGGTGCAGCTGGTGCAGTCTGGGGCTGAGGTGAAGAAGCCTGGGGCCTCAGTGAAGGTCTCCTGCAAGGCTTCTGG |
| 298 | | | ATACACCTTCACCGGCTACCAGATGCACTGGGTGCGACAGGCCCCTGGCCAAGGGCTTGAGTGGATGGGATGGATCA |
| | | | ACCCTTACACCGGGGACACAAGTTATTCACAGAAGTTTCAGGGCAGGGTCACCATGACCCGGGACACGTCCATCAAC |
| | | | ACAGCCTACATGGAGCTGAACAGGCTGCGCCCTGACGACTCGGCCGTGTATTACTGTGCGAGATACGATTTCTGGAG |
| | | | TGTTCATATCTTTGACTTGTGGGGCCAGGGAACCCTGGTCACTGTCTCCTCA |
| | 12 | light | GACTTTGTGTTGACGCAGTCTCCAGGCACCCTGTCTTTATCTCCAGGGGAAAGAGCCACCCTCTCCTGCAGGGCCAG |
| | | | TCAGAGTGTTAGCAGCAGCTTCTTAGGCTGGTACCAGCAGAAACCTGGCCAGCCTCCCAGACTCCTCATCTATGGTG |
| | | | CATCCAGCAGGGCCACTGGCATCCCAGACAGGTTCAGTGGTAGTGGGTCTGGGACAGACTTCACTCTCACCATCAGC |
| | | | AGACTGGAGCCTGAAGATTTTGCAGTGTATTACTGTCAGCATTATGGTACCTCACCTGGGTTCACTTTCGGCCCTGG |
| | | | GACCAAAGTGGATATCAAA |
| DENV- | 13 | heavy | CAGGTGCAGCTGGTGCAGTCTGGGGCTGAGGTGAAGAAGCCTGGGGCCTCAGTGAAGGTCTCCTGCAAGGCTTCTGG |
| 354 | | | ATACACCTTCACCGGCTACCAGATGCACTGGGTGCGACAGGCCCCTGGCCAAGGGCTTGAGTGGATGGGATGGATCA |
| | | | ACCCTTACACCGGGGACACAAGTTATTCACAGAAGTTTCAGGGCAGGGTCACCATGACCCGGGACACGTCCATCAAC |
| | | | ACAGCCTACATGGAGCTGAACAGGCTGCGCCCTGACGACTCGGCCGTGTATTACTGTGCGAGATACGATTTCTGGAG |
| | | | TGTTCATATCTTTGACTTGTGGGGCCAGGGAACCCTGGTCACTGTCTCCTCA |
| | 14 | light | GACTTTGTGTTGACGCAGTCTCCAGGCACCCTGTCTTTATCTCCAGGGGAAAGAGCCACCCTCTCCTGCAGGGCCAG |
| | | | TCAGAGTGTTAGCAGCAGCTTCTTAGGCTGGTACCAGCAGAAACCTGGCCAGCCTCCCAGACTCCTCATCTATGGTG |
| | | | CATCCAGCAGGGCCACTGGCATCCCAGACAGGTTCAGTGGTAGTGGGTCTGGGACAGACTTCACTCTCACCATCAGC |
| | | | AGACTGGAGCCTGAAGATTTTGCAGTGTATTACTGTCAGCATTATGGTACCTCACCTGGGTTCACTTTCGGCCCTGG |
| | | | GACCAAAGTGGATATCAAA |
| DENV- | 15 | heavy | CAGGTGCAGCTGCAGGAGTCGGGCCCCGGACTGGTGAAGCCTTCACAGACCCTGTCCCTCACCTGCACTGTCTCTGG |
| 404 | | | TGGCTCCATGATCAGTGGTCGTTTCTACTGGAGCTGGATCCGGCAGCCCGCCGGGAAGGGACTGGAGTGGATTGGGC |
| | | | GTATATATAATAGTGGGAGCACCAATTACAACCCCTCCCTCAAAAGTCGAGTCACCTTATCACTGGACACGTCCAAG |
| | | | AACGAGTTCTCCCTGAAGCTGACCTCTGTGACCGCCGCAGACACGGCCGTGTACTACTGTGCGAAGGAGGACGATTT |
| | | | TTGGAGTGGCCATGGGGGGTTCGACCCCTGGGGCCAGGGAACCCCGGTCACCGTCTCCTCA |
| | 16 | light | GAAATAGTGATGACGCAGTCTCCAGCCACCCTGTCTGTGTCTCCAGGGGAAAGAGTCACCCTCTCCTGCAGGGCCAG |
| | | | TCAGAGTGTTCACATCAACGTAGCCTGGTACCACCAGAAACCTGGCCAGGCTCCCAGGCTCCTCATCTATGGTGCAT |
| | | | CCAAAAGGGCCACTGGTATCCCAGGCAGGTTCAGTGGCAGTGGGTCTGGGACAGAGTTCACTCTCACCATCAGCAGC |
| | | | CTGGAGTCTGAAGATTTTGCAGTGTATTTCTGTCAGCAGTATAACAACTGGCCCACTTTCGGCCCTGGGACCCAGGT |
| | | | GGATATCAAA |
| DENV- | 17 | heavy | GAAGTGCAGCTGGTGCAGTCTGGAGCAGAGGTGAAAAAGCCCGGGGAGTCTCTGAGGATCTCCTGTAAGGGTTCTGG |
| 406 | | | ATACAACTTTGCCAGCTACTGGATCACCTGGGTGCGCCAGATGCCCGGGAAAGGCCTGGAGTGGATGGGGAGGATTG |
| | | | ATCCTAGTGACTCTTATACCAACTACAGCCCGTCCTTCCAAGGCCACGTCACCATCTCAGCTGACAAGTCCATCAGC |
| | | | ACTGCCTACCTGCAGTGGAGCACCCTGAAGGCCTCGGACACCGCCATGTATTACTGTGCGCGATCGGGATCGTTTTG |
| | | | GAGTGCTTTTAACTGGTTCGACCCCTGGGGCCAGGGAACCCTGGTCACCGTCTCCTCA |
| | 18 | light | CAGTCTGCCCTGACGCAGCCTGCCTCCGTGTCTGGGTCTCCAGGACAGTCGATCACCATCTCCTGCACTGCAACCAG |
| | | | CAGTGAAATTGGGAGTTATAACCTTGTCTCCTGGTACCAACAACACCCAGGCAAAGCCCCCAAAGTCAAGATTTATG |
| | | | AGGGCACTAAGCGGCCCTCAGGGGTTTCAAATCGCTTCTCTGGCTCCAAGTCTGGCAATACGGCCTCCCTGACAATC |
| | | | TCTGGGCTCCAGGCTGAGGACGAGGCTGATTATTACTGCTGCTCATATGCAGGTAGTAACACTTGGGTGTTCGGCGG |
| | | | AGGGACCAAGCTGACCGTCCTA |
| DENV- | 19 | heavy | GAGGTGCAGGTGGTGGAGTCTGGGGGAGGCTTGGTCCAGCCGGGGGGGTCCCTGAGACTCTCCTGTGCAGCCTCTGG |
| 415 | | | ATTCACCTTTAGTTACTATTGGATGAGCTGGGTCCGCCAGGCTCCAGGGAAGGGGCTGGAGTGGGTGGCCAACATAA |
| | | | AGCAAGATGGAAGTGAGAAAAACTATGTGGACTCTGTGAAGGGCCGATTCACCATCTCCAGAGACAACGCCAAGAAC |
| | | | TCACTGTATCTGCAAATGAGCAGCCTGAGAGCCGAGGACACGGCCGTGTATTACTGTGCGAGAAGGGATTACGCTTT |
| | | | TTGGAGTGGTTATCGCTCTTTGTGGGACTACTGGGGCCAGGGAACCCTGGTCACCGTCTCCTCA |
| | 20 | light | GACATCCAGATGACCCAGTCTCCATCCTCCCTGTCTGCATCTGTGGGAGACAGAGTCACCATCACTTGCCGGGCAAA |
| | | | TCAAAGCATTAGTAGGTTTTTGAATTGGTATCAACACAAACCAGGGAAAGCCCCTAAGGTCCTGATCTACGCTGCTT |
| | | | CCAGTTTGCAAAGTGGTGTCCCCTCAAGGTTCAGTGGCAGTCAATCTGGGACAGATTTCACTCTCACCATCAGCAGT |
| | | | CTGCAACCTGAAGATTTTGGAACTTACTACTGTCAACAGAGTCACAGTCCCCCGGAGACGTTCGGCCAAGGGACCAA |
| | | | GGTGGAAATCAAA |
| DENV- | 21 | heavy | GAAATGCAGCTGCAGGAGTCGGGCCCAAGACTGGTGAAGCCTTCACAGACCCTGTCCCTCACCTGCACTGTCTCTGG |
| 419 | | | TGGCTCCACCAGCAGTGGTGGTTACTACTGGAGCTGGTTCCGCCAGTACCCCGAGAAGGGCCTGGAGTGGATTGGGT |
| | | | ACATCTTTAGTAGTGTGACCACCTACTACAACCCGCCCCTCAAGAGTCGAGTCACCATATCAGTAGACACGTCTAAG |
| | | | AACCAGTTCTCCCTGAAGTTGAGCTCTGTCACTGCCGCGGACACGGCCGTGTATTACTGTGCGAGATCTTTACATTA |
| | | | CTATCAGAGTAGTGGTTTCCTCTACTGGGGCCGGGGAATCCCGGTCACCGTCTCCGCA |
| | 22 | light | GAAATTGTGTTGACACAGTCTCCAGCCACCCTGTCTTTGTCTCCAGGGGATAGAGCCACCCTCTCCTGCAGGGCCAG |
| | | | TCAGACTGTTGGCGACTCCTTGGCCTGGTACCAACAAAAACCTGGCCAGGCTCCCAGGCTCCTCATCTATGATGTTT |
| | | | CCAACAGGGCCACTGGCATCCCAGCCAGGTTCAGTGGCAGTGGGTCTGGGACAGACTTCACTCTCACCATCAGCAGC |
| | | | CTAGAGCCTGAGGATTTTGCAGTTTATTACTGTCAGCAGCGTGCCAGCTTCGTCACCTTCGGCAGAGGGACCAAGGT |
| | | | GGACATCAAA |
| DENV- | 23 | heavy | CAGGTGCAGCTGCAGGAGTCGGGCCCAGGACTGGTGAAGCCTTCGGAGACCCTGTCCCTCACCTGCAGTGTCTCTGG |
| 437 | | | TGCCTCCATCAGGAGTGACTACTGGATCTGGATCCGGCAGCCCGCCGGGAAGGGACTGGAGTGGATTGGGCGTATTG |
| | | | ACACCACTGGGAAGACCAACTACAACCCCTCCCTTAAGAGTCGAGTCACCATGTCAGTTGACACGTCCAAGAACCAG |
| | | | TTCTCCCTGAAGCTGAGGTCTGTGACCGCCGCGGACACGGCCGTGTATTATTGTGCGAGAGAATACCGACTACGGTG |
| | | | GACAGTGTACTACTTTGACTACTGGGGCCAGGGAACCCTGGTCACCGTCTCCTCA |
| | 24 | light | GAAATAGTGATGACGCAGTCTCCAGCCACCCTGTCTGTGTCTCCAGGGGAAAGAGCCACCCTCTCTTGCAGGGCCAG |
| | | | TCAGAGTGTTAGCAGCAACTTAGCCTGGTACCAGCAGAGACCTGGCCAGGCTCCCAGGCTCCTCATCTATGGTGCAT |
| | | | CCACCAGGGCCACTGGTATCCCAGCCAGGTTCACTGGCAGTGGGTCTGGGACAGAGTTCACCCTCACCATCAGCAGC |
| | | | CTGCAGTCTGAAGATTTTGCAGTTTATTACTGTCAGCAGTATAGTGACTGGTTTCAGCTCACTTTCGGCGGAGGGAC |
| | | | CAAGGTGGAGATCAAA |
| DENV- | 25 | heavy | CAGGTGCAGCTGGTGGAGTCTGGGGGAGGCTTGGTCAAGCCTGGAGGGTCCCTGAGACTCTCCTGTGCAGCCTCTGG |
| 443 | | | ATTCAGCTTCAGTGACTTCCACATGAGCTGGATCCGTCAGGCTCCAGGGAAGGGGCTGGAGTGGGTATCATATATTA |
| | | | GTAGTAGTAGTCTTTCCACAAAGTACGCAGACTCTGTGAAGGGCCGATTCACCATCTCCAGAGACAACGCCAAGAAC |
| | | | TCACTGTATCTGCAAATGAACAGCCTGAGAGCCGAGGACACGGCCGTGTATTACTGTGCGAGATGGGACATTATTAC |
| | | | CATGATAGTCGTACTTGGGGATGCTGTTGATATCTGGGGCCAAGGGACAAAGGTCACCGTCTCTTCA |
| | 26 | light | CAGACTGTGGTGACTCAGGAGCCCTCACTGACTGTGTCCCCAGGAGGGACAGTCACTCTCACCTGCGCTTCCAGCAC |
| | | | TGGAGCAGTCACCAGGACTTTCTATCCAAACTGGTTCCAGCAGAAACCTGGACAAGCACCCAGGGCACTGATTTATA |
| | | | GTACAAGCAAAAAACACTCCTGGACCCCTGCCCGGTTCTCAGGCTCCCTCCTTGGGGGCAAAGCTGCCCTGACACTG |
| | | | TCGGGTGTGCAGCCTGAGGACGAGGCTGAGTATCACTGCCTGCTCTACTATTATGGTGCCCAGCTTTGGGTGTTCGG |
| | | | CGGAGGGACCAAGCTGACCGTCCTA |

TABLE 2
PROTEIN SEQUENCES FOR ANTIBODY VARIABLE REGIONS

| Clone | SEQ ID NO: | Chain | Variable Sequence |
|---|---|---|---|
| DENV-115 | 27 | heavy | QVQLVQSGAEVKKPGAPVKVSCEASGYTFTDYFIHWVRQAPGQGLEWMGWINPI |
| | | | SGGTNYHPRFHGGVTMTRDTSMKVAYMELKRLTSDDTAVYFCARGRDFRGGYSQ |
| | | | LDYWGQGTLVTVSS |
| | 28 | light | QSVLTQPPSASGTPGQRVTISCSGGSSNIAINTVNWYQQVPGTAPKLLMYSNNQ |
| | | | RPSGVPDRFSGSKSGTSASLAISGLQSEDEADYYCATWDDSLKDVLFGGGTKLT |
| | | | VL |
| DENV-144 | 29 | heavy | QVQLQESGPGLVKPSETLSLTCTVSGASISSYSWSWIRQPAGRGLEWLGRIYPS |
| | | | YPSGNTNYSPSLKSRLTMSLDTSKNQFSMKLTSVSAADTAVYYCARDREQWPLY |
| | | | YGMDVWGQGTLVTVSS |
| | 30 | light | EIVLTQSPAILSLSPGDRATLSCRASQSVFTYLAWYQHKPGQAPRLLIYDASNR |
| | | | ASGIPARFSGSGSGTDFTLTISSLEPEDFAVYYCQQRTKWPLAFGGGTKVEIK |
| DENV-286 | 31 | heavy | QVQLQESGPGLVKPSETLSLSCTVSGGSISPDYWSWIRQPPGKGLEWLGYIYSA |
| | | | GSTSYNPSLKSRVTMSVDTSKNQLSLKLTSVTAADTAVYYCARTAGSFWSGRGW |
| | | | FDPWGQGTLVTVSS |
| | 32 | light | EIVLTQSPATLSLSPGERVTLSCGASQSVSSSHLAWYQQKPGLAPRLLIYDASN |
| | | | RATGVPDRFSGSGSGTDFTLTISRLEPEDFAVYYCQQYGSPQYTFGQGTKLEIK |
| | | | RTVAAP |
| DENV-290 | 33 | heavy | QCQVELVESGGDVVQPGKSLRLSCAASGFTFTNYAMHWLRQAPGKGLEWVAVIS |
| | | | SDVNDKYYADSVKGRFTISRDNSKNTLYLQMNSLTPEDTAVYYCAREQAVGTNP |
| | | | WAFDYWGQGTLVTVSS |
| | 34 | light | HIVMTQSPLSLSVTPGQPASISCKSSQISSWGSDGKTYLYWYLQKPGQSPQLLI |
| | | | YEVSSRFSGVSDRFSGSGSGTDFTLKISRVQAEDVGLYYCMQGLHLPLTFGQGT |
| | | | RLEIK |
| DENV-297 | 35 | heavy | EVQLLESGGGLVQPGGSLRLSCAASGFTFNNSAMGSYAMIWVRQAPGKGLEWVS |
| | | | TITGTGLTTYYADSVKGRFTVSRDNSRNTLHLQMNSLRAEDTAVYYCAKWNIIT |
| | | | MAPFDIWGQGTLVTVSS |
| | 36 | light | QTVVTQEPSFSVSPGGTVTLTCGLTSGSVSTSYYTSWYQQTPGQAPRTLIYKTN |
| | | | TRSSGVPDRFSGSIVGNKAALTITGAQPDDESDYYCVLYVGSGIWVFGGGTKLT |
| | | | VL |
| DENV-298 | 37 | heavy | QVQLVQSGAEVKKPGASVKVSCKASGYTFTGYQMHWVRQAPGQGLEWMGWINPY |
| | | | TGDTSYSQKFQGRVTMTRDTSINTAYMELNRLRPDDSAVYYCARYDFWSVHIFD |
| | | | LWGQGTLVTVSS |
| | 38 | light | DFVLTQSPGTLSLSPGERATLSCRASQSVSSSFLGWYQQKPGQPPRLLIYGASS |
| | | | RATGIPDRFSGSGSGTDFTLTISRLEPEDFAVYYCQHYGTSPGFTFGPGTKVDI |
| | | | K |
| DENV-354 | 39 | heavy | QVQLVQSGAEVKKPGASVKVSCKASGYTFTGYQMHWVRQAPGQGLEWMGWINPY |
| | | | TGDTSYSQKFQGRVTMTRDTSINTAYMELNRLRPDDSAVYYCARYDFWSVHIFD |
| | | | LWGQGTLVTVSS |
| | 40 | light | DFVLTQSPGTLSLSPGERATLSCRASQSVSSSFLGWYQQKPGQPPRLLIYGASS |
| | | | RATGIPDRFSGSGSGTDFTLTISRLEPEDFAVYYCQHYGTSPGFTFGPGTKVDI |
| | | | K |
| DENV-404 | 41 | heavy | QVQLQESGPGLVKPSQTLSLTCTVSGGSMISGRFYWSWIRQPAGKGLEWIGRIY |
| | | | NSGSTNYNPSLKSRVTLSLDTSKNEFSLKLTSVTAADTAVYYCAKEDDFWSGHG |
| | | | GFDPWGQGTPVTVSS |
| | 42 | light | EIVMTQSPATLSVSPGERVTLSCRASQSVHINVAWYHQKPGQAPRLLIYGASKR |
| | | | ATGIPGRFSGSGSGTEFTLTISSLESEDFAVYFCQQYNNWPTFGPGTQVDIK |
| DENV-406 | 43 | heavy | EVQLVQSGAEVKKPGESLRISCKGSGYNFASYWITWVRQMPGKGLEWMGRIDPS |
| | | | DSYTNYSPSFQGHVTISADKSISTAYLQWSTLKASDTAMYYCARSGSFWSAFNW |
| | | | FDPWGQGTLVTVSS |
| | 44 | light | QSALTQPASVSGSPGQSITISCTATSSEIGSYNLVSWYQQHPGKAPKVKIYEGT |
| | | | KRPSGVSNRFSGSKSGNTASLTISGLQAEDEADYYCCSYAGSNTWVFGGGTKLT |
| | | | VL |
| DENV-415 | 45 | heavy | EVQVVESGGGLVQPGGSLRLSCAASGFTFSYYWMSWVRQAPGKGLEWVANIKQD |
| | | | GSEKNYVDSVKGRFTISRDNAKNSLYLQMSSLRAEDTAVYYCARRDYAFWSGYR |
| | | | SLWDYWGQGTLVTVSS |
| | 46 | light | DIQMTQSPSSLSASVGDRVTITCRANQSISRFLNWYQHKPGKAPKVLIYAASSL |
| | | | QSGVPSRFSGSQSGTDFTLTISSLQPEDFGTYYCQQSHSPPETFGQGTKVEIK |
| DENV-419 | 47 | heavy | EMQLQESGPRLVKPSQTLSLTCTVSGGSTSSGGYYWSWFRQYPEKGLEWIGYIF |
| | | | SSVTTYYNPPLKSRVTISVDTSKNQFSLKLSSVTAADTAVYYCARSLHYYQSSG |
| | | | FLYWGRGIPVTVSA |
| | 48 | light | EIVLTQSPATLSLSPGDRATLSCRASQTVGDSLAWYQQKPGQAPRLLIYDVSNR |
| | | | ATGIPARFSGSGSGTDFTLTISSLEPEDFAVYYCQQRASFVTFGRGTKVDIK |
| DENV-437 | 49 | heavy | QVQLQESGPGLVKPSETLSLTCSVSGASIRSDYWIWIRQPAGKGLEWIGRIDTT |
| | | | GKTNYNPSLKSRVTMSVDTSKNQFSLKLRSVTAADTAVYYCAREYRLRWTVYYF |
| | | | DYWGQGTLVTVSS |
| | 50 | light | EIVMTQSPATLSVSPGERATLSCRASQSVSSNLAWYQQRPGQAPRLLIYGASTR |
| | | | ATGIPARFTGSGSGTEFTLTISSLQSEDFAVYYCQQYSDWFQLTFGGGTKVEIK |
| DENV-443 | 51 | heavy | QVQLVESGGGLVKPGGSLRLSCAASGFSFSDFHMSWIRQAPGKGLEWVSYISSS |
| | | | SLSTKYADSVKGRFTISRDNAKNSLYLQMNSLRAEDTAVYYCARWDIITMIVVL |
| | | | GDAVDIWGQGTKVTVSS |
| | 52 | light | QTVVTQEPSLTVSPGGTVTLTCASSTGAVTRTFYPNWFQQKPGQAPRALIYSTS |
| | | | KKHSWTPARFSGSLLGGKAALTLSGVQPEDEAEYHCLLYYYGAQLWVFGGGTKL |
| | | | TVL |

TABLE 3
HEAVY CHAIN CDR SEQUENCES

| | CDRH1 | CDRH2 | CDRH3 |
|---|---|---|---|
| Clone | (SEQ ID NO:) | (SEQ ID NO:) | (SEQ ID NO:) |
| DENV- | GYTFTDYF | INPISGGT | ARGRDFRGGYSQLDY |
| 115 | 53 | 54 | 55 |
| DENV- | GASISSYS | IYPSGNT | ARDREQWPLYYGMDV |
| 144 | 56 | 57 | 58 |
| DENV- | GGSISPDY | IYSAGST | ARTAGSFWSGRGWFDP |
| 286 | 59 | 60 | 61 |
| DENV- | GFTFTNYA | ISSDVNDK | AREQAVGTNPWAFDY |
| 290 | 62 | 63 | 64 |
| DENV- | GFTFNNSAMGSYA | ITGTGLTT | AKWNIITMAPFDI |
| 297 | 65 | 66 | 67 |
| DENV- | GYTFTGYQ. | INPYTGDT | ARYDFWSVHIFDL |
| 298 | 68 | 69 | 70 |
| DENV- | GYTFTGYQ. | INPYTGDT | ARYDFWSVHIFDL |
| 354 | 71 | 72 | 73 |
| DENV- | GGSMISGRFY | IYNSGST | AKEDDFWSGHGGFDP |
| 404 | 74 | 75 | 76 |
| DENV- | GYNFASYW | IDPSDSYT | ARSGSFWSAFNWFDP |
| 406 | 77 | 78 | 79 |
| DENV- | GFTFSYYW | IKQDGSEK | ARRDYAFWSGYRSLWDY |
| 415 | 80 | 81 | 82 |
| DENV- | GGSTSSGGYY | IFSSVTT | ARSLHYYQSSGFLY |
| 419 | 83 | 84 | 85 |
| DENV- | GASIRSDY | IDTTGKT | AREYRLRWTVYYFDY |
| 437 | 86 | 87 | 88 |
| DENV- | GFSFSDFH | ISSSSLST | ARWDIITMIVVLGDAVDI |
| 443 | 89 | 90 | 91 |

TABLE 4
LIGHT CHAIN CDR SEQUENCES

| | CDRL1 | CDRL2 | CDRL3 |
|---|---|---|---|
| Clone | (SEQ ID NO:) | (SEQ ID NO:) | (SEQ ID NO:) |
| DENV-115 | SSNIAINT | SNN | ATWDDSLKDVL |
| | 92 | 93 | 94 |
| DENV-144 | QSVFTY | DAS | QQRTKWPLA |
| | 95 | 96 | 97 |
| DENV-286 | QSVSSSH | DAS | QQYGSPQYT |
| | 98 | 99 | 100 |
| DENV-290 | QISSWGSDGKTY | EVS | MQGLHLPLT |
| | 101 | 102 | 103 |
| DENV-297 | SGSVSTSYY | KTN | VLYVGSGIWV |
| | 104 | 105 | 106 |
| DENV-298 | QSVSSSF | GAS | QHYGTSPGFT |
| | 107 | 108 | 109 |
| DENV-354 | QSVSSSF | GAS | QHYGTSPGFT |
| | 110 | 111 | 112 |
| DENV-404 | QSVHIN | GAS | QQYNNWPT |
| | 113 | 114 | 115 |
| DENV-406 | SSEIGSYNL | EGT | CSYAGSNTWV |
| | 116 | 117 | 118 |
| DENV-415 | QSISRF | AAS | QQSHSPPET |
| | 119 | 120 | 121 |
| DENV-419 | QTVGDS | DVS | QQRASFVT |
| | 122 | 123 | 124 |
| DENV-437 | QSVSSN | GAS | QQYSDWFQLT |
| | 125 | 126 | 127 |
| DENV-443 | TGAVTRTFY | STS | LLYYYGAQLWV |
| | 128 | 129 | 130 |

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VII. REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,196,265
U.S. Pat. No. 4,275,149
U.S. Pat. No. 4,277,437
U.S. Pat. No. 4,366,241
U.S. Pat. No. 4,472,509
U.S. Pat. No. 4,554,101
U.S. Pat. No. 4,680,338
U.S. Pat. No. 4,816,567
U.S. Pat. No. 4,867,973
U.S. Pat. No. 4,938,948
U.S. Pat. No. 5,021,236
U.S. Pat. No. 5,141,648
U.S. Pat. No. 5,196,066
U.S. Pat. No. 5,563,250
U.S. Pat. No. 5,565,332
U.S. Pat. No. 5,856,456
U.S. Pat. No. 5,880,270
U.S. Pat. No. 6,485,982
“Antibodies: A Laboratory Manual,” Cold Spring Harbor Press, Cold Spring Harbor, N.Y., 1988.
Abbondanzo et al., Am. J. Pediatr. Hematol. Oncol., 12(4), 480-489, 1990.
Allred et al., Arch. Surg., 125(1), 107-113, 1990.
Andrade and Widman, D. Sci Rep. 7(1):17169, 2017.
Atherton et al., Biol. of Reproduction, 32, 155-171, 1985.
Barzon et al., Euro Surveill. 2016 Aug. 11; 21(32).
Beltramello et al., Cell Host Microbe 8, 271-283, 2010.
Bhatt et al., Nature 496, 504-507, 2013.
Brown et al., J. Immunol. Meth., 12;130(1), :111-121, 1990.
Campbell, In: Monoclonal Antibody Technology, Laboratory Techniques in Biochemistry and Molecular Biology, Vol. 13, Burden and Von Knippenberg, Eds. pp. 75-83, Amsterdam, Elsevier, 1984.
Capaldi et al., Biochem. Biophys. Res. Comm., 74(2):425-433, 1977.
Capeding et al., Lancet 384, 1358-1365, 2014.
De Jager et al., Semin. Nucl. Med. 23(2), 165-179, 1993.
Dejnirattisai et al., Nat Immunol. 16(2):170-177, 2015.
Dholakia et al., J. Biol. Chem., 264, 20638-20642, 1989.
Diamond et al., J Virol 77, 2578-2586, 2003.
Doolittle and Ben-Zeev, Methods Mol. Biol., 109, :215-237, 1999.
Duffy et al., N. Engl. J. Med. 360, 2536-2543, 2009.
Elder et al. Infections, infertility and assisted reproduction. Part II: Infections in reproductive medicine & Part III: Infections and the assisted reproductive laboratory. Cambridge UK: Cambridge University Press; 2005.
Gallichotte et al., MBio. 6(5):e01461-15, 2015.
Gefter et al., Somatic Cell Genet., 3:231-236, 1977.
Gornet et al., Semin Reprod Med. 2016 Sep; 34(5):285-292. Epub 2016 Sep 14.
Gulbis and Galand, Hum. Pathol. 24(12), 1271-1285, 1993.
Halfon et al., PLoS ONE 2010; 5 (5) e10569
Hessell et al., Nature 449, 101-4, 2007.
Khatoon et al., Ann. of Neurology, 26, 210-219, 1989.
King et al., J. Biol. Chem., 269, 10210-10218, 1989.
Kohler and Milstein, Eur. J. Immunol., 6, 511-519, 1976.
Kohler and Milstein, Nature, 256, 495-497, 1975.
Kyte and Doolittle, J. Mol. Biol., 157(1):105-132, 1982.
Mansuy et al., Lancet Infect Dis. 16(10):1106-7, 2016.
Messer et al., PLoS Negl Top Dis 6, 2012.
Messer et al., J Virol. 90(10):5090-5097, 2016.
Nakamura et al., In: Enzyme Immunoassays: Heterogeneous and Homogeneous Systems, Chapter 27, 1987.
O'Shannessy et al., J. Immun. Meth., 99, 153-161, 1987.
Persic et al., Gene 187:1, 1997
Potter and Haley, Meth. Enzymol., 91, 613-633, 1983.
Purpura et al., Lancet Infect Dis. 2016 Oct; 16(10):1107-8. Epub 2016 Sep 19.
Remington's Pharmaceutical Sciences, 15th Ed., 3:624-652, 1990.
Smith et al., J Virol. 86(5):2665-75, 2012.
Smith et al., J Infect Dis. 207(12):1898-908, 2013.
Tang et al., J. Biol. Chem., 271, 28324-28330, 1996.
Thomas and Endy, Curr Opin Infect Dis 24, 442-450, 2011.
Villar et al., N Engl J Med, NEJM 372(2), 113-123, 2014.
Wawrzynczak & Thorpe, In: Immunoconjugates, Antibody Conjugates In Radioimaging And Therapy Of Cancer, Vogel (Ed.), NY, Oxford University Press, 28, 1987.
Yu et al., J Immunol Methods 336, 142-151, doi:10.1016/j.jim.2008.04.008, 2008.
Andrade et al., MBio 8. e01205-17, 2017.
Andrade et al., Sci Rep 9, 16258, 2019.
Balmaseda et al., Clin Diagn Lab Immunol 10, 317-322, 2003.
Balmaseda et al., Trop Med Int Health 11, 935-942, 2006.
Balmaseda et al., Am J Trop Med Hyg 61, 893-897, 1999.
Balmaseda et al., J Infect Dis 201, 5-14, 2010.
Balsitis et al., PLoS Pathog 6, e1000790, 2010.
Beltramello et al., Cell Host Microbe 8, 271-283, 2010.
Bhatt et al., Nature 496, 504-507, 2013.
Bhaumik et al., Viruses 11. E19, 2018.
Biswal et al., N Engl J Med 381, 2009-2019, 2019.
Brien et al., J Virol 84, 10630-10643, 2010.
Chen, R., and Vasilakis, N., Viruses 3, 1562-1608, 2011.
Collins et al., JCI Insight 4. 124588, 2019.
de Alwis et al., Proc Natl Acad Sci U S A 109, 7439-7444, 2012.
de Alwis et al., PLoS Pathog 10, e1004386, 2014.
de Silva et al., Cold Spring Harb Perspect Biol 10. a029371, 2018
Dejnirattisai et al., Nat Immunol 16, 170-177, 2015a.
Dejnirattisai et al., Nat Immunol 16, 785, 2015b.
Diamond, M. S., and Pierson, T. C., Cell 162, 488-492, 2015.
Ferguson et al., Science 353, 1033-1036, 2016.
Fernandez, R. J., and Vazquez, S., Mem Inst Oswaldo Cruz 85, 347-351, 1990.
Fibriansah et al., Science 349, 88-91, 2015a.
Fibriansah et al., EMBO Mol Med 6, 358-371, 2014.
Fibriansah et al., Nat Commun 6, 6341, 2015b.
Fleith et al., Sci Rep 6, 36339, 2016.
Flipse, J., and Smit, J. M., PLoS Negl Trop Dis 9, e0003749, 2015.
Gallichotte et al., Adv Exp Med Biol 1062, 63-76, 2018a.
Gallichotte et al., Cell Rep 25, 1214-1224, 2018b.
Gallichotte et al., PLoS Pathog 14, e1006934, 2018c.
Gallichotte et al., mSphere 2. e00380-16. 2017
Gallichotte et al., MBio 6, e01461-01415, 2015.
Gallichotte et al., MBio 10. e01461-15, 2019.
Grifoni et al., J Virol 91. e01469-17, 2017
Gutierrez et al., PLoS Negl Trop Dis 5, e1394, 2011.
Halstead, S. B. F1000Res 4, 2015.
Halstead, S. B. Vaccine 35, 6355-6358, 2017.
Halstead, S. B. Hum Vaccin Immunother 14, 2158-2162, 2018a.
Halstead, S. B. Cold Spring Harb Perspect Biol 10. a030700, 2018b.
Halstead et al., Nat New Biol 243, 24-26, 1973.
Hasan et al., Nat Commun 8, 14722, 2017.
Henein et al., J Infect Dis 215, 351-358, 2017.
Holmes, E. C., and Twiddy, S. S. Infect Genet Evol 3, 19-28, 2003.
Johnson, A. J., and Roehrig, J. T. J Virol 73, 783-786, 1999.
Katzelnick et al., Science 349, 1338-1343, 2015.
Katzelnick et al., Science 358, 929-932, 2017a.
Katzelnick et al., Vaccine 35, 4659-4669, 2017b.
Katzelnick et al., Proc Natl Acad Sci U S A 113, 728-733, 2016.
King et al., Virol J 5, 63, 2008.
Kraus et al., J Clin Microbiol 45, 3777-3780, 2007.
Kuan et al., Am J Epidemiol 170, 120-129, 2009.
Kudlacek et al., J Biol Chem 293, 8922-8933, 2018.
Kuhn et al., Virology 479-480, 508-517, 2015.
Lanciotti et al., J Clin Microbiol 30, 545-551, 1992.
Lee et al., J Virol 87, 13729-13740, 2013.
Li et al., Structure 26, 51-59 e54, 2018.
Li et al., PLoS Pathog 15, e1007716, 2019.
Lofano et al., Sci Immunol 3. eaat7796, 2018.
Long et al., Proc Natl Acad Sci U S A 116, 1591-1596, 2019.
Magnani et al., J Virol 91. e00867-17, 2017.
Mattia et al., PLoS One 6, e27252, 2011.
Messer et al., PLoS Negl Trop Dis 6, e1486, 2012.
Messer et al., J Virol 90, 5090-5097, 2016.
Metz et al., Sci Rep 7, 4524, 2017.
Michlmayr et al., Nat Microbiol 2, 1462-1470, 2017.
Montoya et al., PLoS Negl Trop Dis 7, e2357, 2013.
Moodie et al., J Infect Dis 217, 742-753, 2018.
Mukherjee et al., J Virol 88, 7210-7220, 2014.
Murphy, B.R., and Whitehead, S.S. Annu Rev Immunol 29, 587-619, 2011.
Nivarthi et al., J Virol 91. e02041-16, 2017
OhAinle et al., Sci Transl Med 3, 114ra128, 2011.
Patel et al., PLoS Negl Trop Dis 11, e0005554, 2017.
Pierson, T. C., and Diamond, M. S. Expert Rev Mol Med 10, e12, 2008.
Pierson, T. C., and Diamond, M. S. Prog Mol Biol Transl Sci 129, 141-166, 2015.
Raekiansyah, et al., Southeast Asian J Trop Med Public Health 36, 1187-1197, 2005.
Robbiani et al., Cell 169, 597-609 e511, 2017.
Rouvinski et al., Nat Commun 8, 15411, 2017.
Salje et al., Nature 557, 719-723, 2018.
Sangkawibha et al., Am J Epidemiol 120, 653-669, 1984.
Sapparapu et al., Nature 540, 443-447, 2016.
Sarathy et al., Sci Rep 8, 4900, 2018.
Shresta et al., J Virol 78, 2701-2710, 2004.
Shresta et al., J Virol 80, 10208-10217, 2006.
Simon et al., Proc Biol Sci 282, 20143085, 2015.
Smith et al., J Virol 88, 12233-12241, 2014.
Smith et al., J Infect Dis 207, 1898-1908, 2013.
Smith et al., J Virol 86, 2665-2675, 2012.
Sridhar et al., N Engl J Med 379, 327-340, 2018.
Stettler et al., Science 353, 823-826, 2016.
Sukupolvi-Petty et al., J Virol 87, 8826-8842, 2013.
Swanstrom et al., J Infect Dis 217, 1932-1941, 2018.
Swanstrom et al., J Infect Dis 220, 219-227, 2019.
Swanstrom et al., MBio 7. e01123-16, 2016.
Teoh et al., Sci Transl Med 4, 139ra183, 2012.
Waggoner et al., J Infect Dis 214, 986-993, 2016.
Wahala et al., PLoS Pathog 6, e1000821, 2010.
Waman et al., Infect Genet Evol 49, 234-240, 2017.
Wang, et al., Cell 171, 229-241 e215, 2017.
Widman et al., Sci Rep 7, 17169, 2017.
Yu et al., J Immunol Methods 336, 142-151, 2008.
Zhao et al., J Exp Med 217. e20191792, 2020.
Zompi et al., PLoS Negl Trop Dis 6, e1568, 2012.

1. A method of detecting a dengue virus infection in a subject comprising:
(a) contacting a sample from said subject with an antibody or antibody fragment having clone-paired heavy and light chain CDR sequences from Tables 3 and 4, respectively; and
(b) detecting dengue virus in said sample by binding of said antibody or antibody fragment to a Dengue virus antigen in said sample.

2. The method of claim 1, wherein said sample is a body fluid.

3. The method of claim 1, wherein said sample is blood, sputum, tears, saliva, mucous or serum, semen, cervical or vaginal secretions, amniotic fluid, placental tissues, urine, exudate, transudate, tissue scrapings or feces.

4. The method of claim 1, wherein detection comprises ELISA, RIA, lateral flow assay or Western blot.

5. The method of claim 1, further comprising performing steps (a) and (b) a second time and determining a change in dengue virus antigen levels as compared to the first assay.

6. The method of claim 1, wherein the antibody or antibody fragment is encoded by clone-paired variable sequences as set forth in Table 1.

7. The method of claim 1, wherein said antibody or antibody fragment is encoded by light and heavy chain variable sequences having 70%, 80%, or 90% identity to clone-paired variable sequences as set forth in Table 1.

8. The method of claim 1, wherein said antibody or antibody fragment is encoded by light and heavy chain variable sequences having 95% identity to clone-paired sequences as set forth in Table 1.

9. The method of claim 1, wherein said antibody or antibody fragment comprises light and heavy chain variable sequences according to clone-paired sequences from Table 2.

10. The method of claim 1, wherein said antibody or antibody fragment comprises light and heavy chain variable sequences having 70%, 80% or 90% identity to clone-paired sequences from Table 2.

11. The method of claim 1, wherein said antibody or antibody fragment comprises light and heavy chain variable sequences having 95% identity to clone-paired sequences from Table 2.

12. The method of claim 1, wherein the antibody fragment is a recombinant scFv (single chain fragment variable) antibody, Fab fragment, F(ab′)₂ fragment, or Fv fragment.

13. A method of treating a subject infected with dengue virus or reducing the likelihood of infection of a subject at risk of contracting dengue virus comprising delivering to said subject an antibody or antibody fragment having clone-paired heavy and light chain CDR sequences from Tables 3 and 4, respectively.

14. The method of claim 13, the antibody or antibody fragment is encoded by clone-paired light and heavy chain variable sequences as set forth in Table 1.

15. The method of claim 13, the antibody or antibody fragment is encoded by clone-paired light and heavy chain variable sequences having 95% identity to as set forth in Table 1.

16. The method of claim 13, wherein said antibody or antibody fragment is encoded by light and heavy chain variable sequences having 70%, 80%, or 90% identity to clone-paired sequences from Table 1.

17. The method of claim 13, wherein said antibody or antibody fragment comprises light and heavy chain variable sequences according to clone-paired sequences from Table 2.

18. The method of claim 13, wherein said antibody or antibody fragment comprises light and heavy chain variable sequences having 70%, 80% or 90% identity to clone-paired sequences from Table 2.

19. The method of claim 13, wherein said antibody or antibody fragment comprises light and heavy chain variable sequences having 95% identity to clone-paired sequences from Table 2.

20. The method of claim 13, wherein the antibody fragment is a recombinant scFv (single chain fragment variable) antibody, Fab fragment, F(ab′)₂ fragment, or Fv fragment.

21. The method of claim 13, wherein said antibody is an IgG, or a recombinant IgG antibody or antibody fragment comprising an Fc portion mutated to alter (eliminate or enhance) FcR or FcRn interactions, to increase half-life and/or increase therapeutic efficacy, such as a LALA, LALA PG, N297, GASD/ALIE, DHS, YTE or LS mutation or glycan modified to alter (eliminate or enhance) FcR or FcRn interactions such as enzymatic or chemical addition or removal of glycans or expression in a cell line engineered with a defined glycosylating pattern.

22. The method of claim 13, wherein said antibody is a chimeric antibody or a bispecific antibody.

23. The method of claim 13, wherein said antibody or antibody fragment is administered prior to infection or after infection.

24. The method of claim 13, wherein said subject is a pregnant female, a sexually active female, or a female undergoing fertility treatments.

25. The method of claim 13, wherein delivering comprises antibody or antibody fragment administration, or genetic delivery with an RNA or DNA sequence or vector encoding the antibody or antibody fragment.

26. A monoclonal antibody, wherein the antibody or antibody fragment is characterized by clone-paired heavy and light chain CDR sequences from Tables 3 and 4, respectively.

27. The monoclonal antibody of claim 26, wherein said antibody or antibody fragment is encoded by light and heavy chain variable sequences according to clone-paired sequences from Table 1.

28. The monoclonal antibody of claim 26, wherein said antibody or antibody fragment is encoded by light and heavy chain variable sequences having at least 70%, 80%, or 90% identity to clone-paired sequences from Table 1.

29. The monoclonal antibody of claim 26, wherein said antibody or antibody fragment is encoded by light and heavy chain variable sequences having at least 95% identity to clone-paired sequences from Table 1.

30. The monoclonal antibody of claim 26, wherein said antibody or antibody fragment comprises light and heavy chain variable sequences according to clone-paired sequences from Table 2.

31. The monoclonal antibody of claim 26, wherein said antibody or antibody fragment comprises light and heavy chain variable sequences having 95% identity to clone-paired sequences from Table 2.

32. The monoclonal antibody of claim 26, wherein the antibody fragment is a recombinant scFv (single chain fragment variable) antibody, Fab fragment, F(ab′)₂ fragment, or Fv fragment.

33. The monoclonal antibody of claim 26, wherein said antibody is a chimeric antibody, or is bispecific antibody.

34. The monoclonal antibody of claim 26, wherein said antibody is an IgG, or a recombinant IgG antibody or antibody fragment comprising an Fc portion mutated to alter (eliminate or enhance) FcR or FcRn interactions, to increase half-life and/or increase therapeutic efficacy, such as a LALA, LALA PG, N297, GASD/ALIE, DHS, YTE or LS mutation or glycan modified to alter (eliminate or enhance) FcR or FcRn interactions such as enzymatic or chemical addition or removal of glycans or expression in a cell line engineered with a defined glycosylating pattern.

35. The monoclonal antibody of claim 26, wherein said antibody or antibody fragment further comprises a cell penetrating peptide and/or is an intrabody.

36. A hybridoma or engineered cell encoding an antibody or antibody fragment wherein the antibody or antibody fragment is characterized by clone-paired heavy and light chain CDR sequences from Tables 3 and 4, respectively.

37. The hybridoma or engineered cell of claim 36, wherein said antibody or antibody fragment is encoded by light and heavy chain variable sequences according to clone-paired sequences from Table 1.

38. The hybridoma or engineered cell of claim 36, wherein said antibody or antibody fragment is encoded by light and heavy chain variable sequences having at least 70%, 80%, or 90% identity to clone-paired variable sequences from Table 1.

39. The hybridoma or engineered cell of claim 36, wherein said antibody or antibody fragment is encoded by light and heavy chain variable sequences having 95% identity to clone-paired variable sequences from Table 1.

40. The hybridoma or engineered cell of claim 36, wherein said antibody or antibody fragment comprises light and heavy chain variable sequences according to clone-paired sequences from Table 2.

41. The hybridoma or engineered cell of claim 36, wherein said antibody or antibody fragment is encoded by light and heavy chain variable sequences having at least 70%, 80%, or 90% identity to clone-paired variable sequences from Table 2.

42. The hybridoma or engineered cell of claim 36, wherein said antibody or antibody fragment comprises light and heavy chain variable sequences having 95% identity to clone-paired sequences from Table 2.

43. The hybridoma or engineered cell of claim 36, wherein the antibody fragment is a recombinant scFv (single chain fragment variable) antibody, Fab fragment, F(ab′)₂ fragment, or Fv fragment.

44. The hybridoma or engineered cell of claim 36, wherein said antibody is a chimeric antibody or a bispecific antibody.

45. The hybridoma or engineered cell of claim 36, wherein said antibody is an IgG, or a recombinant IgG antibody or antibody fragment comprising an Fc portion mutated to alter (eliminate or enhance) FcR or FcRn interactions, to increase half-life and/or increase therapeutic efficacy, such as a LALA, LALA PG, N297, GASD/ALIE, DHS, YTE or LS mutation or glycan modified to alter (eliminate or enhance) FcR or FcRn interactions such as enzymatic or chemical addition or removal of glycans or expression in a cell line engineered with a defined glycosylating pattern.

46. The hybridoma or engineered cell of claim 36, wherein said antibody or antibody fragment further comprises a cell penetrating peptide and/or is an intrabody.

47. A vaccine formulation comprising one or more antibodies or antibody fragments characterized by clone-paired heavy and light chain CDR sequences from Tables 3 and 4, respectively.

48. The vaccine formulation of claim 47, wherein at least one of said antibodies or antibody fragments is encoded by light and heavy chain variable sequences according to clone-paired sequences from Table 1.

49. The vaccine formulation of claim 47, wherein at least one of said antibodies or antibody fragments is encoded by light and heavy chain variable sequences having at least 70%, 80%, or 90% identity to clone-paired sequences from Table 1.

50. The vaccine formulation of claim 47, wherein at least one of said antibodies or antibody fragments is encoded by light and heavy chain variable sequences having at least 95% identity to clone-paired sequences from Table 1.

51. The vaccine formulation of claim 47, wherein at least one of said antibodies or antibody fragments comprises light and heavy chain variable sequences according to clone-paired sequences from Table 2.

52. The vaccine formulation of claim 47, wherein at least one of said antibodies or antibody fragments comprises light and heavy chain variable sequences having 95% identity to clone-paired sequences from Table 2.

53. The vaccine formulation of claim 47, wherein at least one of said antibody fragments is a recombinant scFv (single chain fragment variable) antibody, Fab fragment, F(ab′)₂ fragment, or Fv fragment.

54. The vaccine formulation of claim 47, wherein at least one of said antibodies is a chimeric antibody or is bispecific antibody.

55. The vaccine formulation of claim 47, wherein said antibody is an IgG, or a recombinant IgG antibody or antibody fragment comprising an Fc portion mutated to alter (eliminate or enhance) FcR or FcRn interactions, to increase half-life and/or increase therapeutic efficacy, such as a LALA, LALA PG, N297, GASD/ALIE, DHS, YTE or LS mutation or glycan modified to alter (eliminate or enhance) FcR or FcRn interactions such as enzymatic or chemical addition or removal of glycans or expression in a cell line engineered with a defined glycosylating pattern.

56. The vaccine formulation of claim 47, wherein at least one of said antibodies or antibody fragments further comprises a cell penetrating peptide and/or is an intrabody.

57. A vaccine formulation comprising one or more expression vectors encoding a first antibody or antibody fragment characterized by clone-paired heavy and light chain CDR sequences from Tables 3 and 4, respectively.

58. The vaccine formulation of claim 57, wherein said expression vector(s) is/are Sindbis virus or VEE vector(s).

59. The vaccine formulation of claim 57, formulated for delivery by needle injection, jet injection, or electroporation.

60. The vaccine formulation of claim 57, further comprising one or more expression vectors encoding for a second antibody or antibody fragment, such as a distinct antibody or antibody fragment characterized by clone-paired heavy and light chain CDR sequences from Tables 3 and 4, respectively.

61. A method of protecting the health of a placenta and/or fetus of a pregnant a subject infected with or at risk of infection with dengue virus comprising delivering to said subject an antibody or antibody fragment having clone-paired heavy and light chain CDR sequences from Tables 3 and 4, respectively.

62. The method of claim 61, the antibody or antibody fragment is encoded by clone-paired light and heavy chain variable sequences as set forth in Table 1.

63. The method of claim 61, the antibody or antibody fragment is encoded by clone-paired light and heavy chain variable sequences having 95% identity to as set forth in Table 1.

64. The method of claim 61, wherein said antibody or antibody fragment is encoded by light and heavy chain variable sequences having 70%, 80%, or 90% identity to clone-paired sequences from Table 1.

65. The method of claim 61, wherein said antibody or antibody fragment comprises light and heavy chain variable sequences according to clone-paired sequences from Table 2.

66. The method of claim 61, wherein said antibody or antibody fragment comprises light and heavy chain variable sequences having 70%, 80% or 90% identity to clone-paired sequences from Table 2.

67. The method of claim 61, wherein said antibody or antibody fragment comprises light and heavy chain variable sequences having 95% identity to clone-paired sequences from Table 2.

68. The method of claim 61, wherein the antibody fragment is a recombinant scFv (single chain fragment variable) antibody, Fab fragment, F(ab′)₂ fragment, or Fv fragment.

69. The method of claim 61, wherein said antibody is an IgG, or a recombinant IgG antibody or antibody fragment comprising an Fc portion mutated to alter (eliminate or enhance) FcR or FcRn interactions, to increase half-life and/or increase therapeutic efficacy, such as a LALA, LALA PG, N297, GASD/ALIE, DHS, YTE or LS mutation or glycan modified to alter (eliminate or enhance) FcR or FcRn interactions such as enzymatic or chemical addition or removal of glycans or expression in a cell line engineered with a defined glycosylating pattern.

70. The method of claim 61, wherein said antibody is a chimeric antibody or a bispecific antibody.

71. The method of claim 61, wherein said antibody or antibody fragment is administered prior to infection or after infection.

72. The method of claim 61, wherein said subject is a pregnant female, a sexually active female, or a female undergoing fertility treatments.

73. The method of claim 61, wherein delivering comprises antibody or antibody fragment administration, or genetic delivery with an RNA or DNA sequence or vector encoding the antibody or antibody fragment.

74. The method of claim 61, wherein the antibody or antibody fragment increases the size of the placenta as compared to an untreated control.

75. The method of claim 61, wherein the antibody or antibody fragment reduces viral load and/or pathology of the fetus as compared to an untreated control.

76. A method of determining the antigenic integrity, correct conformation and/or correct sequence of a Dengue virus antigen comprising:
(a) contacting a sample comprising said antigen with a first antibody or antibody fragment having clone-paired heavy and light chain CDR sequences from Tables 3 and 4, respectively; and
(b) determining antigenic integrity, correct conformation and/or correct sequence of said antigen by detectable binding of said first antibody or antibody fragment to said antigen.

77. The method of claim 76, wherein said sample comprises recombinantly produced antigen.

78. The method of claim 76, wherein said sample comprises a vaccine formulation or vaccine production batch.

79. The method of claim 76, wherein detection comprises ELISA, RIA, western blot, a biosensor using surface plasmon resonance or biolayer interferometry, or flow cytometric staining.

80. The method of claim 76, wherein the first antibody or antibody fragment is encoded by clone-paired variable sequences as set forth in Table 1.

81. The method of claim 76, wherein said first antibody or antibody fragment is encoded by light and heavy chain variable sequences having 70%, 80%, or 90% identity to clone-paired variable sequences as set forth in Table 1.

82. The method of claim 76, wherein said first antibody or antibody fragment is encoded by light and heavy chain variable sequences having 95% identity to clone-paired sequences as set forth in Table 1.

83. The method of claim 76, wherein said first antibody or antibody fragment comprises light and heavy chain variable sequences according to clone-paired sequences from Table 2.

84. The method of claim 76, wherein said first antibody or antibody fragment comprises light and heavy chain variable sequences having 70%, 80% or 90% identity to clone-paired sequences from Table 2.

85. The method of claim 76, wherein said first antibody or antibody fragment comprises light and heavy chain variable sequences having 95% identity to clone-paired sequences from Table 2.

86. The method of claim 76, wherein the first antibody fragment is a recombinant scFv (single chain fragment variable) antibody, Fab fragment, F(ab′)₂ fragment, or Fv fragment.

87. The method of claim 76, further comprising performing steps (a) and (b) a second time to determine the antigenic stability of the antigen over time.

88. The method of claim 76, further comprising:
(c) contacting a sample comprising said antigen with a second antibody or antibody fragment having clone-paired heavy and light chain CDR sequences from Tables 3 and 4, respectively; and
(d) determining antigenic integrity of said antigen by detectable binding of said second antibody or antibody fragment to said antigen.

89. The method of claim 88, wherein the second antibody or antibody fragment is encoded by clone-paired variable sequences as set forth in Table 1.

90. The method of claim 89, wherein said second antibody or antibody fragment is encoded by light and heavy chain variable sequences having 70%, 80%, or 90% identity to clone-paired variable sequences as set forth in Table 1.

91. The method of claim 89, wherein said second antibody or antibody fragment is encoded by light and heavy chain variable sequences having 95% identity to clone-paired sequences as set forth in Table 1.

92. The method of claims 89, wherein said second antibody or antibody fragment comprises light and heavy chain variable sequences according to clone-paired sequences from Table 2.

93. The method of claim 89, wherein said second antibody or antibody fragment comprises light and heavy chain variable sequences having 70%, 80% or 90% identity to clone-paired sequences from Table 2.

94. The method of claim 89, wherein said second antibody or antibody fragment comprises light and heavy chain variable sequences having 95% identity to clone-paired sequences from Table 2.

95. The method of claim 89, wherein the second antibody fragment is a recombinant scFv (single chain fragment variable) antibody, Fab fragment, F(ab′)₂ fragment, or Fv fragment.

96. The method of claim 89, further comprising performing steps (c) and (d) a second time to determine the antigenic stability of the antigen over time.

97. A human monoclonal antibody or antibody fragment, or hybridoma or engineered cell producing the same, wherein said antibody binds to Dengue virus serotype 3 and does not bind to other Dengue virus serotypes.

98. A human monoclonal antibody or antibody fragment, or hybridoma or engineered cell producing the same, wherein said antibody binds in a serotype 3-specific manner to an epitope in dengue virus type 3 E glycoprotein domain I, or an epitope in domain II, or a quaternary epitope comprising residues in domains I and II.
